SEQUENCE LISTING (134 sequences)

SEQ ID NO: 1 - protein, 11 aa, synthetic construct
KIGDFGLATVK

SEQ ID NO: 2 - protein, 11 aa, synthetic construct
KIGDFGLATEK

SEQ ID NO: 3 - protein, 10 aa, synthetic construct
KLVVVGAGGV

SEQ ID NO: 4 - protein, 10 aa, synthetic construct
KLVVVGADGV

SEQ ID NO: 5 - skipped

SEQ ID NO: 6 - protein, 10 aa, synthetic construct
KLVVVGAVGV

SEQ ID NO: 7 - protein, 8 aa, synthetic construct
KLVVVGAG

SEQ ID NO: 8 - protein, 8 aa, synthetic construct
KLVVVGAV

SEQ ID NO: 9 - protein, 11 aa, synthetic construct
KIGDFGLATVK

SEQ ID NO: 10 - protein, 11 aa, synthetic construct
KIGDFGLATEK

SEQ ID NO: 11 - protein, 10 aa, synthetic construct
AHHGGWTTKM

SEQ ID NO: 12 - protein, 10 aa, synthetic construct
ARHGGWTTKM

SEQ ID NO: 13-14 - skipped (2 entries)

SEQ ID NO: 15 - protein, 11 aa, synthetic construct
STRDPLSEITE

SEQ ID NO: 16 - protein, 11 aa, synthetic construct
STRDPLSEITK

SEQ ID NO: 17 - protein, 10 aa, synthetic construct
FGLATVKSRW

SEQ ID NO: 18 - protein, 10 aa, synthetic construct
FGLATEKSRW

SEQ ID NO: 19 - protein, 8 aa, synthetic construct
LATVKSRW

SEQ ID NO: 20 - protein, 8 aa, synthetic construct
LATEKSRW

SEQ ID NO: 21 - DNA, 28 bp, synthetic construct
  source 1..28: mol_type = other DNA
ctcgcgctactctctctttctggcctgg

SEQ ID NO: 22 - DNA, 129 bp, synthetic construct
  source 1..129: mol_type = other DNA
gccttagctgtgctcgcgctactctctctttctggcctggaggctatccagcgtgagtctctcctaccctcccgctctggtccttcctctccgctctgcaccctctgtggccctcgctgtgctctctcg

SEQ ID NO: 23 - DNA, 130 bp, synthetic construct
  source 1..130: mol_type = other DNA
cggaatcgacacgagcgcgatgagagagaaagaccggacctcccgataggtcgcactcagagaggatgggagggcgagaccaggaaggagaggcgagacgtgggagacaccgggagcgacacgagagagc

SEQ ID NO: 24 - DNA, 28 bp, synthetic construct
  source 1..28: mol_type = other DNA
gagcgcgatgagagagaaagaccggacc

SEQ ID NO: 25 - protein, 17 aa, synthetic construct
ALAVLALLSLSGLEAIQ

SEQ ID NO: 26 - DNA, 23 bp, synthetic construct
  source 1..23: mol_type = other DNA
gctactctctctttctggccngg

SEQ ID NO: 27 - DNA, 23 bp, synthetic construct
  source 1..23: mol_type = other DNA
actctctctttctggcctggngg

SEQ ID NO: 28 - DNA, 24 bp, synthetic construct
  source 1..24: mol_type = other DNA
ctactctctctttctggcctngag

SEQ ID NO: 29 - DNA, 24 bp, synthetic construct
  source 1..24: mol_type = other DNA
gccagaaagagagagtagcgngag

SEQ ID NO: 30 - DNA, 23 bp, synthetic construct
  source 1..23: mol_type = other DNA
ggacatctggctaaggaactngg

SEQ ID NO: 31 - DNA, 23 bp, synthetic construct
  source 1..23: mol_type = other DNA
ccagaggagcttccggcagacct

SEQ ID NO: 32 - DNA, 131 bp, synthetic construct
  source 1..131: mol_type = other DNA
ctgcagccttcccagaggagcttccggcagacctgaagcactggaagccaggtgtgcagggcaggtgggctggggttgggaagggtggatgccttggggaggggatggaagagatttgaactcctggccca

SEQ ID NO: 33 - DNA, 130 bp, synthetic construct
  source 1..130: mol_type = other DNA
gacgtcggaagggtctcctcgaaggccgtctggacttcgtgaccttcggtccacacgtcccgtccacccgaccccaacccttcccacctacggaaccctcccctaccttctctaaacttgaggaccgggt

SEQ ID NO: 34 - DNA, 23 bp, synthetic construct
  source 1..23: mol_type = other DNA
ggtctcctcgaaggccgtctgga

SEQ ID NO: 35 - protein, 15 aa, synthetic construct
PFPEELPADLKHYKP

SEQ ID NO: 36 - DNA, 23 bp, synthetic construct
  source 1..23: mol_type = other DNA
ccagaggagcttccggcagacct

SEQ ID NO: 37 - DNA, 23 bp, synthetic construct
  source 1..23: mol_type = other DNA
ggtctgccggaagctcctctngg

SEQ ID NO: 38 - DNA, 23 bp, synthetic construct
  source 1..23: mol_type = other DNA
gacctgaagcactggaagccngg

SEQ ID NO: 39 - DNA, 24 bp, synthetic construct
  source 1..24: mol_type = other DNA
ttcccagaggagcttccggcngac

SEQ ID NO: 40 - DNA, 24 bp, synthetic construct
  source 1..24: mol_type = other DNA
agaggagcttccggcagaccngaa

SEQ ID NO: 41 - DNA, 24 bp, synthetic construct
  source 1..24: mol_type = other DNA
tccggcagacctgaagcactngaa

SEQ ID NO: 42 - DNA, 23 bp, synthetic construct
  source 1..23: mol_type = other DNA
tgccggaagctcctctgggangg

SEQ ID NO: 43 - DNA, 28 bp, synthetic construct
  source 1..28: mol_type = other DNA
cgagatgtctcgctccgtggccttagct

SEQ ID NO: 44 - DNA, 28 bp, synthetic construct
  source 1..28: mol_type = other DNA
gctctacagagcgaggcaccggaatcga

SEQ ID NO: 45 - DNA, 26 bp, synthetic construct
  source 1..26: mol_type = other DNA
cgagatgtctcgctccgtggccttag

SEQ ID NO: 46 - DNA, 30 bp, synthetic construct
  source 1..30: mol_type = other DNA
cgagatgtctcgctccgtggccttagctct

SEQ ID NO: 47 - DNA, 23 bp, synthetic construct
  source 1..23: mol_type = other DNA
cgagatgtctcgctccgtggcct

SEQ ID NO: 48 - DNA, 48 bp, synthetic construct
  source 1..48: mol_type = other DNA
gtgctcgcgctactctctctttctggcctggaggctatccagcgtgag

SEQ ID NO: 49 - DNA, 48 bp, synthetic construct
  source 1..48: mol_type = other DNA
cacgagcgcgatgagagagaaagaccggacctccgataggtcgcactc

SEQ ID NO: 50 - DNA, 92 bp, synthetic construct
  source 1..92: mol_type = other DNA
cacatgttttctctgcagccttcccagaggagcttccggcagacctgaagcactggaagccaggtgtgcagggcaggtgggctggggttggg

SEQ ID NO: 51 - DNA, 93 bp, synthetic construct
  source 1..93: mol_type = other DNA
gtgtacaaaagagacgtcggaagggtctcctcgaaggccgtctggacttcgtgaccttcggtccacacgtcccgtcccacccgaccccaaccc

SEQ ID NO: 52 - DNA, 82 bp, synthetic construct
  source 1..82: mol_type = other DNA
cacatgttttctctgcagccttcccagagcagacctgaagcactggaagccaggtgtgcagggcaggtgggctggggttggg

SEQ ID NO: 53 - DNA, 81 bp, synthetic construct
  source 1..81: mol_type = other DNA
cacatgttttctctgcagcagcttccggcagacctgaagcactggaagccaggtgtgcagggcaggtgggctggggttggg

SEQ ID NO: 54 - DNA, 91 bp, synthetic construct
  source 1..91: mol_type = other DNA
cacatgttttctctgcagccttcccagagagcttccggcagacctgaagcactggaagccaggtgtgcagggcaggtgggctggggttggg

SEQ ID NO: 55 - DNA, 79 bp, synthetic construct
  source 1..79: mol_type = other DNA
cacatgttttctctgcagccttccggcagacctgaagcactggaagccaggtgtgcagggcaggtgggctggggttggg

SEQ ID NO: 56 - DNA, 259 bp, synthetic construct
  source 1..259: mol_type = other DNA
tgggttggggcaaagagggaaatgagatcatgtccaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaac

SEQ ID NO: 57 - DNA, 260 bp, synthetic construct
  source 1..260: mol_type = other DNA
acccaaccccgtttctccctttactctagtacaggattgggactaggagaacagggtgtctataggtcttgggactgggacggcacatggtcgactctctgagatttaggtcactgttcagacagacggataagtggctaaaactaagagtttgtttacacagtgtttcattcctaagactacacatatagtgtctgttttgacacgatctgtactccagatacctgaagttctcgttgtcacgacaccggacctcgttg

SEQ ID NO: 58-59 - skipped (2 entries)

SEQ ID NO: 60 - DNA, 23 bp, synthetic construct
  source 1..23: mol_type = other DNA
tcagggttctggatatctgtngg

SEQ ID NO: 61 - DNA, 23 bp, synthetic construct
  source 1..23: mol_type = other DNA
gagaatcaaaatcggtgaatngg

SEQ ID NO: 62 - DNA, 23 bp, synthetic construct
  source 1..23: mol_type = other DNA
tggatttagagtctctcagcngg

SEQ ID NO: 63 - DNA, 23 bp, synthetic construct
  source 1..23: mol_type = other DNA
tctctcagctggtacacggcngg

SEQ ID NO: 64 - DNA, 23 bp, synthetic construct
  source 1..23: mol_type = other DNA
acacggcagggtcagggttcngg

SEQ ID NO: 65 - DNA, 23 bp, synthetic construct
  source 1..23: mol_type = other DNA
gctggtacacggcagggtcangg

SEQ ID NO: 66 - DNA, 23 bp, synthetic construct
  source 1..23: mol_type = other DNA
atctgtgggacaagaggatcngg

SEQ ID NO: 67 - DNA, 23 bp, synthetic construct
  source 1..23: mol_type = other DNA
gtcagggttctggatatctgngg

SEQ ID NO: 68 - DNA, 23 bp, synthetic construct
  source 1..23: mol_type = other DNA
ctctcagctggtacacggcangg

SEQ ID NO: 69 - DNA, 23 bp, synthetic construct
  source 1..23: mol_type = other DNA
ctggatatctgtgggacaagngg

SEQ ID NO: 70 - DNA, 23 bp, synthetic construct
  source 1..23: mol_type = other DNA
agctggtacacggcagggtcngg

SEQ ID NO: 71 - DNA, 23 bp, synthetic construct
  source 1..23: mol_type = other DNA
tctgtgggacaagaggatcangg

SEQ ID NO: 72 - DNA, 23 bp, synthetic construct
  source 1..23: mol_type = other DNA
agagtctctcagctggtacangg

SEQ ID NO: 73 - DNA, 23 bp, synthetic construct
  source 1..23: mol_type = other DNA
atttgtttgagaatcaaaatngg

SEQ ID NO: 74 - DNA, 23 bp, synthetic construct
  source 1..23: mol_type = other DNA
taggcagacagacttgtcacngg

SEQ ID NO: 75 - DNA, 23 bp, synthetic construct
  source 1..23: mol_type = other DNA
gggacaagaggatcagggttngg

SEQ ID NO: 76 - DNA, 130 bp, synthetic construct
  source 1..130: mol_type = other DNA
cctaccagaaccagacagctctcagagcaaccctggctccaacccctcttccctttccagaggacctgaacaaggtgttcccacccgaggtcgctgtgtttgagccatcagaagcagagatctcccacac

SEQ ID NO: 77 - DNA, 130 bp, synthetic construct
  source 1..130: mol_type = other DNA
ggatggtcttggtctgtcgagagtctcgttgggaccgaggttggggagaagggaaaggtctcctggacttgttccacaagggtgggctccagcgacacaaactcggtagtcttcgtctctagagggtgtg

SEQ ID NO: 78 - protein, 23 aa, synthetic construct
DLNKVFPPEVAVFEPSEAEISHT

SEQ ID NO: 79 - DNA, 23 bp, synthetic construct
  source 1..23: mol_type = other DNA
gaacaaggtgttcccacccgngg

SEQ ID NO: 80 - protein, 23 aa, synthetic construct
CTTTCCAGAGGACCTGAACANGG

SEQ ID NO: 81 - DNA, 23 bp, synthetic construct
  source 1..23: mol_type = other DNA
cgggtgggaacaccttgttcngg

SEQ ID NO: 82 - DNA, 23 bp, synthetic construct
  source 1..23: mol_type = other DNA
ggtcctctggaaagggaagangg

SEQ ID NO: 83 - DNA, 23 bp, synthetic construct
  source 1..23: mol_type = other DNA
aacaccttgttcaggtcctcngg

SEQ ID NO: 84 - DNA, 23 bp, synthetic construct
  source 1..23: mol_type = other DNA
cttgttcaggtcctctggaangg

SEQ ID NO: 85 - DNA, 23 bp, synthetic construct
  source 1..23: mol_type = other DNA
gtcctctggaaagggaagagngg

SEQ ID NO: 86 - DNA, 23 bp, synthetic construct
  source 1..23: mol_type = other DNA
caaacacagcgacctcgggtngg

SEQ ID NO: 87 - DNA, 23 bp, synthetic construct
  source 1..23: mol_type = other DNA
tcaaacacagcgacctcgggngg

SEQ ID NO: 88 - DNA, 23 bp, synthetic construct
  source 1..23: mol_type = other DNA
ttgttcaggtcctctggaaangg

SEQ ID NO: 89 - DNA, 23 bp, synthetic construct
  source 1..23: mol_type = other DNA
ggctcaaacacagcgacctcngg

SEQ ID NO: 90 - DNA, 23 bp, synthetic construct
  source 1..23: mol_type = other DNA
aaaggccacactggtgtgccngg

SEQ ID NO: 91 - DNA, 23 bp, synthetic construct
  source 1..23: mol_type = other DNA
acactggtgtgcctggccacngg

SEQ ID NO: 92 - DNA, 23 bp, synthetic construct
  source 1..23: mol_type = other DNA
agagatctcccacacccaaangg

SEQ ID NO: 93 - DNA, 23 bp, synthetic construct
  source 1..23: mol_type = other DNA
aggtcctctggaaagggaagngg

SEQ ID NO: 94 - DNA, 23 bp, synthetic construct
  source 1..23: mol_type = other DNA
tggctcaaacacagcgacctngg

SEQ ID NO: 95 - DNA, 23 bp, synthetic construct
  source 1..23: mol_type = other DNA
ggcacaccagtgtggcctttngg

SEQ ID NO: 96 - DNA, 23 bp, synthetic construct
  source 1..23: mol_type = other DNA
tgggagatctctgcttctgangg

SEQ ID NO: 97 - DNA, 23 bp, synthetic construct
  source 1..23: mol_type = other DNA
cagtgtggccttttgggtgtngg

SEQ ID NO: 98 - DNA, 23 bp, synthetic construct
  source 1..23: mol_type = other DNA
tgtggccaggcacaccagtgngg

SEQ ID NO: 99 - DNA, 23 bp, synthetic construct
  source 1..23: mol_type = other DNA
ccagtgtggccttttgggtgngg

SEQ ID NO: 100 - DNA, 23 bp, synthetic construct
  source 1..23: mol_type = other DNA
ccacacccaaaaggccacacngg

SEQ ID NO: 101 - DNA, 23 bp, synthetic construct
  source 1..23: mol_type = other DNA
gcacaccagtgtggccttttngg

SEQ ID NO: 102 - DNA, 35 bp, synthetic construct
  source 1..35: mol_type = other DNA
gactctaaatccagtgacaagtctgtctgcctatt

SEQ ID NO: 103 - DNA, 35 bp, synthetic construct
  source 1..35: mol_type = other DNA
ctgagatttaggtcactgttcagacagacggataa

SEQ ID NO: 104 - DNA, 22 bp, synthetic construct
  source 1..22: mol_type = other DNA
taaatccagtgacaagtctgtc

SEQ ID NO: 105 - DNA, 52 bp, synthetic construct
  source 1..52: mol_type = other DNA
caccgattttgattctcaaacaaatgtgtcacaaagtaaggattctgatgtg

SEQ ID NO: 106 - DNA, 52 bp, synthetic construct
  source 1..52: mol_type = other DNA
gtggctaaaactaagagtttgtttacacagtgtttcattcctaagactacac

SEQ ID NO: 107 - DNA, 51 bp, synthetic construct
  source 1..51: mol_type = other DNA
accgattttgattctcaaacaaatgtgtcacaaagtaaggattctgatgtg

SEQ ID NO: 108 - DNA, 54 bp, synthetic construct
  source 1..54: mol_type = other DNA
ttcaccgattttgattctcaaacaaatgtgtcacaaagtaaggattctgatgtg

SEQ ID NO: 109 - DNA, 95 bp, synthetic construct
  source 1..95: mol_type = other DNA
ttccctttccagaggacctgaacaaggtgttcccacccgaggtcgctgtgtttgagccatcagaagcagagatctcccacacccaaaaggccaca

SEQ ID NO: 110 - DNA, 94 bp, synthetic construct
  source 1..94: mol_type = other DNA
aagggaaaggtctcctggacttgttccacaagggtgggctccagcgacacaaactcggtagtcttcgtctctagaggtgtgggttttccggtgt

SEQ ID NO: 111 - protein, 27 aa, synthetic construct
KSSDKSVCLFTDFDSQTNVSQSKDSDV

SEQ ID NO: 112 - protein, 27 aa, synthetic construct
DLNKVFPPEVAVFEPSEAEISHTQKAT

SEQ ID NO: 113 - DNA, 75 bp, synthetic construct
  source 1..75: mol_type = other DNA
ttccctttccagaggcccgaggtcgctgtgtttgagccatcagaagcagagatctcccacacccaaaaggccaca

SEQ ID NO: 114 - DNA, 94 bp, synthetic construct
  source 1..94: mol_type = other DNA
ttccctttccagaggacctgaacaaggtgttccgaccgaggtcgctgtgtttgagccatcagaagcagagatctcccacacccaaaaggccaca

SEQ ID NO: 115 - skipped

SEQ ID NO: 116 - DNA, 93 bp, synthetic construct
  source 1..93: mol_type = other DNA
ttccctttccagaggacctgaacaaggtgttcccccgaggtcgctgtgtttgagccatcagaagcagagatctcccacacccaaaaggccaca

SEQ ID NO: 117 - DNA, 130 bp, synthetic construct
  source 1..130: mol_type = other DNA
ctatctcctggcgataaccaattttcccaccatctttcctcttaggagacttgcctggtgaaaatcatcactggtcttttggagtttgaggtatacctagagtacctccagaacagatttgagagtagtg

SEQ ID NO: 118 - DNA, 74 bp, synthetic construct
  source 1..74: mol_type = other DNA
gatagaggaccgctattggttaaaagggtggtagaaaggagaatcctctgaacggaccacttttagtagtgacc

SEQ ID NO: 119 - DNA, 23 bp, synthetic construct
  source 1..23: mol_type = other DNA
cctggtgaaaatcatcactggtc

SEQ ID NO: 120 - DNA, 23 bp, synthetic construct
  source 1..23: mol_type = other DNA
ggaccacttttagtagtgaccag

SEQ ID NO: 121 - protein, 27 aa, synthetic construct
ETCLVKIITGLLFEVYLEYLQNRFESS

SEQ ID NO: 122 - DNA, 23 bp, synthetic construct
  source 1..23: mol_type = other DNA
tgcctggtgaaaatcatcacngg

SEQ ID NO: 123 - DNA, 23 bp, synthetic construct
  source 1..23: mol_type = other DNA
gaccagtgatgattttcaccngg

SEQ ID NO: 124 - DNA, 23 bp, synthetic construct
  source 1..23: mol_type = other DNA
gaaaatcatcactggtctttngg

SEQ ID NO: 125 - DNA, 23 bp, synthetic construct
  source 1..23: mol_type = other DNA
ttcctcttaggagacttgccngg

SEQ ID NO: 126 - DNA, 25 bp, synthetic construct
  source 1..25: mol_type = other DNA
ctcttaggagacttgcctggngaaa

SEQ ID NO: 127 - DNA, 24 bp, synthetic construct
  source 1..24: mol_type = other DNA
ttcaccaggcaagtctcctangag

SEQ ID NO: 128 - DNA, 24 bp, synthetic construct
  source 1..24: mol_type = other DNA
accaggcaagtctcctaagangaa

SEQ ID NO: 129 - DNA, 23 bp, synthetic construct
  source 1..23: mol_type = other DNA
ggcaagtctcctaagaggaanga

SEQ ID NO: 130 - DNA, 23 bp, synthetic construct
  source 1..23: mol_type = other DNA
caccaggcaagtctcctaagngg

SEQ ID NO: 131 - protein, 22 aa, synthetic construct
MSRSVALAVLALLSLSGLEAIQ

SEQ ID NO: 132 - protein, 66 aa, synthetic construct
IQNPDPAVYQLRDSKSSDKSVCLFTDFDSQTNVSQSKDSDVYITDKTVLDMRSMDFKSNSAVAWSN

SEQ ID NO: 133 - DNA, 23 bp, synthetic construct
  source 1..23: mol_type = other DNA
gagtagcgcgagcacagctangg

SEQ ID NO: 134 - DNA, 23 bp, synthetic construct
  source 1..23: mol_type = other DNA
ctcgcgctactctctctttcngg

(19) United States
(12) Patent Application Publication
(10) Pub. No.: US 2026/0345679 A1
(43) Pub. Date: Nov. 5, 2026

(54) HYPOIMMUNOGENIC CELLS FOR GENERATING BIOMIMETIC NANOVESICLES

(71) Applicant: Thomas MALCOLM, Andover, NJ (US)

(72) Inventor: Thomas MALCOLM, Andover, NJ (US)

(21) Appl. No.: 18/868,356

(22) PCT Filed: May 25, 2023
(86) PCT No.: PCT/US2023/067488

§ 371 Date: Nov. 22, 2024

Related U.S. Application Data
(60) Provisional application No. 63/345,669, filed on May 25, 2022.
(60) Provisional application No. 63/387,516, filed on Dec. 15, 2022.

(51) Int. Cl. A61K 35/17; A61K 35/15; A61K 40/11; A61K 40/15; A61K 40/17; A61K 40/31; A61K 40/42; C07K 14/52; C07K 14/705; C07K 16/28; C12N 5/0783; C12N 5/0786; C12N 15/113; C12N 15/86; C12N 15/90

(52) U.S. Cl. CPC A61K 35/17; A61K 35/15; A61K 40/11; A61K 40/15; A61K 40/17; A61K 40/31; A61K 40/4217; C07K 14/523; C07K 14/7051; C07K 14/70514; C07K 14/70517; C07K 14/70521; C07K 14/70532; C07K 14/70575; C07K 14/70578; C07K 14/70596; C07K 16/2866; C12N 5/0636; C12N 5/0645; C12N 5/0646; C12N 15/113; C12N 15/86; C12N 15/907; A61K 2239/13; A61K 2239/21; A61K 2239/22; A61K 2239/29; C07K 2317/622; C12N 2310/14; C12N 2506/45; C12N 2510/00

(57) ABSTRACT

Disclosed herein are methods of generating hypoimmunogenic cells and methods of using hypoimmunogenic cells for treating mammalian diseases and for preparing biomimetic nanovesicles (BioNVs).

### CROSS-REFERENCE TO RELATED APPLICATIONS

This application claims the benefit of and priority to U.S. Provisional Application No. 63/345,669, filed May 25, 2022, and U.S. Provisional Application No. 63/387,516, filed Nov. 14, 2022, the contents of which are herein incorporated by reference in their entirety.

### TECHNICAL FIELD

The present disclosure provides, in part, methods of generating hypoimmunogenic cells for generating biomimetic nanovesicles (BioNVs) and compositions and uses of the same.

### DESCRIPTION OF THE TEXT FILE SUBMITTED ELECTRONICALLY

The contents of the text file submitted electronically herewith are incorporated herein by reference in their entirety: a computer-readable XML format copy of the Sequence Listing (filename: “CAR-006PC_Sequence_Listing.xml”; date recorded May 24, 2023; file size: 117,233 bytes).

### BACKGROUND

Whole cell therapies are becoming an increasingly efficacious class of therapeutics. Cell therapies are administered by transfusion of whole cells from donors into a patient and requires rigorous immunogenicity matching between the donor and recipient. Multiple hypoimmunogenic engineering techniques of induced pluripotent stem cells (iPSCs) have been reported. Each approach aims to eliminate the human leukocyte antigen (HLA) genes that encode the multiple histocompatibility complex (MHC) membrane glycoproteins that confer immune reactions associated Graft Versus Host Disease (GVHD) rejections. The HLA gene cluster can be divided into three categories: 1) the MHC class I pathway, 2) the MHC class II pathway, and 3) the MHC class III pathway. Only the MHC class I and II pathways express the protein complexes that elicit an immune response in GVHD, whereas MHC class III complexes are not involved in immunization activities.

Although the elimination, or suppression, of these complexes affords some protection against deleterious immune responses in the patient, there are additional surface proteins and mechanisms that are required to be eliminated/suppressed to enhance cellular hypoimmune properties. For example, the elimination of the MHC class I/II protein complexes triggers NK cells and macrophages into an “active clearance mode,” where the cells are subsequently destroyed.

While some systems are promising, they still express minor non-MHC antigens, can have genetic polymorphisms that contribute to immunological rejection, have significant teratoma potential, and can lead to the eventual development of humoral immunity against the cells. In addition, even with hypoimmunogenic engineering, whole cell therapeutic approaches have the potential for cytokine release syndrome (CRS), cell exhaustion, tissue and/or tumor penetration issues, biological barrier crossing issues, payload packaging difficulties, and targeting issues (e.g., off-targeting effects), to name a few. Additionally, hypoimmunogenic cells can require additional engineering to avoid persistence and to initiate controlled cell death (e.g., “suicide switches”). With each round of manipulation, manufacturing costs rise with whole cell therapeutics. There remains a need to combine the hypoimmunogenic qualities and the disease-specific targeting of engineered whole cell therapies while circumventing the issues and manufacturing costs to enable viable therapeutic options to the clinic.

### SUMMARY

In embodiments, described herein are methods of generating a hypoimmunogenic cell comprising reducing or ablating the expression and/or activity of one or more immunogenic proteins in a cell, and expressing or increasing expression and/or activity of one or more immunoprotective proteins in the cell, thereby generating the hypoimmunogenic cell. In embodiments, described herein are hypoimmunogenic cells produced using the present methods.

In embodiments, the cell is a stem cell, an induced pluripotent stem cell (iPSC), a reprogrammed pluripotent or multipotent cell, an embryonic stem cell, a mesenchymal stem cell, or a differentiated cell which originates from any stem cell thereof. In embodiments, the differentiated cell is a T cell, helper T cell, T-memory cell, or NK cell. In embodiments, the differentiated cell is a macrophage. In embodiments, the differentiated cell is a monocyte. In embodiments, the differentiated cell is a hepatocyte, cardiomyocyte, neuron, endothelial cell, pancreatic cell, or retinal pigmented epithelium (RPE) cell.

In embodiments, the hypoimmunogenic cell substantially lacks one or more of MHC class I protein complexes, MHC class II complexes, T cell receptor (TCR) complexes, and/or cytokine release syndrome (CRS) proteins. In embodiments, reducing or ablating the expression and/or activity of one or more immunogenic proteins comprises a B2-macroglobulin (B2M) gene disruption and/or a disruption that reduces or ablates MHC class I protein expression and/or activity. In embodiments, reducing or ablating the expression and/or activity of one or more immunogenic proteins comprises a CIITA gene disruption and/or a disruption that reduces or ablates MHC class II protein expression and/or activity.

In embodiments, reducing or ablating the expression and/or activity of one or more immunogenic proteins comprises an HLA-A gene disruption and/or a disruption that reduces or ablates HLA-A protein expression and/or activity. In embodiments, reducing or ablating the expression and/or activity of one or more immunogenic proteins comprises an HLA-B gene disruption and/or a disruption that reduces or ablates HLA-B protein expression and/or activity. In embodiments, reducing or ablating the expression and/or activity of one or more immunogenic proteins comprises an HLA-C gene disruption and/or a disruption that reduces or ablates HLA-C protein expression and/or activity. In embodiments, reducing or ablating the expression and/or activity of one or more immunogenic proteins comprises an HLA-E gene disruption or an HLA-G gene disruption and/or a disruption that reduces or ablates HLA-E or HLA-G protein expression and/or activity. In embodiments, reducing or ablating the expression and/or activity of one or more immunogenic proteins comprises an HLA-F gene disruption and/or a disruption that reduces or ablates HLA-F protein expression and/or activity.

In embodiments, reducing or ablating the expression and/or activity of one or more immunogenic proteins comprises a T cell alpha constant (TRAC) gene disruption and/or a disruption that reduces or ablates TRAC protein expression and/or activity. In embodiments, reducing or ablating the expression and/or activity of one or more immunogenic proteins comprises a T cell beta constant (TRBC) gene disruption and/or a disruption that reduces or ablates TRBC protein expression and/or activity.

In embodiments, reducing or ablating the expression and/or activity of one or more immunogenic proteins comprises a PD-1 gene disruption and/or a disruption that reduces or ablates PD-1 protein expression and/or activity. In embodiments, reducing or ablating the expression and/or activity of one or more immunogenic proteins comprises an IL-4 gene disruption and/or a disruption that reduces or ablates IL-4 protein expression and/or activity. In embodiments, wherein reducing or ablating the expression and/or activity of one or more immunogenic proteins comprises an IL-6 gene disruption and/or a disruption that reduces or ablates IL-6 protein expression and/or activity. In embodiments, reducing or ablating the expression and/or activity of one or more immunogenic proteins comprises an IL-10 gene disruption and/or a disruption that reduces or ablates IL-10 protein expression and/or activity. In embodiments, reducing or ablating the expression and/or activity of one or more immunogenic proteins comprises an IL-16 gene disruption and/or a disruption that reduces or ablates IL-16 protein expression and/or activity. In embodiments, reducing or ablating the expression and/or activity of one or more immunogenic proteins comprises a SerpinB9 gene disruption and/or a disruption that reduces or ablates SerpinB9 protein expression activity.

In embodiments, reducing or ablating the expression and/or activity of one or more immunogenic proteins is at the DNA level by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, small RNA, or a combination thereof. In embodiments, the small RNA is or comprises one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA. In embodiments, reducing or ablating the expression and/or activity of one or more immunogenic proteins is at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

In embodiments, expressing or increasing expression and/or activity of one or more immunoprotective proteins comprises expressing or increasing expression of a CD34 gene and/or gene product. In embodiments, expressing or increasing expression and/or activity of one or more immunoprotective proteins comprises expressing or increasing expression of a CCL2 gene and/or gene product. In embodiments, expressing or increasing expression and/or activity of one or more immunoprotective proteins comprises expressing or increasing expression of a PD-L1 gene and/or gene product, wherein the cell is not activated. In embodiments, expressing or increasing expression and/or activity of one or more immunoprotective proteins comprises expressing or increasing expression of a H2-M3 gene and/or gene product.

In embodiments, expressing or increasing expression and/or activity of one or more immunoprotective proteins comprises expressing or increasing expression of a CD47 gene and/or gene product. In embodiments, expressing or increasing expression and/or activity of one or more immunoprotective proteins comprises expressing or increasing expression of a CD24 gene and/or gene product. In embodiments, expressing or increasing expression and/or activity of one or more immunoprotective proteins comprises expressing or increasing expression of a chimeric CD24/CD47 gene and/or gene product.

In embodiments, expressing or increasing expression and/or activity of one or more immunoprotective proteins comprises expressing or increasing expression of a CTLA-4 gene and/or gene product.

In embodiments, expressing or increasing expression and/or activity of one or more immunoprotective proteins comprises expressing or increasing expression of a CD200 gene and/or gene product. In embodiments, expressing or increasing expression and/or activity of one or more immunoprotective proteins comprises expressing or increasing expression of a chimeric CD24/CD200 gene and/or gene product or a chimeric CD47/CD200 gene and/or gene product.

In embodiments, expressing or increasing expression and/or activity of one or more immunoprotective proteins comprises expressing or increasing expression of an MFG-E8 gene and/or gene product. In embodiments, expressing or increasing expression and/or activity of one or more immunoprotective proteins comprises expressing or increasing expression of a NCAM gene and/or gene product. In embodiments, expressing or increasing expression and/or activity of one or more immunoprotective proteins comprises expressing or increasing expression of an α-phagocytic integrin gene and/or gene product. In embodiments, expressing or increasing expression and/or activity of one or more immunoprotective proteins comprises expressing or increasing expression of an antibody or antibody format molecule which targets an IL-6 surface receptor (anti-IL-6R).

In embodiments, expressing or increasing expression and/or activity of one or more immunoprotective proteins comprises expression of a FasL gene and/or gene product. In embodiments, expressing or increasing expression and/or activity of one or more immunoprotective proteins does not comprise overexpression of a FasL gene and/or gene product.

In embodiments, the hypoimmunogenic cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins. In embodiments, the hypoimmunogenic cell has expression or has increased expression and/or activity of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the hypoimmunogenic cell has reduced or ablated of a gene and/or gene product of HLA-A, HLA-B, HLA-C, HLA-F, CIITA, IL-6, TRAC, TRBC, and one of either HLA-E or HLA-G. In embodiments, the hypoimmunogenic cell has reduced or ablated of a gene and/or gene product of HLA-A, HLA-B, HLA-C, HLA-F, CIITA, IL-6, TRAC, TRBC, SerpinB9, and one of either HLA-E or HLA-G. In embodiments, the hypoimmunogenic cell has reduced or ablated expression and/or activity of a gene and/or gene product of HLA-A, HLA-B, HLA-C, HLA-F, CIITA, IL-6, TRAC, TRBC, SerpinB9, one of either HLA-E or HLA-G, and one or more of IL-4, IL-10, and IL-16.

In embodiments, the hypoimmunogenic cell expresses or has increased expression of α-phagocytic integrin, CCL2, H2-M3, FasL, MFG-E8, and PD-L1 and/or CTLA-4, wherein the hypoimmunogenic cell does not overexpress FasL, and wherein the hypoimmunogenic cell is not activated with the expression of PD-L1, and one of either CD24, CD47, CD200, chimeric CD24/CD47, chimeric CD24/CD200, and chimeric CD47/CD200, or two of either CD24, CD47, and CD200. In embodiments, the hypoimmunogenic cell expresses or has increased expression of α-phagocytic integrin, CCL2, H2-M3, FasL, MFG-E8, SerpinB9, and PD-L1 and/or CTLA-4, wherein the hypoimmunogenic cell does not overexpress FasL, and wherein the hypoimmunogenic cell is not activated with the expression of PD-L1, and one of either CD24, CD47, CD200, chimeric CD24/CD47, chimeric CD24/CD200, and chimeric CD47/CD200, or two of either CD24, CD47, and CD200.

In embodiments, the hypoimmunogenic cell expresses or has increased expression of a CD200 gene and/or gene product and does not express and/or substantially lacks either a CD24 or a CD47 gene and/or gene product. In embodiments, the hypoimmunogenic cell expresses has no expression and/or activity of a SerpinB9 gene and/or gene product and a CD200 gene and/or gene product.

In embodiments, expressing or increasing expression of the one or more immunoprotective proteins is by introduction of an exogenous genetic element. In embodiments, the introduction of the exogenous genetic element is by stable integration into the cell genome. In embodiments, the stable integration is by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, or a combination thereof. In embodiments, the stable integration is by a viral vector. In embodiments, the introduction of the exogenous genetic element is by transient transfection. In embodiments, expressing or increasing expression of the one or more immunoprotective proteins is by an exogenous promoter and/or enhancer, and/or an endogenous promoter and/or enhancer, or a combination thereof. In embodiments, expressing or increasing expression of the one or more immunoprotective proteins is under the control of a constitutively active promoter.

In embodiments, expressing or increasing expression of the one or more immunoprotective proteins is at the DNA level by one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA. In embodiments, expressing or increasing expression of the one or more immunoprotective proteins is at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA. In embodiments, expressing or overexpressing the one or more immunoprotective proteins is by one or more of a small regulatory RNA, miRNA, IRES element, transcription factor, or a combination thereof.

In embodiments, the hypoimmunogenic cell is allogenic. In embodiments, the hypoimmunogenic cell does not cause an immune reaction in patients to which it or a biological composition derived therefrom is administered.

In embodiments, the hypoimmunogenic cell comprises one or more targeting agents. In embodiments, the one or more targeting agents comprises a chimeric antigen receptor (CAR). In embodiments, the CAR is bispecific. In embodiments, the CAR lacks an intracellular portion. In embodiments, the CAR comprises a targeting agent, a transmembrane domain, and an intracellular domain, which comprises a costimulatory domain and/or a signaling domain. In embodiments, the transmembrane domain is derived from CD28, CD3ζ, CD4, CD8α, or ICOS, or a fragment thereof. In embodiments, the intracellular domain comprises an intracellular signaling domain of a CD3ζ-chain and/or one or more co-stimulatory molecules, optionally selected from CD28, 4-1BB, ICOS, CD27, and OX40.

In embodiments, the one or more targeting agents comprises an antibody or antibody format. In embodiments, the antibody or antibody format is selected from one or more of a monoclonal antibody, polyclonal antibody, antibody fragment, Fab, Fab′, Fab′-SH, F(ab′)2, Fv, single chain Fv (scFv), V_{NAR}, VHH, affilin, diabody, nanobody, linear antibody, bispecific antibody, multi-specific antibody, chimeric antibody, humanized antibody, human antibody, and fusion protein comprising the antigen-binding portion of an antibody. In embodiments, the antibody format is a scFv. In embodiments, the one or more targeting agents comprises a viral epitope recognition receptor (VERR) or viral ligand. In embodiments, the one or more targeting agents comprises a ligand for a receptor. In embodiments, the one or more targeting agents comprises a receptor for a ligand.

In embodiments, the one or more targeting agents is operably linked to a regulatable expression element.

In embodiments, described herein is a hypoimmunogenic cell produced by any methods described herein. In embodiments, described herein is a pharmaceutical composition comprising a hypo immunogenic cell produced by any methods described herein, and one or more excipients.

In embodiments, described herein is a hypoimmunogenic cell comprising (a) one or more membrane-embedded targeting agents targeted against one or more cellular biomarkers, (b) expression or increased expression of immunoprotective proteins comprising (i) α-phagocytic integrin, CCL2, H2-M3, MFG-E8, and FasL, wherein FasL is not overexpressed, (ii) PD-L1 and/or CTLA-4, wherein the hypoimmunogenic cell is not activated with the expression of PD-L1, and (iii) one of either CD24, CD47, CD200, chimeric CD24/CD47, chimeric CD24/CD200, and chimeric CD47/CD200, or two of either CD24, CD47, and CD200, and (c) substantially lacking expression and/or activity of immunogenic proteins comprising (i) HLA-A, HLA-B, HLA-C, HLA-F, CIITA, PD-1, IL-6, T cell alpha chain (TRAC), and T cell beta chain (TRBC), and (ii) HLA-E or HLA-G.

In embodiments, described herein is a hypoimmunogenic cell comprising (a) one or more membrane-embedded targeting agents targeted against one or more cellular biomarkers, (b) expression or increased expression of immunoprotective proteins comprising (i) α-phagocytic integrin, CCL2, H2-M3, MFG-E8, and FasL, wherein FasL is not overexpressed, (ii) PD-L1 and/or CTLA-4, wherein the hypoimmunogenic cell is not activated with the expression of PD-L1, (iii) either CD24 and CD47, or a chimeric CD24/CD47, and (c) substantially lacking expression and/or activity of immunogenic proteins comprising (i) HLA-A, HLA-B, HLA-C, HLA-F, CIITA, PD-1, SerpinB9, IL-6, T cell alpha chain (TRAC), and/or T cell beta chain (TRBC), and (ii) HLA-E or HLA-G.

In embodiments, the hypoimmunogenic cell is substantially lacking expression and/or activity of one or more of IL-4, IL-10, and/or IL-16. In embodiments, the hypoimmunogenic cell further comprises expression or increased expression of an antibody or antibody format molecule which targets an IL-6 surface receptor (anti-IL-6R). In embodiments, the hypoimmunogenic cell further comprises expression or increased expression of NCAM.

In embodiments, the one or more targeting agents comprises a chimeric antigen receptor (CAR). In embodiments, the one or more targeting agents are an antibody or antibody format. In embodiments, the one or more targeting agents are an antibody or antibody format. In embodiments, the antibody or antibody format is selected from one or more of a monoclonal antibody, polyclonal antibody, antibody fragment, Fab, Fab′, Fab′-SH, F(ab′)2, Fv, single chain Fv (scFv), V_{NAR}, V_{H}H, afflilin, diabody, nanobody, linear antibody, bispecific antibody, multi-specific antibody, chimeric antibody, humanized antibody, human antibody, and fusion protein comprising the antigen-binding portion of an antibody. In embodiments, the one or more targeting agents is a viral epitope recognition receptor (VERR) or viral ligand. In embodiments, the one or more targeting agents is a ligand for a receptor or a receptor for a ligand.

In embodiments, the hypoimmunogenic cell is allogenic. In embodiments, the hypoimmunogenic cell does not cause an immune reaction in patients to which it or a biological composition derived therefrom is administered.

### BRIEF DESCRIPTION OF THE DRAWINGS

FIG. 1 depicts a non-limiting diagrammatic representation of T cell activation via the interaction between the MHC class I T cell receptor (TCR) synapse. Target peptide display via MHC class I complexes on virally infected cells, cancer cells, etc., can be recognized by hypervariable regions of TCRα/β on lymphocytes, resulting in conformational changes of the intracellular domains that trigger signaling to produce cytotoxic cytokines.

FIG. 2 depicts a non-limiting diagrammatic representation of an activated immune cell containing anti-cancer cytokines processed into a biomimetic nanovesicle (BioNV). Anti-cytokines (and various cytotoxic peptides) can be loaded into BioNVs which can be used for robust delivery.

FIG. 3 depicts a non-limiting diagrammatic representation of a process of activating cells with a biomarker antigen via its CAR/TCR receptor. The biomarker antigen can be conjugated to a magnetic or streptavidin bead (or others), then added to the cellular suspension to cause activation. After activation, the antigen can be separated from the cellular suspension, leaving the activated cell with an unbound CAR/TCR complex.

FIG. 4 depicts a graphical representation of a B2M genetic knock-out strategy for hypoimmunogenic cell line development from human fibroblast reprogrammed iPSCs. β2-macroglobulin (B2M) is a serum protein found in association with the major histocompatibility complex (MHC) class I heavy chain on the surface of nearly all nucleated cells involved in the presentation of peptide antigens to the immune system.

FIG. 5 depicts a graphical representation of a B2M gRNA design for hypoimmunogenic cell line development. The strategy, in embodiments, involves using CRISPR/Cas9 and a specifically designed gRNA to target relevant regions of the B2M gene.

FIG. 6 depicts a tabular representation of a B2M gRNA off-target analysis for hypoimmunogenic cell line development.

FIG. 7 depicts a graphical representation of a CIITA genetic knock-out strategy for hypoimmunogenic cell line development from human fibroblast reprogrammed iPSCs. Master Control Factor CIITA is a MHC class II Trans-activator that is involved in the transcriptional regulation of all MHC II genes.

FIG. 8 depicts a graphical representation of a CIITA gRNA design for hypoimmunogenic cell line development. The strategy, in embodiments, involves using CRISPR/Cas9 and a specifically designed gRNA to target relevant regions of the CIITA gene.

FIG. 9 depicts a tabular representation of a CIITA gRNA off-target analysis for hypoimmunogenic cell line development.

FIG. 10 depicts a graphical representation of a B2M knock-out clonal sequence analysis for hypoimmunogenic cell line development from human fibroblast reprogrammed iPSCs. The results for two selected B2M/CIITA double knock-out clones are shown. Sequencing data illustrate successful B2M KO in the double KO clones.

FIG. 11 depicts a graphical representation of a CIITA knock-out clonal sequence analysis for hypoimmunogenic cell line development from human fibroblast reprogrammed iPSCs. The results for two selected B2M/CIITA double knock-out clones are shown. Sequencing data illustrate successful CIITA KO in the double KO clones.

FIG. 12 depicts a graphical representation of the B2M/CIITA double knock-out clonal sequence summary and clonal morphology in vitro human fibroblast reprogrammed iPSCs hypoimmunogenic cells. The data illustrate that the clonal populations resulted in different overall knock-outs.

FIG. 13 depicts a graphical representation of a human CD47 (hCD47) isoform 2 knock-in for hypoimmunogenic cell line development from human fibroblast reprogrammed iPSCs. A pcDNA3.1(+)XCC92 mammalian vector (6271 bp length) is used with selection markers. Deletion of the CD47 3′UTR is performed for stable clonal surface expression of CD47. The 3′UTR contains at least 6 microRNA binding sites that repress CD47 expression, where a bGH poly A tail used in its place.

FIG. 14 depicts a graphical representation of hCD47 isoform 2 knock-in clonal selection for hypoimmunogenic cell line development in B2M/CIITA double KO human fibroblast reprogrammed iPSCs. Data indicate successful hCD47 KI in the double KO cell line.

FIG. 15 depicts a graphical representation of a TRAC genetic knock-out strategy for hypoimmunogenic cell line development from human fibroblast reprogrammed iPSCs. In embodiments, the T cell receptor alpha chain (TRAC) elimination is done to prevent interference with CAR targeting and off-target effects.

FIG. 16 depicts a graphical representation of a TRAC gRNA design for hypoimmunogenic cell line development. The strategy, in embodiments, involves using CRISPR/Cas9 and a specifically designed gRNA to target relevant regions of the TRAC gene.

FIG. 17 depicts a tabular representation of a TRAC gRNA off-target analysis for hypoimmunogenic cell line development.

FIG. 18 depicts a graphical representation of a TRBC1 genetic knock-out strategy for hypoimmunogenic cell line development from human fibroblast reprogrammed iPSCs. In embodiments, the T cell receptor beta chain 1 (TRBC1) elimination is done to prevent interference with CAR targeting and off-target effects.

FIG. 19 depicts a graphical representation of a TRBC1 gRNA design for hypoimmunogenic cell line development. The strategy, in embodiments, involves using CRISPR/Cas9 and a specifically designed gRNA to target relevant regions of the TRBC1 gene.

FIG. 20 depicts a tabular representation of a TRBC1 gRNA off-target analysis for hypoimmunogenic cell line development.

FIG. 21 depicts a graphical representation of a TRAC knock-out clonal sequence analysis for hypoimmunogenic cell line development for the B2M/CIITA double knock-out, hCD47 knock-in in human fibroblast reprogrammed iPSCs. The results for two selected clones are shown. Sequencing data illustrate successful TRAC knock-out in the double KO, CD47 KI clones.

FIG. 22 depicts a graphical representation of a TRBC1 knock-out clonal sequence analysis for hypoimmunogenic cell line development for the B2M/CIITA double knock-out, hCD47 knock-in, and TRAC knock-out in human fibroblast reprogrammed iPSCs. The results for two selected clones are shown. Sequencing data illustrate successful TRBC1 knock-out in the double KO, CD47 KI, TRAC KO clones.

FIG. 23 depicts a graphical representation of the B2M/CIITA double knock-out, TRAC/TRBC1 double knock-out, hCD47 KI human fibroblast reprogrammed iPSCs hypoimmunogenic cells. The data illustrate the clonal in vitro morphology analysis.

FIG. 24 depicts a graphical representation of an IL-6 genetic knock-out strategy for hypoimmunogenic cell line development from human fibroblast reprogrammed iPSCs. Interleukin-6 (IL-6) elimination is to precent Cytokine Release Syndrome (CRS).

FIG. 25 depicts a graphical representation of an IL-6 gRNA design for hypoimmunogenic cell line development. The strategy, in embodiments, involves using CRISPR/Cas9 and a specifically designed gRNA to target relevant regions of the IL-6 gene.

FIG. 26 depicts a tabular representation of an IL-6 gRNA off-target analysis for hypoimmunogenic cell line development.

FIG. 27 depicts a non-limiting diagrammatic representation of producing BioNVs using serial extrusion.

### DETAILED DESCRIPTION

The present disclosure relates to, in part, methods of generating a hypoimmunogenic cell which are used for generating cell-derived biomimetic nanovesicles (BioNVs). BioNVs circumvent the caveats of whole cell therapies, while retaining the functionality of the cells they are derived from. Although manufacturing costs are lower with nanovesicles (NVs) compared to whole cell therapies, NV therapeutics can suffer from half-life and elimination issues, for example, being cleared by immune cells and the kidneys. The methods of Harding et al., Deuse et al., and Zhao et al. have direct implications in whole cell therapeutics and partially achieve hypoimmunogenicity, allowing for a foundation for truly “off-the-shelf” therapeutics, but are not useful for BioNV formation (Harding et al., “Induction of long-term allogeneic cell acceptance and formation of immune privileged tissue in immunocompetent hosts.” BioRxiv 716571 [Preprint], Jul. 30, 2019. doi: 10.1101/716571.), (Deuse et al. “Hypoimmunogenic derivatives of induced pluripotent stem cells evade immune rejection in fully immunocompetent allogeneic recipients.” Nat. Biotechnol. Vol. 37, No. 3, 2019: pp. 252-258. doi: 10.1038/s41587-019-0016-3), and (Zhao W, et al. “Strategies for Genetically Engineering Hypoimmunogenic Universal Pluripotent Stem Cells.” iScience. Vol. 23, No. 6, 2020:101162. doi: 10.1016/j.isci.2020.101162.).

In embodiments, methods of generating hypoimmunogenic cells which are used to produce BioNVs that overcome the shortcomings of other cells used for BioNVs, for example, 1) the expression of SerpinB9 inhibits granzyme function, thereby limiting the functionality of the BioNV mechanism of action; 2) the overexpression of FasL is not necessary due to the increase in naturally expressed FasL density on the surface of the BioNV that occurs during cellular processing (e.g., after extrusion); 3) the overexpression of CD200 may inhibit granulocyte function that is necessary for efficacy in the solid tumor microenvironment (if used for cancer treatment purposes); 4) the overexpression of too many anti-phagocytic tags can contribute to cell products that are too stable and difficult to clear from the body after therapeutic efficacy.

In embodiments, methods of generating hypoimmunogenic cells improve upon other methods of generating hypoimmunogenic cells, which focus on reducing or ablating the expression and/or activity (e.g., knocking-out) the MHC class I/II genes and/or HLA genes followed by the overexpression of CD47. For example, the overexpression of CD47 can be inhibitory to BioNV function for several reasons. (1) Knocking out the B2M and CIITA genes in combination with the overexpression of a CD47 tag may dampen the desired NK cell response at the target site of the BioNV. This requirement is generally not sought for whole cell therapies, but BioNVs lack the genetic material that contributes to the triggers of NK cell responses to cells lacking all HLA genes. (2) The overexpression of CD47 in some differentiated cell subsets could be inhibited, thereby reliance of CD47 limits the types of cells that could be used for BioNVs. CD47 is a highly regulated surface protein due to its importance for cell stability. If CD47 expression is too low, the cells (and BioNVs) run the risk of premature immune clearance. Conversely, if CD47 expression is too high, (such as happens in cancer cells) the cells may not be cleared by the immune system in a timely manner. The expression of CD47 isoform 2 gene is tightly regulated at the levels of transcription (via several transcription factors including STAT3, NF-kβ, Hif-1, Myc), RNA translation by the microRNAs (miR-708, miR-192, miR-222, miR-133a, miR-155 miR-200a and miR-340), and post-translational protein modifications (formation of N-terminal pyroglutamate that aids in interactions with SIRPα). The tight regulation of CD47 within the cellular environment can become inhibitory for its overexpression purposes as a protective anti-phagocytic tag in cells that are differentiated away from the engineered iPSC source, even if the iPSCs are derived from syncytiotrophoblast cells where CD47 is naturally overexpressed (i.e., lineage differentiation can result in down regulation). (3) The TCR genes remain intact. In iPSCs differentiated to T cell (naïve) lineages, the presence of TCRs can contribute to GVHD in recipient patients. Numerous studies have reported the occurrence of TCR-mediated non-MHC antigen rejections in patients.

In embodiments, methods for generating a hypoimmunogenic cell line which is engineered to be allogeneic and not cause an adverse immune reaction in patients. The hypoimmunogenic cell can be engineered to express a CAR, among another targeting agent formats (e.g., VERR, V_{NAR}, VHH, scFv, bispecific T cell engager (BiTE), engineered T cell receptor (TCR), dual-affinity retargeting (DART) antibody, etc.). The hypoimmunogenic cell can retain the ability to be activated to express a variety of intracellular therapeutic biomolecules (e.g., cytokines, perforin, granzyme, interferons (INFs), tumor necrosis factors (TNFs), interleukins (ILs), etc.) which can be packaged into a BioNV. The hypoimmunogenic cell can be engineered to produce gene editing payloads, including nucleic acids (e.g., plasmids and gene cassettes expressing tracer RNA (trRNA), guide RNA (gRNA), miRNA, tRNA, RNAi, small RNAs, and endonucleases, e.g., CRISPR, TALEN, ZFNs, etc.), among other gene editing formats that can be packaged into the resultant BioNV. The hypoimmunogenic cell can be engineered to produce and package therapeutic fusion proteins, antibodies and antibody fragments, nucleic acids, etc., which can be packaged into BioNVs, or naturally shed as exosomes. The hypoimmunogenic cell can be differentiated while retaining its properties, for example, to a T cell, macrophage, cardiomyocyte, etc., which can then confer new properties to the cell, such as the ability to cross biological barriers, recruit cells, etc. BioNVs derived from the hypoimmunogenic cells described herein can retain these properties and payloads of the cell, representing an effective alternative to whole cell therapies.

In embodiments, described herein is a hypoimmunogenic cell. In embodiments, described herein are hypoimmunogenic cells produced using the present methods.

The hypoimmunogenic cell can be modified to express one or more targeting agents (e.g., a CAR) targeted against one or more cellular biomarkers. The hypoimmunogenic cell can be modified to express or have increased expression of one or more membrane-embedded immunoprotective surface markers, including α-phagocytic integrins, CCL2, PD-1, CTLA-4, H2-M3, SerpinB9, CD24, CD47, a chimeric form of CD24/CD47, CD200, a CD200 chimera with either CD24 or CD47, MFG-E8, anti-IL-6R, NCAM, and/or FasL. The hypoimmunogenic cell can be modified to substantially lack expression and/or activity of one or more immunogenic proteins, including MHC class I, MHC class II, HLA-A, HLA-B, HLA-C, HLA-E or HLA-G, HLA-F, CIITA, PD-1, SerpinB9, IL-4, IL-6, IL-10, IL-16, T cell alpha chain (TRAC), and/or T cell beta chain (TRBC). The hypoimmunogenic cell can express or not express CD200 and/or SerpinB9 depending on the functionality of the desired BioNV to be derived therefrom, for example, for delivery of granzyme.

In embodiments, “increased expression and/or activity,” as used herein refers to an increase in expression and/or activity in the hypoimmunogenic cell in comparison to its native, or wild-type cognate cell. For example, in embodiments, the increased expression and/or activity of one or more biomolecules described herein can confer the hypoimmunogenic properties of an iPSC relative to an iPSC which does not have the same expression pattern or expression level of the protein. In embodiments, the “increased expression and/or activity,” is due to a genetic amendment, such as a knock-in.

In embodiments, described herein are compositions of hypoimmunogenic cells and kits comprising hypoimmunogenic cells. Compositions can include whole cells, suspended in a solution compatible with cryopreservation of the cells or administration of the cells to a subject (e.g., intravenous, intraperitoneal, intramuscular, etc.). Hypoimmunogenic cell compositions can include additional therapeutic agents for use as a whole cell therapy. Kits can include any composition of hypoimmunogenic cells described herein, for example, packaged into a syringe, IV bag, and include instructions for use and diagnostic materials.

### Methods of Generating Hypoimmunogenic Cell Lines

In aspects, the present disclosure includes methods of generating a hypoimmunogenic cell comprising reducing or ablating the expression and/or activity of one or more immunogenic proteins in a cell, and expressing or increasing expression and/or activity of one or more immunoprotective proteins in the cell, thereby generating the hypoimmunogenic cell.

In embodiments, the hypoimmunogenic cell can originate from any cell capable of differentiating into a specific cell type. In embodiments, the cell is a stem cell, an induced pluripotent stem cell (iPSC), a reprogrammed pluripotent or multipotent cell, an embryonic stem cell, a mesenchymal stem cell, or a differentiated cell which originates from any stem cell thereof. In embodiments, the differentiated cell is a lymphoid lineage cell, such as a T cell, helper T cell, T-memory cell, NK cell, etc. In embodiments, the differentiated cell is a myeloid lineage cell, such as a macrophage, monocyte, neutrophil, etc. In embodiments, the differentiated cell is a tissue-specific cell, such as a hepatocyte, cardiomyocyte, neuron, endothelial cell, pancreatic cell, retinal pigmented epithelium (RPE) cell, etc. In embodiments, the hypoimmunogenic cell can be any terminally differentiated cell, for example and without limitation, a muscle cell (satellite cell), adipocyte, osteocyte, cardiomyocyte, hepatocyte, blood cell (including erythrocyte, thrombocyte, and all immune cell types), glial cell (among other neuronal cell types), epithelial cell, epidermal cell, interstitial cell (e.g., respiratory interstitial cell), fibroblast (e.g., dermal fibroblast), endothelial cell (e.g., bronchial endothelial cell), oral cell, stromal cell, or germ cell. In embodiments, the hypoimmunogenic cell can be any function-specific cell type, for example and without limitation, exocrine secretory epithelial cell, hormone-secreting cell (e.g. enteroendocrine cell, thyroid cell, pancreatic islet cell, etc.), sensory transducer cell, autonomic neuronal cell, sensory organ cell (e.g., pillar cell, olfactory cell, Schwann cell, satellite glial cell, etc.), barrier cell (e.g., pneumocyte, duct cell, kidney cell, podocyte, etc.), extracellular matrix cell (e.g., tendon fibroblast, osteoblast, connective tissue cell, etc.), or contractile cell (e.g., skeletal muscle cell, cardiac muscle cell, myoepithelial cell, etc.).

In embodiments, the hypoimmunogenic cell (or the cell used to generate the hypoimmunogenic cell) is a human female cell (e.g., from a female donor or source). In embodiments, the hypoimmunogenic cell (or the cell used to generate the hypoimmunogenic cell) is a human male cell (e.g., from a male donor or source). In embodiments the hypoimmunogenic cell (or the cell used to generate the hypoimmunogenic cell) is a human female fibroblast iPSC. In embodiments, the hypoimmunogenic cell (or the cell used to generate the hypoimmunogenic cell) is a human male fibroblast iPSC.

In embodiments, the allogeneic and hypoimmunogenic properties of the cells (e.g., derived from iPSCs) are created by knocking-out, silencing, inactivating, blocking or otherwise negating the expression, transcriptional efficiencies, and/or activity of one or more immunogenic molecules. In embodiments, reducing or ablating the expression and/or activity of one or more immunogenic proteins comprises a 32-macroglobulin (B2M) gene disruption and/or a disruption that reduces or ablates MHC class I protein expression and/or activity, such as in the case for CD8+ T cell lineages. In embodiments, the reducing or ablating the expression and/or activity of one or more immunogenic proteins comprises a CIITA gene disruption and/or a disruption that reduces or ablates MHC class II protein expression and/or activity, such as in the case of CD4+ T cell lineages. Without wishing to be bound by theory, these proteins contribute to the human leukocyte antigen (HLA) immunogenicity that requires HLA allele matching in the donor-recipient for treatment by cell-based therapies. In embodiments, allogeneic and/or hypoimmunogenic properties are achieved by reducing or ablating the expression and/or activity of the genes encoding the T cell receptor (TCR) proteins including, for example, the a and B chains (as in the case of αβ T cells) or the γ and δ chains (as in the case of γδ T cells) forming the ligand-binding site and the signaling modules CD3δ, CD3γ, CD3ε, and CD3ζ. In embodiments, this is performed to reduce extraneous T cell receptor types other than those of the CAR cassette and further improve the homogeneity of the CAR of interest and reduce off-target effects in BioNV formation.

In embodiments, the hypoimmunogenic cell is substantially lacking one or more of MHC class I protein complexes, MHC class II complexes, T cell receptor (TCR) complexes, and/or cytokine release syndrome (CRS) proteins. In embodiments, reducing or ablating the expression and/or activity of one or more immunogenic proteins comprises a 32-macroglobulin (B2M) gene disruption and/or a disruption that reduces or ablates MHC class I protein expression and/or activity. In embodiments, knocking-out the B2M genes can reduce the number of potential doses to be administered due to the risk of preventing long term acceptance of the BioNVs by the recipient, such as what has been observed in the whole cell-based approaches described above. To overcome this issue, in embodiments, the HLA-E or HLA-G gene remains intact, allowing the immune system to adapt to the resulting BioNV. In embodiments, the HLA-A, HLA-B, HLA-C, HLA-F, and HLA-E or HLA-G (but not both of HLA-E or HLA-G) are knocked out sequentially.

In embodiments, reducing or ablating the expression and/or activity of one or more immunogenic proteins comprises an HLA-A gene disruption and/or a disruption that reduces or ablates HLA-A protein expression and/or activity. In embodiments, reducing or ablating the expression and/or activity of one or more immunogenic proteins comprises an HLA-B gene disruption and/or a disruption that reduces or ablates HLA-B protein expression and/or activity. In embodiments, reducing or ablating the expression and/or activity of one or more immunogenic proteins comprises an HLA-C gene disruption and/or a disruption that reduces or ablates HLA-C protein expression and/or activity. In embodiments, reducing or ablating the expression and/or activity of one or more immunogenic proteins comprises an HLA-E gene disruption or an HLA-G gene disruption and/or a disruption that reduces or ablates HLA-E or HLA-G protein expression and/or activity. In embodiments, reducing or ablating the expression and/or activity of one or more immunogenic proteins comprises an HLA-F gene disruption and/or a disruption that reduces or ablates HLA-F protein expression and/or activity.

In embodiments, the hypoimmunogenic cell comprises a CIITA gene disruption and/or a disruption that prevents MHC class II protein expression. In embodiments, allogeneic iPSCs have their CIITA gene disrupted, so that a resulting differentiated cell line (e.g., DCs, mononuclear phagocytes, endothelial cells, thymic epithelial cells, B cells, etc.) does not express or has reduced expression of MHC class II proteins.

In embodiments, reducing or ablating the expression and/or activity of one or more immunogenic proteins comprises a T cell alpha constant (TRAC) gene disruption and/or a disruption that reduces or ablates TRAC protein expression and/or activity. In embodiments, reducing or ablating the expression and/or activity of one or more immunogenic proteins comprises a T cell beta constant (TRBC) gene disruption and/or a disruption that reduces or ablates TRBC protein expression and/or activity. In embodiments, reducing or ablating the expression and/or activity of one or more immunogenic proteins comprises a PD-1 gene disruption and/or a disruption that reduces or ablates PD-1 protein expression and/or activity.

CRS is a major concern with whole cell therapies, where despite engineered hypoimmunogenicity, effector functions and other consequences of interaction with cells post-infusion can result in the release of biomolecules that result in a systemic inflammatory syndrome characterized by fever, multiple organ dysfunction, etc. In embodiments, the hypoimmunogenic cell is engineered to disrupt one or more proteins that contribute to CRS. In embodiments, the hypoimmunogenic cell has reduced or ablated expression and/or activity (e.g., knock-out or silencing) of CRS-related cytokines.

In embodiments, reducing or ablating the expression and/or activity of one or more immunogenic proteins comprises an IL-4 gene disruption and/or a disruption that reduces or ablates IL-4 protein expression and/or activity. In embodiments, reducing or ablating the expression and/or activity of one or more immunogenic proteins comprises an IL-6 gene disruption and/or a disruption that reduces or ablates IL-6 protein expression and/or activity. In embodiments, IL-6 knock-out prevents undesirable IL-6 packaging into the BioNV and reduces the BioNV's contribution to a localized (and concentrated due to biomarker targeting) and/or potentially systemic CRS events. In embodiments, reducing or ablating the expression and/or activity of one or more immunogenic proteins comprises an IL-10 gene disruption and/or a disruption that reduces or ablates IL-10 protein expression and/or activity. In embodiments, reducing or ablating the expression and/or activity of one or more immunogenic proteins comprises an IL-16 gene disruption and/or a disruption that reduces or ablates IL-16 protein expression and/or activity. In embodiments, the reduction or ablation of interleukins decreases the likelihood of CRS.

Serine proteinase inhibitor B9 (SerpinB9) is a member of the serine protease inhibitor superfamily. SerpinB9 has been reported to protect cells from the immune-killing effects of granzyme B. In embodiments, the hypoimmunogenic cell expresses or has increased expression of SerpinB9. In embodiments, the hypoimmunogenic cell has SerpinB9 knocked-out and/or silenced. In embodiments, reducing or ablating the expression and/or activity of one or more immunogenic proteins comprises a SerpinB9 gene disruption and/or a disruption that reduces or ablates SerpinB9 protein expression activity.

In embodiments, the overexpression of SerpinB9 sequesters the function of granzyme B which is related to immunostimulatory responses, such as apoptosis of a targeted and/or diseased cell. In embodiments, granzyme B is inhibited in activated lymphocytes, NK cells, macrophages, and follicular DCs, among other cell types. In embodiments, e.g., where a BioNV that is intended to deliver a non-granzyme payload, for example a gene editing payload, the hypoimmunogenic cell can express and/or overexpress SerpinB9.

In embodiments, reducing or ablating the expression and/or activity of one or more immunogenic proteins is at the DNA level by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, small RNA, or a combination thereof. In embodiments the small RNA is or comprises one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA. In embodiments, reducing or ablating the expression and/or activity of one or more immunogenic proteins is at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

In embodiments, the site-specific nuclease used for any genetic modification herein can include CRISPR/Cas endonucleases, TALENS, ZFNs, or any other site-specific nuclease system for gene silencing and/or knock-out. In embodiments the gene disruption is due to gene-editing system, including one or more proteins and/or nucleic acids working in concert, for example as in TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog of any of these editors.

In embodiments, gRNA sequences compatible with a CRISPR/Cas9 system can be designed to be targeted against at least a portion of the B2M gene (including coding and non-coding sequences controlling gene expression) (e.g., as described in Table 8). For example, in non-limiting embodiments, SEQ ID NOs: 22-23 lists exemplary B2M gene sequences for designing gRNAs for knock-out purposes. In non-limiting embodiments, a series of illustrative gRNA sequences (listed in DNA format) are described in SEQ ID NOs: 21, 24, 26-29, and 133-134 targeted against sequences in SEQ ID NOs: 22-23. In non-limiting embodiments, such gRNAs can generate allelic knock-outs of B2M (including, e.g., biallelic knock-outs), resulting in clonal DNA sequences, for example as listed in SEQ ID NOs: 43-49. In embodiments, the resultant genomic DNA sequences from CRISPR/Cas modification B2M can be used to insert one or more genes, coding sequences, DNA sequences, etc., (for example within the space(s) flanked by SEQ ID NOs: 43-49 for B2M knock-out).

In embodiments, gRNA sequences compatible with a CRISPR/Cas9 system can be designed to be targeted against at least a portion of the CIITA gene (including coding and non-coding sequences controlling gene expression) (e.g., as described in Table 8). For example, in non-limiting embodiments, SEQ ID NOs: 32-33 lists exemplary CIITA gene sequences for designing gRNAs for knock-out purposes. In non-limiting embodiments, a series of illustrative gRNA sequences (listed in DNA format) are described in SEQ ID NOs: 31, 34, and 36-42 targeted against sequences in SEQ ID NOs: 32-33. In non-limiting embodiments, such gRNAs can generate allelic knock-outs of CIITA (including, e.g., biallelic knock-outs), resulting in clonal DNA sequences, for example as listed in SEQ ID NOs: 50-55. In embodiments, the resultant genomic DNA sequences from CRISPR/Cas modification of CIITA can be used to insert one or more genes, coding sequences, DNA sequences, etc., (for example within the space(s) flanked by SEQ ID NOs: 50-55 for CIITA knock-out).

In embodiments, gRNA sequences compatible with a CRISPR/Cas9 system can be designed to be targeted against at least a portion of the TRAC gene (including coding and non-coding sequences controlling gene expression) (e.g., as described in Table 8). For example, in non-limiting embodiments, SEQ ID NOs: 56-57 lists exemplary TRAC gene sequences for designing gRNAs for knock-out purposes. In non-limiting embodiments, a series of illustrative gRNA sequences (listed in DNA format) are described in SEQ ID NOs: 60-75 targeted against sequences in SEQ ID NOs: 56-57. In non-limiting embodiments, such gRNAs can generate allelic knock-outs of TRAC (including, e.g., biallelic knock-outs), resulting in clonal DNA sequences, for example as listed in SEQ ID NOs: 102-108. In embodiments, the resultant genomic DNA sequences from CRISPR/Cas modification of TRAC can be used to insert one or more genes, coding sequences, DNA sequences, etc., (for example within one or more space(s) flanked by SEQ ID NOs: 102-108 for TRAC knock-out).

In embodiments, gRNA sequences compatible with a CRISPR/Cas9 system can be designed to be targeted against at least a portion of the TRBC1 gene (including coding and non-coding sequences controlling gene expression) (e.g., as described in Table 8). For example, in non-limiting embodiments, SEQ ID NOs: 76-77 lists exemplary TRBC1 gene sequences for designing gRNAs for knock-out purposes. In non-limiting embodiments, a series of illustrative gRNA sequences (listed in DNA format) are described in SEQ ID NOs: 79-101 targeted against sequences in SEQ ID NOs: 76-77. In non-limiting embodiments, such gRNAs can generate allelic knock-outs of TRBC1 (including, e.g., biallelic knock-outs), resulting in clonal DNA sequences, for example as listed in SEQ ID NOs: 109-110 and 112-116. In embodiments, the resultant genomic DNA sequences from CRISPR/Cas modification of TRBC1 can be used to insert one or more genes, coding sequences, DNA sequences, etc., (for example within one or more space(s) flanked by SEQ ID NOs: 109-110 and 112-116 for TRBC1 knock-out).

In embodiments, gRNA sequences compatible with a CRISPR/Cas9 system can be designed to be targeted against at least a portion of the IL-6 gene (including coding and non-coding sequences controlling gene expression) (e.g., as described in Table 8). For example, in non-limiting embodiments, SEQ ID NOs: 117-118 lists exemplary IL-6 gene sequences for designing gRNAs for knock-out purposes. In non-limiting embodiments, a series of illustrative gRNA sequences (listed in DNA format) are described in SEQ ID NOs: 119-130 targeted against sequences in SEQ ID NOs: 117-118. In non-limiting embodiments, such gRNAs can generate allelic knock-outs of IL-6 (including, e.g., biallelic knock-outs), resulting in clonal DNA sequences. In embodiments, the resultant genomic DNA sequences from CRISPR/Cas modification of IL-6 can be used to insert one or more genes, coding sequences, DNA sequences, etc.

In embodiments, hypoimmunogenic cells can be generated by knocking-out one or more genes described herein using one or more gRNAs as described herein. For example, in embodiments, the gRNA comprises about or at least about 85% sequence identity, about or at least about 86% sequence identity, about or at least about 87% sequence identity, about or at least about 89% sequence identity, about or at least about 90% sequence identity, about or at least about 91% sequence identity, about or at least about 92% sequence identity, about or at least about 93% sequence identity, about or at least about 94% sequence identity, about or at least about 95% sequence identity, about or at least about 96% sequence identity, about or at least about 97% sequence identity, about or at least about 98% sequence identity, or about or at least about 99% sequence identity to one or more of SEQ ID NOs: 31, 24, 26-29, 31, 34, 36-42, 60-75, 79-101, 119-130, and 133-134.

In embodiments, hypoimmunogenic cells can be generated by targeting one or more genes described herein for knock-out and/or for inserting one or more proteins for expression and/or increased expression and/or activity by targeting one or more DNA sequences, as described herein. For example, in embodiments, the DNA sequence comprises one or more of SEQ ID NOs: 22-23, 32-33, 56-57, 76-77, and 117-118, including sequences having one or more of a substitution, deletion, insertion, variant, and/or nucleotide polymorphism (e.g., single nucleotide polymorphism SNP) therein.

In embodiments, the hypoimmunogenic cell (and BioNV derived therefrom) expresses or has increased expression of one or more immunoprotective proteins. In embodiments, expressing or increasing expression and/or activity of one or more immunoprotective proteins comprises expressing or increasing expression of a CD34 gene and/or gene product. In embodiments, expressing or increasing expression and/or activity of one or more immunoprotective proteins comprises expressing or increasing expression of a CCL2 gene and/or gene product. In embodiments, expressing or increasing expression and/or activity of one or more immunoprotective proteins comprises expressing or increasing expression of a PD-L1 gene and/or gene product, wherein the hypoimmunogenic cell is not activated with the expression of PD-L1. In embodiments, expressing or increasing expression and/or activity of one or more immunoprotective proteins comprises expressing or increasing expression of a H2-M3 gene and/or gene product.

In embodiments, expressing or increasing expression and/or activity of one or more immunoprotective proteins comprises expressing or increasing expression of a CD47 gene and/or gene product. In embodiments, prevention of the potential inhibitory phenotypes of CD47 expression across cells is done via interference with the inhibitory mechanism of action of the series of microRNAs on the 3′UTR of the CD47 gene by deleting this region in stable constructs or by eliminating/inhibiting the expression of the microRNAs. In embodiments, this can resolve inhibitory issues caused by the microRNAs.

In embodiments, expressing or increasing expression and/or activity of one or more immunoprotective proteins comprises expressing or increasing expression of a CD24 gene and/or gene product. CD24 is a sialoglycoprotein expressed on mature granulocytes and B-cells and is also an anti-phagocytic protein. CD24 prevents phagocytosis through interactions with Siglec-G/10 on macrophages. In embodiments, the hypoimmunogenic cell expresses or overexpresses a CD24 protein and/or gene product. In embodiments, expressing or increasing expression and/or activity of one or more immunoprotective proteins comprises expressing or increasing expression of a chimeric CD24/CD47 gene and/or gene product. In embodiments, the hypoimmunogenic cells include CD24/CD47, with a tethered transmembrane domain. In embodiments, the domains of CD47 isoform 2 and CD24 can be either separately expressed or tethered to form a bilobed, chimeric protein. In embodiments, the hypoimmunogenic cells are iPSCs are from fibroblasts, not from ABO cells.

In embodiments, expressing or increasing expression and/or activity of one or more immunoprotective proteins comprises expressing or increasing expression of a CD200 gene and/or gene product. In embodiments, CD200 tags minimize phagocytosis by macrophages and also prevent the activation of granulocytes. In embodiments, the hypoimmunogenic cell does not express CD200 when it is not desirable to prevent granulocytes, for example in the solid tumor microenvironment (TME), as activation of granulocytes would complement the mechanism of action of a BioNV designed to release granzymes and perforins. However, in embodiments, if a CD47 or CD24 tag is used, or a CD24/CD47 chimeric, bilobed protein tag (each prevents phagocytosis) in combination with overexpressed H2-M3 (which dampens the NK response) is used, stability can be achieved without CD200, while allowing BioNV clearance. In embodiments, where granzymes and perforins are not a therapeutic biomolecule of choice, CD200 can be expressed to prevent the activation of granulocytes, while eliminating a CD47 tag or a CD24 tag, but not both tags. In embodiments, expressing or increasing expression and/or activity of one or more immunoprotective proteins comprises expressing or increasing expression of a chimeric CD24/CD200 gene and/or gene product or a chimeric CD47/CD200 gene and/or gene product. In embodiments, these CD200 strategies represent a hypoimmunogenic cell line for generating BioNVs for targeting non-cancer cells, such as targeting the liver, kidney, cardiac cells, and/or tissue regeneration pathways.

In embodiments, the hypoimmunogenic cell (or differentiated cell therefrom) does not express and/or overexpress all three of CD47, CD24, and CD200. In embodiments, the hypoimmunogenic cell is engineered such that BioNVs that result from the hypoimmunogenic cell line are stabilized, but not to a degree where the BioNVs are resistant to being cleared from the body. A BioNV that is too stable could eventually trigger a humoral response, resulting in limiting the number of doses or treatments that can be administered.

In embodiments, expressing or increasing expression and/or activity of one or more immunoprotective proteins comprises expressing or increasing expression of a CTLA-4 gene and/or gene product. In embodiments, expressing or increasing expression and/or activity of one or more immunoprotective proteins comprises expressing or increasing expression of an MFG-E8 gene and/or gene product. In embodiments, expressing or increasing expression and/or activity of one or more immunoprotective proteins comprises expressing or increasing expression of a NCAM gene and/or gene product. In embodiments, expressing or increasing expression and/or activity of one or more immunoprotective proteins comprises expressing or increasing expression of an α-phagocytic integrin gene and/or gene product. In embodiments, expressing or increasing expression and/or activity of one or more immunoprotective proteins comprises expressing or increasing expression of an antibody or antibody format molecule which targets an IL-6 surface receptor (anti-IL-6R).

In embodiments, expressing or increasing expression and/or activity of one or more immunoprotective proteins comprises expression of a FasL gene and/or gene product. In embodiments, expressing or increasing expression and/or activity of one or more immunoprotective proteins does not comprise overexpression of a FasL gene and/or gene product. In embodiments, the overexpression of FasL is avoided because an enrichment of naturally expressed levels of FasL is observed in the membranes of BioNVs after processing, e.g., via serial extrusion. Too high of concentrations of FasL can be counter-productive and prevent the recruitment of T-cells to the solid tumor and/or cause premature T-cell death.

In embodiments, the hypoimmunogenic cells can express one or more fusion proteins of one or more portions of any immunoprotective protein herein. For example, in embodiments, constructs can be made where the appropriate portion of a ligand of choice is tethered to a transmembrane domain. In embodiments, constructs can be made where the biologically relevant portion of two or more proteins are tethered together and/or to a transmembrane domain.

In embodiments, the hypoimmunogenic cell has reduced or ablated expression and/or activity of one or more immunogenic proteins, such as proteins that result in an immune response in the subject, donor-recipient mismatch, HLA alloimmunity, inflammation, CRS, and the like, such as MHC class I proteins, MHC class II proteins, HLA proteins, TCR proteins, CRS proteins, etc. In embodiments, the hypoimmunogenic cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

In embodiments, the modified cell has expression or increased expression and/or activity of one or more immunoprotective proteins, such as proteins that result prevent or reduce an immune response in the subject, prevent or reduce premature clearance of the BioNV in the subject, prevent or reduce phagocytosis, confer barrier-crossing functionality, and the like, such as CD47, CD24, CD200, CD34, CCL2, H2-M3, MFG-E8, PD-L1, CTLA-4, etc. In embodiments, the hypoimmunogenic cell is not activated, or in an activated state, with expression of PD-L1. In embodiments, the hypoimmunogenic cell has expression or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the hypoimmunogenic cell has reduced or ablated of a gene and/or gene product of HLA-A, HLA-B, HLA-C, HLA-F, CIITA, IL-6, TRAC, TRBC, and one of either HLA-E or HLA-G.

In embodiments, the hypoimmunogenic cell has reduced or ablated of a gene and/or gene product of HLA-A, HLA-B, HLA-C, HLA-F, CIITA, IL-6, TRAC, TRBC, SerpinB9, and one of either HLA-E or HLA-G.

In embodiments, the hypoimmunogenic cell has reduced or ablated expression and/or activity of a gene and/or gene product of HLA-A, HLA-B, HLA-C, HLA-F, CIITA, IL-6, TRAC, TRBC, SerpinB9, one of either HLA-E or HLA-G, and one or more of IL-4, IL-10, and IL-16.

In embodiments, the hypoimmunogenic cell expresses or has increased expression of α-phagocytic integrin, CCL2, H2-M3, FasL, MFG-E8, and PD-L1 and/or CTLA-4, wherein the hypoimmunogenic cell does not overexpress FasL, and wherein the hypoimmunogenic cell is not activated with expression of PD-L1, and one of either CD24, CD47, CD200, chimeric CD24/CD47, chimeric CD24/CD200, and chimeric CD47/CD200, or two of either CD24, CD47, and CD200.

In embodiments, the hypoimmunogenic cell expresses or has increased expression of α-phagocytic integrin, CCL2, H2-M3, FasL, MFG-E8, SerpinB9, and PD-L1 and/or CTLA-4, wherein the hypoimmunogenic cell does not overexpress FasL, and wherein the hypoimmunogenic cell is not activated with expression of PD-L1, and one of either CD24, CD47, CD200, chimeric CD24/CD47, chimeric CD24/CD200, and chimeric CD47/CD200, or two of either CD24, CD47, and CD200.

In embodiments, the hypoimmunogenic cell expresses or has increased expression of a CD200 gene and/or gene product and does not express and/or substantially lacks either a CD24 or a CD47 gene and/or gene product. In embodiments, the hypoimmunogenic cell has no expression and/or activity of a SerpinB9 gene and/or gene product and a CD200 gene and/or gene product.

### Immune-Incompetent HLA Complexes

The hypoimmunogenic cell for generating BioNVs, in embodiments, expresses or has increased expression of one or more immune incompetent complexes. In embodiments, the BioNV has one or more one or more immune incompetent complexes. The one or more immune incompetent complexes, in embodiments, include one or more human leukocyte antigens (HLAs), that are inhibited from eliciting a T cell response when the complex interacts with one or more T cells. The immune incompetent complexes, in embodiments, include fusion proteins which are inhibited from activating T cells, but maintain structural integrity to allow some level of effector function.

The hypoimmunogenic cell for generating BioNVs, in embodiments, expresses or has increased expression of one or more T cell receptor (TCR) in place or, or in addition to, a CAR construct, as described herein.

Constructs for immune incompetent HLAs include, in embodiments, one or more nucleic acids encoding the immune incompetent HLAs. In embodiments, the complex includes, optionally in order from N-terminus to C-terminus, an amino acid sequence or nucleic acid sequence encoding an amino acid sequence which includes a peptide and at least a portion of a human HLA class 1 heavy chain sequence. In embodiments, the immune incompetent HLA is a fusion protein with a peptide that binds the HLA, interfering with one or more interactions the HLA molecule has with a cell surface complex on an immune cell. In embodiments, the peptide does not elicit a substantial T cell response when the peptide interacts with one or more T cells. In embodiments, the peptide is incapable of activating the one or more T cells (e.g., CD4+ T cells, CD8+ T cells, etc.). In embodiments, the peptide is capable of binding to a receptor of the one or more T cells, and the binding is insufficient to activate the one or more T cells. In embodiments, the peptide binds to one or more HLA binding groove domain residues of the human HLA class 1 heavy chain sequence. In embodiments, the peptide modulates a conformation of the human HLA class 1 heavy chain sequence. The conformation, in embodiments, prevents one or more T cells from binding to the human HLA class 1 heavy chain sequence.

MHC class I molecules typically bind peptides of about 8-10 amino acids in length, but can also bind non-canonical, longer peptides (e.g. more than about 13 amino acids in length). The peptide, in embodiments, is about or at least about 7 amino acids, 8 amino acids, 9 amino acids, 10 amino acids, 11 amino acids, 12 amino acids, 13 amino acids, 14 amino acids, or 15 or more amino acids in length. In some embodiments, the human HLA class 1 heavy chain sequence comprises one or more class 1 HLAS.

The human HLA class 1 heavy chain sequence, in embodiments, is HLA-A, HLA-B, HLA-C, or any combination thereof. In embodiments, the human HLA class 1 heavy chain sequence comprises multiple versions of HLA-A, HLA-B, HLA-C, or any combination thereof. In embodiments, the human HLA class 1 heavy chain sequence comprises the HLA-A, wherein the HLA-A is displaced between the HLA-B and the HLA-C.

The complex, in embodiments, has one or more linkers between the peptide and the human HLA class 1 heavy chain sequence. The one or more linkers, in embodiments, are configured to resist or reduce proteolytic cleavage. In embodiments, the peptide is coupled to the complex by a disulfide bond. In embodiments, the one or more linkers comprise a conformation configured to not block one or more killer-cell immunoglobulin-like receptor (KIR) binding sites on the human HLA class 1 heavy chain sequence.

The complex, in embodiments, is a fusion protein with one or more immune checkpoint agonists. In embodiments, the one or more immune checkpoint agonists include CD47, PD-L1, PD-L2, A2AR, B7-H3, B7-H4, BTLA, CTLA-4, IDO, KIR, LAG3, NOX2, PD-1, TIM-3, VISTA, SIGLEC7, or combination thereof.

The human HLA class 1 heavy chain sequence, in embodiments, includes HLA-E or a fragment thereof, HLA-F or a fragment thereof, HLA-G or a fragment thereof, or any combination thereof. In embodiments, at least one of the HLA-E or the fragment thereof, HLA-F or the fragment thereof, HLA-G or the fragment thereof, or any combination thereof is inhibited from eliciting a T cell response when the complex interacts with one or more T-cells.

The complex, in embodiments, has a regulatory peptide. The regulatory peptide, in embodiments, is an apoptosis-inducing peptide, for example to act as a “kill switch” for controlling the complex. BioNVs, in embodiments, do not necessitate regulatory peptides in their complexes, whereas hypoimmunogenic cells intended for use as cell therapies use regulatory peptides in their complexes.

The complex, in embodiments, has an epitope configured to allow for detection of the complex. In embodiments, the epitope is or comprises 3,5-dinitrosalicylic acid.

The complex, in embodiments, includes a human B2M amino acid sequence. In embodiments, the complex has one or more linkers is between the peptide sequence and the human B2M sequence, or between the human B2M sequence and the human HLA class 1 heavy chain sequence, or one or more linkes between both.

The complex, in embodiments, has one of more linkers. The one or more linkers, in embodiments, is placed between the peptide and the HLA class I sequence; alternatively, the one or more linkers is between the HLA class I sequence and one or more of a B2M, checkpoint agonist sequence, and/or second HLA sequence. The one or more linkers, in non-limiting embodiments, has a sequence with at least about 70%, 80%, 90%, 95%, or 99% sequence identity to an influenza A virus M1 peptide or a histone M3 peptide. The one or more linkers, in non-limiting embodiments, has a sequence substantially comprised of glycine and/or serine residues, such as Gly-Gly-Ser, Gly-Gly-Gly-Ser, Gly-Gly-Gly-Gly-Ser, Gly-Ser-Ser, and the like.

The complex, in embodiments, includes a fusion construct from N-terminus to C-terminus, a peptide (e.g., to bind the HLA groove), one or more linkers, the human 2M sequence, one or more linkers, and the human HLA class 1 heavy chain sequence. The complex, in embodiments, includes one or more HLAs, where the one or more HLAs are inhibited from eliciting a T cell response when the complex interacts with one or more T cells. In embodiments, the HLA fusion has one or more linkers which adopts a conformation that enables interaction (e.g., does not block binding) with one or more killer-cell immunoglobulin-like receptor (KIR) binding sites on the human HLA class 1 heavy chain sequence.

The one or more HLAs, in embodiments, has one or more mutations that reduce or inhibit the HLAs from eliciting a T cell response when the complex interacts with one or more T cells, including cytotoxic T lymphocytes (e.g., CD4+ and/or CD8+ T cells).

The complex, in embodiments, has one or more proteins or fragments thereof that reduce or inhibit an immune response by the complement system. The one or more proteins or fragments thereof, in embodiments, include CD48, CD59, or a combination thereof.

The immune incompetent HLA, in embodiments, is in the form of a nucleic acid molecule which optionally functions to displace one or more sequences encoding a native HLA gene in the hypoimmunogenic cell. In embodiments, the hypoimmunogenic cell has a genetic element configured to receive one or more sequences for deletion/disruption in the HLA locus. The one or more sequences, in embodiments, encode the human HLA class 1 heavy chain sequence fusion complexes described herein.

### BioNVs for Neoantigen Presentation

In aspects, the present disclosure includes BioNVs which have one or more surface-exposed neoantigens. The neoantigen, in embodiments, is or comprises a protein (or peptide derived therefrom) of one or more proteins in Table 5 and/or Table 6, including variants or mutants thereof. In embodiments, the neoantigen is one or more peptides from Table 1. Without wishing to be bound by theory, the one or more biomarkers stimulate a whole cell expressing a cognate CAR in co-therapeutic applications, while also retaining the CAR-directed killing power of the BioNV.

TABLE 1
Illustrative neoantigen amino acid sequences.

| Gene | Mutation | Wild Type Peptide | Exemplary Mutant Peptide |
|---|---|---|---|
| BRAF | V600E | KIGDFGLATVK [SEQ ID NO: 1] | KIGDFGLATEK [SEQ ID NO: 2] |
| KRAS | G12D | KLVVVGAGGV [SEQ ID NO: 3] | KLVVVGADGV [SEQ ID NO: 4] |
| KRAS | G12V | KLVVVGAGGV [SEQ ID NO: 3] | KLVVVGAVGV [SEQ ID NO: 6] |
| KRAS | G12V | KLVVVGAG [SEQ ID NO: 7] | KLVVVGAV [SEQ ID NO: 8] |
| BRAF | V600E | KIGDFGLATVK [SEQ ID NO: 9] | KIGDFGLATEK [SEQ ID NO: 10] |
| PIK3CA | H1047R | AHHGGWTTKM [SEQ ID NO: 11] | ARHGGWTTKM [SEQ ID NO: 12] |
| PIK3CA | E545K | STRDPLSEITE [SEQ ID NO: 15] | STRDPLSEITK [SEQ ID NO: 16] |
| BRAF | V600E | FGLATVKSRW [SEQ ID NO: 17] | FGLATEKSRW [SEQ ID NO: 18] |
| BRAF | V600E | LATVKSRW [SEQ ID NO: 19] | LATEKSRW [SEQ ID NO: 20] |

In embodiments, BioNVs (and the cell that the BioNV is derived therefrom) express an MHC Class I/II receptor to display the neoantigen. In embodiments, the MHC Class I/II receptor is an immune-incompetent HLA molecule, as described herein. In embodiments, the MHC class I/II receptor displays the neoantigen by a direct binding interaction. In embodiments, the MHC class I/II receptor is fused to the neoantigen (e.g., via a flexible peptide linkage). In embodiments, fusion includes one or more amino acid linker sequences, as described herein.

In embodiments, “expression or increased expression” of a “gene and/or gene product” encompasses increased expression at the DNA level (e.g., stable expression from knock-in of the gene, insertion of a transgene cassette with the gene and an enhancer, internal ribosome entry site (IRES) element, etc.), or a change outside of the gene itself which results in increase of that gene product (e.g., increased transcription factor concentration, a higher activity promoter inserted/replaced upstream of the gene, etc.). In embodiments, expressing or overexpressing the one or more immunoprotective proteins is at the RNA level using one or more of a small regulatory RNA, IRES element, modulating the activating transcription factor concentration in the cell, or a combination thereof.

In embodiments, expressing or increasing expression of the one or more immunoprotective proteins is by introduction of an exogenous genetic element. In embodiments, the introduction of the exogenous genetic element is by stable integration into the cell genome. In embodiments, the stable integration is by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, or a combination thereof. In embodiments, the stable integration is by a viral vector. In embodiments, the introduction of the exogenous genetic element is by transient transfection. In embodiments, expressing or increasing expression of the one or more immunoprotective proteins is by an exogenous promoter and/or enhancer, and/or an endogenous promoter and/or enhancer, or a combination thereof. In embodiments, expressing or increasing expression of the one or more immunoprotective proteins is under the control of a constitutively active promoter.

In embodiments, expressing or increasing expression of the one or more immunoprotective proteins is at the DNA level by one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA. In embodiments, expressing or increasing expression of the one or more immunoprotective proteins is at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA. In embodiments, expressing or overexpressing the one or more immunoprotective proteins is by one or more of a small regulatory RNA, miRNA, IRES element, transcription factor, or a combination thereof.

The expression or overexpression of the one or more immunoprotective proteins is by a knock-in of a genetic element. In embodiments, expression or overexpression of the one or more immunoprotective proteins is by a Cre/Lox recombinase system, transposase-based system, or an endonuclease system, as described herein. In embodiments, “knock-in” of a “genetic element” can refer to engineering a promoter/enhancer element upstream of an existing gene, or inserting a new transgene cassette with a copy of the gene in frame with one or more promoter, enhancer, intron, IRES sequence, or any other element that can be used to increase expression of a gene and/or gene product. In embodiments, expression or overexpression is by an exogenous promoter or enhancer (e.g., such as from a plasmid, or a knock-in of a promoter or newly added cis-acting DNA element). In embodiments, expression or overexpression is by an endogenous promoter or enhancer, such as due to an increased concentration of a transcription factor acting on the endogenous promoter (e.g., without genomic manipulation). In embodiments, expression or overexpression is under the control of a constitutively active promoter (e.g., SV40, CMV, UBC, EF1A, PGK CAGG, etc., for mammalian systems) and/or an inducible promoter (e.g., Tetracycline-controlled, etc.).

In embodiments, expression or overexpression is by stably integrating a gene into the cell. In embodiments, stable integration is by a viral vector. In embodiments, iPSCs (among other cells) are genetically engineered for gene cassette integration (e.g., of a CAR or any other element described herein). In embodiments, gene cassette integration can include both integrative and non-integrative transgene insertion. Non-limiting examples of non-integrative transgene insertion include mRNA, non-integrative lentivirus, and endonuclease-targeted methods. Integrative gene cassette insertion methods include stable retroviral vector insertion and transposase-based integration systems. Stable gene cassette transduction can be achieved, for example, using retroviral vectors which can enable iPSCs to maintain the genetic element encoding the gene throughout differentiation, expansion, and activation. In embodiments, clinical-grade, stable transduction of CAR cassettes into T cells has been achieved for brexucabtagene autoleucel (Tecartus®, Kite Pharma Inc.) and axicabtagene ciloleucel (Yescarta®, Kite Pharma Inc.) using GRV vectors, while tisagenlecleucel (Kymriah®, Novartis International AG) is transduced using a lentiviral vector (Labbe, R. P., et al. “Lentiviral Vectors for T Cell Engineering: Clinical Applications, Bioprocessing and Future Perspectives.” Viruses 13 (2021); 1528. doi: 10.3390/v13081528).

In embodiments, the hypoimmunogenic cell can express the one or more immunoprotective proteins by transient transfection (e.g., electroporation, lipid reagent, etc.). In embodiments, the hypoimmunogenic cell can be genetically modified to contain one or more knock-outs of the above immunogenic proteins and the expression or overexpression of one or more immunoprotective proteins can be controlled by transient expression in the later steps of the manufacturing pipeline.

In embodiments, the hypoimmunogenic cell (and BioNV derived therefrom) is allogeneic. In embodiments, allogeneic refers to the “off-the-shelf” quality of the cells originating from a single source to be used to create clonal cell populations adapted to treating multiple diseases and can be administered to multiple patients, regardless of immunological profile of the subject to be treated.

In embodiments, the hypoimmunogenic cell does not cause an immune reaction in patients to which it or a biological composition derived therefrom is administered. For example, in embodiments, the hypoimmunogenic cell does not result in an inflammatory reaction and/or an immune response upon administration. In embodiments, upon administration to a subject, the hypoimmunogenic cell elicits less than about 50%, about 45%, about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% of an inflammatory or immune response measured as a function of cytokine, chemokine, or immunomodulatory enzyme concentration, such as IL-1, IL-2, IL-3, IL-4, IL-6, IL-7, IL-9, IL-10, IL-11, IL-12, IL-13, IL-14, IL-15, IL-16, IL-17, IL-20, IFN-α/β/γ, TNFα/β, IDO, HLA-G, HGF, PGE2, among others, or any combination thereof, in comparison, e.g. to a cognate whole cell therapy counterpart.

In embodiments, the hypoimmunogenic cell comprises one or more targeting agents. In embodiments, the cell comprises one or more targeting agents. In embodiments, the one or more targeting agents comprises a chimeric antigen receptor (CAR). In embodiments, the CAR is bispecific. In embodiments, the CAR lacks an intracellular portion. In embodiments, the CAR comprises a targeting agent, a transmembrane domain, and an intracellular domain, which comprises a costimulatory domain and/or a signaling domain. In embodiments, the transmembrane domain is derived from CD28, CD3ζ, CD4, CD8α, or ICOS, or a fragment thereof. In embodiments, the intracellular domain comprises an intracellular signaling domain of a CD3ζ-chain and/or one or more co-stimulatory molecules, optionally selected from CD28, 4-1BB, ICOS, CD27, and OX40. In embodiments, the one or more targeting agents comprises an antibody or antibody format. In embodiments, the antibody or antibody format is selected from one or more of a monoclonal antibody, polyclonal antibody, antibody fragment, Fab, Fab′, Fab′-SH, F(ab′)2, Fv, single chain Fv (scFv), V_{NAR}, V_{H}H, afflilin, diabody, nanobody, linear antibody, bispecific antibody, multi-specific antibody, chimeric antibody, humanized antibody, human antibody, and fusion protein comprising the antigen-binding portion of an antibody. In embodiments, the antibody format is a scFv. In embodiments, the one or more targeting agents comprises a viral epitope recognition receptor (VERR) or viral ligand. In embodiments, the one or more targeting agents comprises a ligand for a receptor. In embodiments, the one or more targeting agents comprises a receptor for a ligand.

In embodiments, the hypoimmunogenic cell expresses the one or more targeting agents by a regulatable expression element. In embodiments, regulatable expression element includes a regulatable promoter (such as Tet on/off promoter), or CRISPRa/i regulated systems, among other regulatable expression elements. In embodiments, CAR expression is controlled by a regulatable expression element. Simple overexpression of the construct from a CMV (or other type) promoter can lead to surface densities of the CAR that are too high and thus could cause a number of problems such as ‘hyper activation’ leading to in vitro exhaustion and cell death after activation. Cell death results in loss of the cell line for generating BioNVs. In embodiments, the surface density of CAR protein constructs is regulated. For example, in embodiments, the typical concentration range of CAR protein per microgram of T cells is between 0.20 ng-0.70 ng. Too little expression of the CAR results in poor biomarker targeting. However, the density cannot exceed those of the cellular limit. Too high of a CAR density can result in parent cell exhaustion during the activation process in vitro prior to BioNV derivation and can also result in poor quality BioNVs due to high protein concentration in the plasma membrane. An upper limit allows for increasing the targeting efficiency for low biomarker expression on cancer cells. The density limit for cellular therapeutics is listed, for example, in the published U.S. non-provisional application, US20220040106, which is hereby incorporated by reference.

In embodiments, stable cell integration (safe harbor genetic location) of any genetic element described herein in a cell (e.g., iPSCs) can be controlled by implementing a Tet-regulated CRISPRa+targeted 3× transcription factor targeted gRNA system. The CRISPR activation system for three upstream transcription factors can trigger a signal cascade event that enhances the production of CARs that have replaced endogenous antibody ORFs at designated locus (loci). This system can be ‘tunable’ by including a Tet-regulated promoter, allowing for the ability to vary the concentrations of CARs on the surface of the cell. Next, stable cell replacement of CDRs and heavy and light antibody regions with CAR cassettes can be achieved via Cpf-1 directed homology directed repair (HDR). Finally, the stably integrated CAR cassette can contain flanking gRNA binding sites which allow the scFV (among other antibody formats) or VERR/viral ligand to be repeatedly swapped or altered for rapid and consistent insertion of a desired sequence.

In embodiments, the concentration of the CAR on the surface of the iPSC base cell line, or any downstream differentiated cell (and the resulting BioNVs), can be regulated using a variety of transcription control elements, such as a tetracycline on/off promoter (or similar drug-regulated promoters) to drive the expression of a CRISPR activation/gRNA (CRISPRa) system. The CRISPRa system can then activate the antibody-regulating transcription factors, for example, Drm2, Fr5, and Bxp2, which regulate the expression of an engineered CAR cassette that has been integrated at the site of an antibody locus (where the antibody genes have been replaced). Additionally, a similar transcription control element can be provided to control overexpression of genes (e.g., CD47), drive genes controlling differentiation, etc., at defined manufacturing stages.

In embodiments, regulation points within a cell can be engineered to express specific therapeutically relevant proteins of interest as a stable cell line (e.g., stable integration for constitutive expression).

In embodiments, transcription factors can be activated within a cell by supplying a small molecule, (de)phosphorylation event, or nucleic acid, etc. In embodiments, transcription factors can be transiently expressed by plasmids. In embodiments, transcription factors can be stably expressed from integration (e.g., with constitutively active promoters) to generate a stable cell line with constitutive signaling of signaling pathways related to therapeutic biomolecules. In embodiments, therapeutic biomolecules to express in the hypoimmunogenic cell include cytokines (e.g., for functions such as pro-inflammatory, anti-antigen, recruitment, etc.), perforins, granzymes, chemokines, interferons (IFNα/β/γ), interleukins, alarmins, lymphokines, tumor necrosis factors (TNFs), colony-stimulating factors, bone morphogenic protein (BMP), erythropoietin (EPO), granulocyte-stimulating factor (G-CSF), granulocyte macrophage colony-stimulating factor (GM-CSF), or a combination thereof.

In embodiments, the promoter region or enhancer regions of a gene can be regulated by the overexpression of a specific transcription factor, regulated by microRNAs, tRNAs, activated or inhibited using (g) RNA guided endonucleases with activating or inhibitory domains linked to them (e.g., CRIPSRa/CRISPRi).

In embodiments, a gene of therapeutically relevant interest can be integrated (either stably or transiently) into the cell of interest (based on desirable properties such as membrane proteins that infer barrier crossing). In embodiments, stably integrated genes can be activated by any one of method described herein.

In embodiments, background (unwanted) mRNAs can be silenced with interfering RNAs (e.g., siRNA, RNAi, etc.), to enhance the presence/expression of desired mRNAs to proteins of interest. In embodiments, mRNAs can also be regulated through IRES elements. In embodiments, specific spliced variants can be enhanced by the addition of IRES enhancing and/or repressing biomolecules, to produce a desired therapeutically relevant peptide/protein that can be packaged into a BioNV during post-activated cellular processing.

In embodiments, unwanted genes can be knocked-out to enhance the expression of desired therapeutically relevant genes. Those skilled in the art will appreciate the methods available for transient and stable knock-out of undesired genes and/or entire signaling pathways during the processing for BioNVs.

In embodiments, hypoimmunogenic cells are derived from iPSCs that have been engineered as described herein. In embodiments, iPSCs are reverted from a somatic state using microRNA technology in lieu of small molecule trans-activators. The use of microRNA provides a tighter differentiation system and that results in higher quality iPSCs. Without wishing to be bound by theory, these high quality iPSCs are less prone to expression dampening (of post-engineered proteins, such as CD47) and genetic drift, and possess higher culture splitting qualities/quantities (the cultures can be divided more times than other methods before cellular integrity issues occur).

In embodiments, BioNVs derived from iPSC-derived hypoimmunogenic cells which retain the functionality from the hypoimmunogenic cell, for example and without limitation, the ability to cross the blood-brain barrier, such as is the case of macrophages/monocytes, or tissue-specific factors such as is the case in cardiomyocytes, hepatocytes, etc.

In embodiments, allogeneic iPSCs have their MHC class I and MHC class II complexes disrupted by knocking out critical proteins involved in their expression, for example, B2M which is a serum protein found in association with the MHC class I heavy chain on the surface of nearly all nucleated cells that is involved in the presentation of peptide antigens to the immune system.

In embodiments, once the B2M knock-out (KO), CIITA KO, IL-6 KO, and CD47tg knock-in (KI) is engineered into the iPSC, the TRAC and TRBC genes can be knocked out. In embodiments, only one gene for each is knocked out rather than both of on the separate alleles. In embodiments, the TRAC and TRBC genes can be knocked out as described herein. The purpose of knocking out the TRAC and TRBC genes is to eliminate the T-cell receptors. In embodiments, the modified cell is differentiated to a T-cell subset which lacks T-cell receptors to derive the BioNVs. Genetically modifying the cells to substantially lack TCRs reduces the chances for a competing ligand to the CAR construct that can target non-specifically to alternate tissues. Therefore, in embodiments, the TCR genes are knocked-out as a strategy to reduce off-target effects of the BioNVs. In embodiments, TRAC/TRBC knock-outs decrease the likelihood of CRS, as well as BioNV toxicity, generally.

In embodiments, the modified cell is expanded after engineering; any small scale expansion or large-scale feeder system expansion methods known in the art can be used.

In embodiments, after the B2M KO, CIITA KO, IL-6 KO, CD47tg KI, and an IL-2 promoter-driven green fluorescence protein (GFP) (IL-2p GFP) reporter is constructed, the CAR constructs can be integrated/engineered into the cell. In embodiments, the CAR constructs can be knocked-in to the TRAC/TRBC genes, simultaneously knocking-out the remaining TRAC/TRBC genes, resulting in a cell that is CAR+ and TRAC/TRBC−/−. In embodiments, the CAR construct can be knocked-in to the TRAC/TRBC gene location on both loci simultaneously, resulting in a cell that is CAR+/+ and TRAC/TRBC−/−.

In embodiments, once the B2M KO, CIITA KO, IL-6 KO, CD47tg KI, IL-2p GFP KI, and CAR modified cells (e.g., iPSCs) are engineered, the Immunological Synapse (IS) quality is measured between the CAR recognition domains and the biomarker. In embodiments, the quality of the IS of BioNVs can be directly related to efficacy in whole cell therapies.

In embodiments, the BioNVs, or the hypoimmunogenic cell derived therefrom, comprises a nucleic acid encoding GFP (among other fluorescence proteins). In embodiments, once the B2M KO, CIITA KO, CD47tg KI, IL-6 KO, TRAC/TRBC single KOs are engineered into the iPSC, a GFP molecule is engineered into the modified cell line. In embodiments, this serves as the control cell line. In embodiments, the non-control cell line (the therapeutic cell line) does not have GFP. In embodiments, the nucleic acid encoding GFP is operably linked to a promoter from one or more of IL-2, perforin, granzyme, alarmin, TNF, INF, a combination thereof, and/or any other cell-specific gene or reporter gene. The IL-2 promoter is constitutively activated when lymphocytes are broadly/globally activated from various stimuli. In embodiments, a more focused activation/repression (regulation) is used. In embodiments, the IL-2p GFP reporter gene serves as an indicator for the degree of broad/global activation of the cell (as part of the BioNV derivation process). In embodiments, the GFP signal, coupled with immunoblot analysis of cytokine levels (such as perforins, granzymes, alarmins, TNFs, and INFs) allows efficient regulation of the degree of broad/global activation of a lymphocyte when exposed to activating antigens. In embodiments, GFP is used to compare the degrees of activation between manufacturing lots and ensure consistency for therapeutic development.

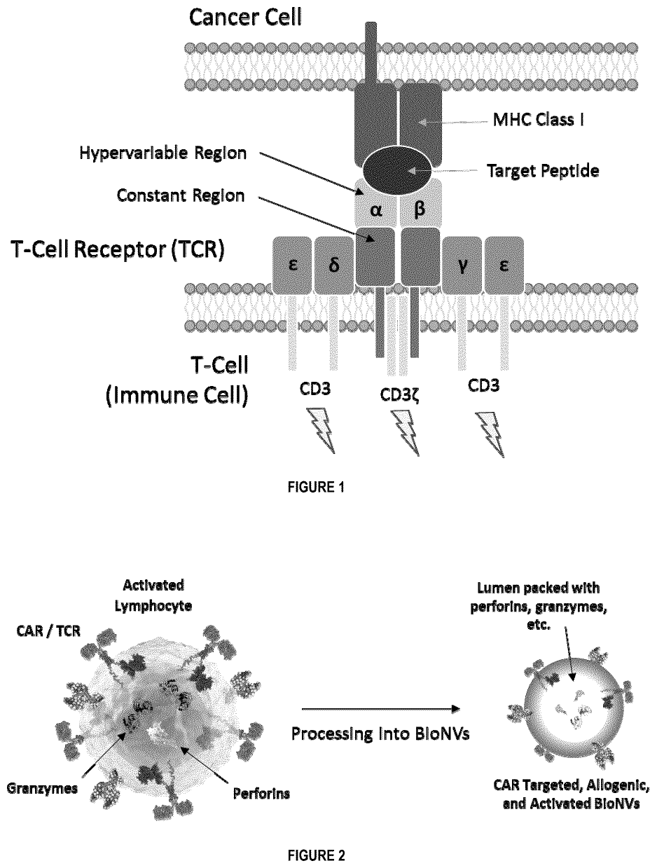

In embodiments, the broad or global regulation of cells results in the expression of multiple genes to produce cytokines, chemokines, regulatory nucleic acids, among other therapeutically-relevant biomolecules that gear a cell into an ‘activated’ state, thereby enhancing the cell to fulfill its metabolically destined purpose/phenotype. In embodiments, controlled expression of the one or more therapeutically-relevant biomolecules in cells can recapitulate, for example, T-lymphocytes entering the activated state that occurs the T cell receptor (TCR) engages an Antigen Presenting Cell (APC) (FIG. 1). In embodiments, controlled expression of the one or more therapeutically-relevant biomolecules in cells can mimic the interaction(s) between the antigen peptide that is presented in the Major Histocompatibility Complex (MHC) and the TCR of the T cell, which initiates conformational changes in the TCR that trigger intercellular signaling cascades and mass gene expression of cytokines including perforins, granzymes, alarmins, interleukins, and interferons to name a few (FIG. 1). Typically, a cytokine ‘activates’ a cell into a specialized mode that allows the cell to clear the antigen from the host and recruit immune cells to the site of infection to aid in the clearance of the antigen/infected cells and repair of the surrounding tissue. In embodiments, these cellular processes occur without supplying an exogenous cytokine, which can lead to deleterious effects in the cell line, such as exhaustion, competing signaling pathways, etc. In embodiments, the activated state of the cell (e.g., T cell) are harnessed in a BioNV/exosome through processing methods in the form of capturing the cytokines or transmembrane ligands in its lumen and/or membrane, as shown in FIG. 2.

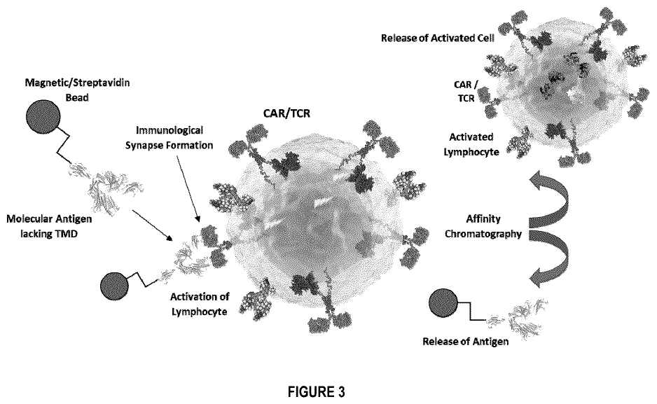

In embodiments, hypoimmunogenic cells are activated and/or purified as shown in FIG. 3. In embodiments, in addition to the pathways of activation, the methods of broad or global cellular regulation includes the conjugation of antigen(s), among other activating molecules, to magnetic or streptavidin-biotin beads to ensure antigen separation from the cell activation receptors (FIG. 3). For example, in embodiments, a biomarker antigen that forms an immunological synapse (IS) with a CAR construct can be conjugated to magnetic or streptavidin-biotin beads and then added to the CAR containing cells in vitro to activate the cells. In embodiments, once activated, the biomarker antigen(s) can be mechanically removed from the cell suspension.

In embodiments, therapeutically relevant biomolecules (e.g., cytokines) can be selectively expressed from within any given cell by manipulating regulation points within points of signaling transduction. In embodiments, one point (or multiple points) within a single signaling pathway (or multiple pathways) can be activated, for example, the activation of a kinase, (de) activation of a phosphorylase, activation of a hydrolase (such as GTPase), introduction of an inhibitor (to diverge or block signals from one pathway to another), or an accelerating activator/agonist. In embodiments, these signaling pathways can be selectively activated by supplying cells with proteins, peptides, small molecules, nucleic acids, carbohydrates, chimeric molecules, viral ligands, inorganic elements/compounds (e.g., calcium), etc., either individually or in combination, to focus cellular pathways to drive expression of therapeutically relevant biomolecules within the cell which can then be packaged into the lumen of a BioNV/exosome (FIG. 3). In embodiments, the points of regulation can include, but are not limited to, one or more cell surface receptors targeted either individually, or in combination, to focus a signal within the cell to express the biomolecules of interest. In embodiments, the cells can be genetically modified so that they are especially sensitive to activation of these pathways resulting in a metabolically/phenotypically tuned cell (of any kind) which BioNVs/exosomes can be made from. In embodiments, cell can be made “especially sensitive” to activation by certain stimuli by overexpression of cell surface receptors, overexpression of intracellular signaling molecules (e.g., STATS, NF-κB, MAPK/ERK/ATM kinases, etc.), and/or constitutively active mutants thereof.

In embodiments, the hypoimmunogenic cells are CD34+, or derived from CD34+ cells, such as human CD34+ cord blood. In embodiments, CD34+ cord blood-derived cell lines may serve as a base cell line for BioNV development, production, and manufacturing for the delivery of gene editing therapeutics. In embodiments, a CD34+ cord blood-derived hypoimmunogenic cell line has been experimentally confirmed for the low expression of HLA 1/2 and overexpression of CD47 (Deuse T. et al. “Hypoimmunogenic derivatives of induced pluripotent stem cells evade immune rejection in fully immunocompetent allogeneic recipients.” Nat Biotechnol. 2019; 37 (3): 252-258).

In embodiments, the hypoimmunogenic cell can be engineered using multiple hypoimmunogenic engineering techniques, for example, as described in Deuse et al., Han et al., Xu et al., Harding et al., and also as described in published U.S. patent applications US20190376045, US20190376045, US20210308183, and US20210292715 to Deuse, US20210161971 to Nagy, US20180141992 to Strominger, and Published European patent application 3693384 to Poirot, each of which is incorporated by reference herein in their entirety (Han X, et al. “Generation of hypoimmunogenic human pluripotent stem cells.” PNAS. Vol. 116, No. 21 2019: pp. 10441-10446. doi: 10.1073/pnas. 1902566116.) and (Xu H, et al. “Targeted Disruption of HLA Genes via CRISPR-Cas9 Generates iPSCs with Enhanced Immune Compatibility.” Cell Stem Cell. Vol. 24, No. 4, 2019: pp. 566-578. doi: 10.1016/j.stem.2019.02.005.).

In embodiments, BioNVs are derived from cells which have eliminated HLA genes that encode the MHC membrane glycoproteins that confer immune reactions associated with GVHD rejections. The HLA gene clusters can be divided into three categories: 1) the MHC Class I pathway, 2) the MHC Class II pathway, and 3) the MHC Class III pathway. Only the MHC Class I and II pathways express the protein complexes elicit an immune response in GVHD, whereas MHC Class III complexes are not involved in immunization activities.

The elimination of the MHC classes of protein complexes can trigger NK cells and macrophages into an active clearance mode where the cells are subsequently destroyed. To avoid this kill mechanism, in embodiments, the addition of a CD47 isoform 2 transmembrane molecular protein tag can be engineered into the cell membrane of the modified cell to avoid NK and macrophage-mediated kill responses, for example, as described in Willingham et al., Deuse et al., and Han et al. (Willingham S B, et al. “The CD47-signal regulatory protein alpha (SIRPa) interaction is a therapeutic target for human solid tumors.” PNAS. Vol. 109, No. 17, 2012: pp. 6662-7. doi: 10.1073/pnas.1121623109.).

In embodiments, cells can be engineered to use additional mechanisms to prevent these kill responses such as those described in: 1) the CD24 transmembrane molecular protein tags (for example as performed in Zhao et al.), 2) the membrane-bound surfactant protein-D (SP-D) (for example as performed in Jiaravuthisan et al.) (Jiaravuthisan P, et al. “A membrane-type surfactant protein D (SP-D) suppresses macrophage-mediated cytotoxicity in swine endothelial cells.” Transpl Immunol. Vol. 47, 2018: pp. 44-48. doi: 10.1016/j.trim.2018.02.003.), and 3) the molecular PD-L1 tag for prevention of T-cell responses. In embodiments, a BioNV derived from an ‘activated’ cell would encapsulate and/or release perforin and/or granzyme, resulting in targeted cell death. In embodiments, the activated cell would generate perforin and/or granzyme to be packaged into the BioNV. In embodiments, hypoimmunogenic cells that are to be activated would not express PD-L1 to avoid the resultant BioNV from being targeted to PD-1 on T-cells. In embodiments, this reduces the likelihood of releasing perforin and/or granzyme, resulting in unwanted T-cell death. In embodiments, PD-L1 is overexpressed in BioNVs derived from a cell that has not been activated and is not loaded with apoptotic cytokines. In embodiments, hypoimmunogenic cells that are to be activated have PD-L1 downregulated, knocked-out, or otherwise silenced. In embodiments hypoimmunogenic cells that are not to be activated have PD-L1 upregulated, i.e. for BioNVs used for gene editor delivery. In embodiments, CD47 isoform 2, can be engineered into the cell to prevent both macrophage and NK cell-mediated cytotoxicity because it acts as a “don't eat me” tag through the SIRP-α receptor that is expressed on these cells, among other cells. In embodiments, CD47 can be utilized in genetically engineered iPSCs for immune tolerance to innate immune cells, for example, such as in Chhabra et al., Han et al., and Jaiswal, et al. (Chhabra A, et al. “Hematopoietic stem cell transplantation in immunocompetent hosts without radiation or chemotherapy.” Sci Transl Med. Vol. 8, No. 351, 2016: 351ra105. doi: 10.1126/scitranslmed.aae0501.) and (Jaiswal S, et al. “CD47 is upregulated on circulating hematopoietic stem cells and leukemia cells to avoid phagocytosis.” Cell. Vol. 138, No. 2, 2009: pp. 271-85. doi: 10.1016/j.cell.2009.05.046.). In embodiments, cells can be modified as described in U.S. Pat. No. 8,562,997 to Jaiswal, et al., which is incorporated by reference herein in its entirety.

In embodiments, some approaches can be used which do not entirely knock-out all HLA genes, for example, as performed in Xu et al. and Han et al., which only knock-out the HLA genes that are highly associated with an immune response, leaving intact the HLA genes that dampen a macrophage or NK response (e.g., HLA-E, HLA-F, and HLA-G). In embodiments, this approach does not require the addition of a CD47 tag; the modified cell can be engineered to generate BioNVs with or without CD47.

In embodiments, methods improve upon the approaches of hypoimmunogenicity of Table 2.

TABLE 2
Three methods of modification of cells using the HLA knockout combined with
a CD47 isoform 2 tag and a PD-L1 transmembrane tag (Zhao, et al.) and
(Gornalusse G G, et al. “HLA-E-expressing pluripotent stem cells escape
allogeneic responses and lysis by NK cells.” Nat Biotechnol. Vol. 35, No. 8,
2017: pp. 765-772. doi: 10.1038/nbt.3860.).

| Source | HLA-A | HLA-B | HLA-C | HLA-E | HLA-F | HLA-G | HLA-II | CD47 | PD-L1 |
|---|---|---|---|---|---|---|---|---|---|
| Gornalusse, | Knockout | | | + | Knockout | | | | |
| 2017 | | | | | | | | | |
| Deuse, | B2M Knockout | | | | | | CIITA | + | |
| 2019 | | | | | | | KO | | |
| Xu, | Knockout | | | | | | CIITA | | |
| 2019 | | | | | | | KO | | |
| Han, | Knockout | | | | | | CIITA | + | + |
| 2019 | | | | | | | KO | | |

In embodiments, developing the allogeneic modified cell involves the removal of MHC Class I and MHC class II protein complexes through the disruption of certain HLA genes, or a B2M knockout, followed by knocking out the CIITA gene. In embodiments, the knockouts can be performed using CRISPR gene editing approaches, due to their rapid mechanism of action. In embodiments, the knockouts are performed using Zinc Finger Nucleases (ZFNs) and/or TALENS. In embodiments, Cre/Lox recombinase systems are used to generate the modified cell. In embodiments, RNA silencing (RNAi, shRNA, microRNA, CRISPR Cas13a-d, etc.) is used to generate the modified cell.

In embodiments, the methods of developing the allogeneic, hypoimmunogenic modified cell is distinct from the methods of creating allogenicity of Harding et al. In lieu of deleting the MHC class I/II genes and running the risk of preventing long-term acceptance by the recipient, the Harding et al. method includes an alternate approach based on immune escape mechanisms that occurs in nature. The method relies on the Harding et al. biomimicry based on the horizontally transmitted cancer, DFTD type 2, that is predominant in Tasmanian devils. In embodiments, developing the allogeneic modified cell include expression or increased expression of the immunomodulatory proteins CCL21, PD-L1, FasL, SerpinB9, H2-M3, CD47, CD200, and/or MFG-E8 to protect cell derivatives from long-term immune rejection in mice (and humans), without the deletion of MHC class I/II proteins. In embodiments, the modified cell expresses one or more of the proteins shown in Table 3, including any splice variant and/or isoform of any of the indicated proteins (e.g., CD200 splice variants). In embodiments, this system can be used to interfere with the activity of APCs, macrophages, NK cells, and T-lymphocytes. In embodiments, the modified cell lines can also contain the safe-cell system developed by Liang et al. 2018, where cell division genes are linked to a suicide gene to prevent runaway teratomas leading to cancers (Liang Q, et al. “Linking a cell-division gene and a suicide gene to define and improve cell therapy safety.” Nature. Vol. 563, No. 7733, 2018: pp. 701-704. doi: 10.1038/s41586-018-0733-7.).

In embodiments, methods improve upon the approaches of hypoimmunogenicity of Table 3.

TABLE 3
Expression or increased expression of illustrative proteins for creating allogenic modified cells.

| Gene | Type of protein | Illustrative Immunomodulatory Function |
|---|---|---|
| Ccl2 | Chemokine | Recruitment of activated DCs |
| PD-L1 | Check Point Inhibitor | Shuts Down Effector T-cell Responses |
| (non-activated | | |
| cells and/or | | |
| BioNVs) | | |
| FASL | Transmembrane Signal | Induces T-cell Apoptosis |
| H2-M3 | Antigen Presentation-MHC-like | Inhibits NK cells |
| SERPINB9 | Inhibitory Chemokine | Blocks granzyme B function |
| | (Serine Protease Inhibitor) | |
| Cd47 | Transmembrane SIRP-αR ligand | Blocks phagocytosis |
| Cd200 | Type 1 Membrane Glycoprotein | Prevents macrophage and granulocyte activation |
| Mfg-e8 | Chemokine (attractant) | Macrophage recruitment to anti-inflammatory profile |
| | | site |

In embodiments, hypoimmunogenic cells from which BioNVs are derived are engineered to have knock-outs of one or more of HLA-A, HLA-B, HLA-C, HLA-E, HLA-G, HLA-F, CIITA, IL-6, IL-4, IL-10, IL-16, TRAC, TRBC, SerpinB9, and/or any combination thereof; and knock-ins of one or more of CCL2, PD-L1 (in BioNVs derived from non-activated cell sources), CTLA-4, H2-M3, CD24, CD47 (minus the 3′ UTR region or an alternate 3′ UTR region that does not contain binding sites for the inhibitory microRNAs), MFG-E8, CD200, and/or any combination thereof.

In embodiments, BioNVs are generated from a hypoimmunogenic cell with one or more of the modifications of Table 4.

TABLE 4
Illustrative engineered cell expression profile for BioNV formation for human use (Fife BT and Bluestone
JA. “Control of peripheral T-cell tolerance and autoimmunity via the CTLA-4 and PD-1 pathways.”
Immunol Rev. Vol. 224, 2008: pp. 166-82. doi: 10.1111/j.1600-065X.2008.00662.x.) and (Rong Z, et al.
“An effective approach to prevent immune rejection of human ESC-derived allografts.”
Cell Stem Cell. Vol. 14, No. 1 2014: pp. 121-30. doi: 10.1016/j.stem.2013.11.014.).

| Knockouts | Knock-ins (inducible or constitutive over-expression) |
|---|---|
| HLA-A | CCL2 |
| HLA-B | PD-L1 (non-activated cells and/or BioNVs) and/or CTLA-4 (Fife et al. |
| | and Rong et al.) |
| HLA-C | H2-M3 |
| HLA-E or HLA-G | CD24 or CD47 or CD47 tethered to CD24 |
| (but not both) | |
| HLA-F | MFG-E8 |
| CIITA | CD200 for non-immunomodulatory functions only (such as liver cell |
| IL-6 | regeneration). These cells will not contain a CD47 tag. |
| IL-4, IL-10, or | SerpinB9 |
| IL-16 (optional) | (where granzyme is not used) |
| TRAC | |
| TRBC | |
| SerpinB9 | |
| (where granzyme is used) | |

In embodiments, inactivation/activation of genes is controlled by inducible promoters throughout the differentiation, activation, and manufacturing process for BioNVs. In embodiments, disruption of MHC, TCR, and CRS genes produce allogeneic iPSCs which are −/− CRS and −/− TCR, leading them to have plasma membranes which exhibit hypoimmunogenic properties upon infusion into a subject. CRS genes implicated in the pathogenesis of CRS include IL-6, IL-10, IFN-γ, monocyte chemoattractant protein 1 (MCP-1), granulocyte-macrophage colony-stimulating factor (GM-CSF), among other cytokines, including tumor necrosis factor (TNF), IL-1, IL-2, IL-2-receptor-a, and IL-8. In embodiments, one or more of these genes is inactivated, e.g., in a cell from which the BioNVs are derived.

In embodiments, BioNVs are formed by disrupting the cell membranes of engineered iPSCs. In embodiments, the hypo-iPSCs are characterized by a B2M−/−, CIITA−/−, CD47+/+, PD1−/− plasma membrane profile and may be used to generate BioNVs. Hypoimmunogenic BioNVs can be generated from the parent iPSC cell line via sonication, adaptive focused acoustics technology, French press, extrusion, serial extrusion, cell lysis by detergent, and electroporation, among other methods. In embodiments, serial extrusion is the method used to generate hypoimmunogenic BioNVs. In embodiments, serial extrusion of iPSCs can produce BioNVs that are HLA1/HLA2 negative (hypoimmunogenic) with tgCD47+, exhibiting PD1 resistance elimination.

BioNVs Generated from Hypoimmunogenic Cells

In aspects, the present disclosure includes BioNVs which are approximately 20-1200 nm in size and, in embodiments, can contain outwardly facing, membrane-embedded targeting agents (e.g., a CAR) capable of binding one or more target molecules. In embodiments, BioNVs are biomimetic due to the nanovesicle composition which originates from the plasma membrane of allogeneic, hypoimmunogenic modified cells. In embodiments, BioNVs comprise plasma membrane-derived lipid bilayers, fully encapsulating an aqueous core which can house a variety of cell-derived molecules, including perforins, granzymes, cytokines, gene editing payloads, etc. In embodiments, the aqueous core of the BioNVs can further enclose exogenous biologics, fluorescent proteins, tracing dyes, radionuclides, and small molecules, among other therapeutic agents, which can be synthesized in the cell before disruption, or added in the cell processing steps.

In embodiments, BioNVs can inherit CAR constructs from the hypoimmunogenic cell, which can comprise a variety of structural molecules. The structure-function of a prototypical CAR includes a fusion protein comprising an extracellular (or outwardly facing) binding moiety (e.g., scFv), connected by a hinge peptide (e.g., CH2/CH3 domains from an IgG Fc region, Gly-Gly-Ser peptide linkage, CD28 peptide, CD8α peptide, etc.) to a transmembrane domain (e.g., CD28, CD3ζ, CD4, CD8α, ICOS, etc.), followed by a variety of intracellular signaling domains (e.g. 4-1BB, CD3ζ, CD28, 4-1BB, ICOS, CD27, OX40, etc.). In embodiments, BioNVs lack the intracellular machinery of whole cells and therefore the CAR design does not necessitate any intracellular signaling molecules (primary CAR construct). In embodiments, the CAR construct includes an extracellular scFV binding moiety fused with an IgG CH2/CH3 linker to a CD28 transmembrane domain and substantially lacks any intracellular domains or functionality.

In embodiments, the CAR constructs have the prototypical intracellular domains swapped or otherwise fused to anchor proteins, e.g., PLA2 domain from an AAV, fusion proteins, radionuclide-binding domains, cytoskeletal elements, small molecule transporting domains, etc., which may aid in the fusion to target cells and/or packaging and release of therapeutic payloads. In embodiments, the BioNV expresses one or more factors that increase uptake, including in non-limiting examples, one or more membrane-embedded proteins, surface-functionalized and/or anionic or cationic conjugated lipids, and/or viral ligand/receptor which improve or facilitate uptake of BioNVs.

In embodiments, CAR antigen-binding molecules comprise a variety of binding moieties, including antibody-based or antibody format binding domains. In embodiments, BioNVs comprise antibody or antibody format binding moieties selected from one or more of a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the CAR construct includes binding moieties with a Bispecific T cell Engager (BiTE), viral epitope recognition receptor (VERR) or viral ligand, variable heavy chain lgG fragment VHH, V_{NAR}, or through an engineered T-Cell Receptor (TCR). In embodiments, BioNVs comprise a ligand for a receptor or a receptor for a ligand as a targeted agent.

To ensure proper directionality of CARs and to eliminate BioNVs lacking CARs, in embodiments, HPLC-based affinity chromatography techniques can be used to select and concentrate only the BioNVs with a sufficient surface concentration of solvent-exposed CARs. HPLC-based affinity chromatography techniques can be used to reduce the concentration of contaminating cell material and NVs which harbor immunogenic cell surface markers, either by positive or negative selection.

In embodiments, BioNVs can include NVs with an outer plasma membrane leaflet only, an inner plasma membrane leaflet only, and/or both leaflets of a plasma membrane lipid bilayer intact. iPSC-derived NVs, in embodiments, include additional lipid additives (e.g., phosphatidylethanolamine, phosphatidylcholine, phosphatidylinositols, ceramides, lecithins, etc.), non-ionic surfactants (e.g., sorbitan monostearate, octadecylamine, etc.), sterols (e.g., cholesterol, bile salt derivatives, etc.), polyols (e.g., maltodextrin, sorbitol, sucrose, mannitol, etc.) and proteins (e.g., serum albumin, Fc, etc.) added for improved physicochemical properties, such as thermal stability, clearance, and therapeutic payload packaging/release. The amount of cholesterol and the length and saturation of the hydrocarbon chains of the phospholipids can affect the rigidity and the stability of the bilayer, and in turn the capability of the NVs to load and release drugs, biomolecules, and other therapeutic payloads. In embodiments, BioNVs also incorporate zwitterionic lipids and methods of using zwitterionic lipids, for example, as described in US Patent Publication No. US20130216607, the contents of which are herein incorporated by reference in its entirety. Correspondingly, functionalization of the hydrophilic heads of the lipids with polymers or biomolecules can provide additional features to the vesicle surface, thus shaping their interaction with blood components, tissues, and the immune system in vivo.

In embodiments, the hypoimmunogenic cell can synthesize one or more therapeutically relevant biomolecules for packaging into a BioNV. In embodiments, the one or more therapeutically relevant biomolecules is a cytokine, pro-inflammatory cytokine, anti-antigen cytokine, perforin, granzyme, chemokine, interferon (IFNα/β/γ), interleukin, alarmin, lymphokine, and tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), or a combination thereof.

In embodiments, the hypoimmunogenic cell can be used to encapsulate a payload; e.g., “lumen-loading”, or the ability of the BioNV that is derived from the cell to have a payload loaded into the lumen (space in the biomimetic nanovesicle). In embodiments, the payload is one or more of a biologic, a nucleic acid, a fusion protein, a fluorescent protein, a tracing dye, a radionuclide, and/or a small molecule. In embodiments, the payload is a therapeutic payload for a disease type that the CAR is targeted against. In embodiments, the payload comprises one or more of an alkylating agent, an anthracycline, an antimetabolite, an anti-tumor antibiotic, an antibody or antibody format, a corticosteroid, a plant alkaloid, a topoisomerase inhibitor, a checkpoint inhibitor, an anti-infective agent, and/or a growth factor.

In embodiments, the nucleic acid payload encodes one or more of a CRISPR/Cas component, guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, ribosomal RNA (rRNA), short hairpin (shRNA) complementary messenger RNA, repeat associated small interfering RNA (rasiRNA), and/or small non-coding RNA.

In embodiments, the payload includes gene editing nucleic acids and/or proteins, such as for example, TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog of any of these editors. In embodiments, the gene editors can also include gRNA, which, as used herein, refers to guide RNA. In embodiments, the gRNA can be a sequence complimentary to a coding or a non-coding sequence and can be tailored to the particular sequence to be targeted. In embodiments, the gRNA can be a sequence complimentary to a protein coding sequence, for example, a sequence encoding one or more viral structural proteins, (e.g., gag, pol, env and tat). In embodiments, the gRNA sequence can be a sense or anti-sense sequence. In embodiments, when a gene editor composition is administered herein, preferably without limitation, including two or more gRNAs; however, a single gRNA can also be used.

In embodiments, BioNVs deliver a gene editing payload comprising a transactivating response region (TAR) loop system. In embodiments, the BioNV encapsulates a plasmid which expresses a gene editor and contains a TAR loop sequence between the 5′ end of the promoter and the gene editor/guide cassette and acts as a barrier, blocking transcription. In embodiments, transcription will only trigger in cells that are infected and contain the HIV Tat protein. In embodiments, the Tat protein binds to the TAR Loop, relaxes it, and frees the promoter for transcription, thereby expressing the editor and its guides.

In embodiments, primary targeted BioNVs are used to deliver small molecule therapeutic payloads. In embodiments, second generation (or 3rd or 4th gen) CAR-containing BioNVs derived from activated lymphocytes can contain cytokines and other cytotoxic peptides. In embodiments, BioNVs can be formatted to encapsulate and deliver plasmid DNA, for example, to express gene editing nucleases and gRNA in target cells. Alternatively or additionally, in embodiments, BioNVs can encapsulate the nucleases and gRNA. In embodiments, targeted second generation (or 3rd or 4th gen) BioNVs can be designed to encapsulate and deliver additional therapeutic proteins or peptides of interest.

In embodiments, the BioNV comprises one or more of CD34, CCL21, PD-L1 (non-activated cell source) and/or CTLA-4, FasL, SerpinB9, H2-M3; one of either CD24, CD47, CD200, chimeric CD24/CD47, chimeric CD24/CD200, and chimeric CD47/CD200; or two of either CD24, CD47, and CD200; MFG-E8, NCAM, α-phagocytic integrin, anti-IL-6R antibody or antibody format, and/or fusions or portions thereof. In embodiments, BioNV can have NCAM or any other protein that facilitates fusion of the BioNV to a plasma membrane of a target cell. In embodiments, BioNVs that encapsulate at least one granzyme can lack SerpinB9 and/or CD200. In embodiments, BioNVs that express PD-L1 are derived from non-activated cells and the BioNVs substantially lack perforin and/or granzyme.

In embodiments, the BioNV substantially lacks one or more of MHC class I complexes, MHC class II complexes, HLA-A complexes, HLA-B complexes, HLA-C complexes, HLA-E or HLA-G complexes, HLA-F complexes, T cell receptor alpha chain (TRAC) proteins, T cell receptor beta chain (TRBC) proteins, PD-1, SerpinB9, IL-4, IL-6, IL-10, and/or IL-16. In embodiments, the BioNV substantially lacks one or more of the above to be allogeneic and/or hypoimmunogenic. In embodiments, the BioNV does not produce an adverse immune reaction upon infusion into a subject for treating a disease.

In embodiments, the BioNV comprises one or more targeting agents. In embodiments, the cell from which the BioNV is derived can be modified to express the one or more targeting agents. In embodiments, the one or more targeting agents is a CAR. In embodiments, the one or more targeting agents can be any antibody or antibody format described herein.

In embodiments, the BioNVs are targeted against one or more biomarkers of Table 5 and/or Table 6.

TABLE 5
Illustrative checkpoint biomarkers and ligands of checkpoint biomarkers.

| Biomarker | Illustrative Function |
|---|---|
| CTLA-4 | Immune checkpoint |
| PD-L1 | Suppress adaptive immune system |
| PD-L2 | Immune checkpoint receptor ligand |
| B7-H3 | Immune checkpoint |
| B7-H4 | Immune co-inhibitory molecule |
| BTLA | Immune regulatory receptor |
| HVEM | Receptor binding TNF receptor associated factor |
| | molecules |
| TIM3 | Immune response regulator |
| GAL9 | Induces T-cell death |
| LAG3 | Immune checkpoint |
| VISTA | Immune regulatory receptor |
| KIR | Regulate immune tolerance |
| 2B4 | Regulate NK cells |
| CD160 | Checkpoint inhibitor |
| CGEN-15049 | Immune checkpoint |
| CHK1 | Initiate DNA damage response |
| CHK2 | Regulates cell division |
| A2aR | Signals adenosine |
| B-7 family ligands | Regulate immune response |

TABLE 6
Illustrative cancer cell biomarkers.

| Biomarker | Illustrative Function |
|---|---|
| AFP (alpha-feroprotein) | Hepatocellular carcinoma (HCC) |
| Mesothelin | mesothelioma |
| ER (estrogen receptor) | Breast cancer |
| PR (progesterone receptor) | Breast cancer |
| HER-2/neu | Breast cancer |
| EGFR (epidermal | non-small-cell lung cancer |
| growth factor receptor) | |
| KRAS | non-small-cell lung cancer, colorectal cancer |
| UGT1A1 | colorectal cancer |
| c-KIT | Blood, gastric, skin cancers |
| CD30 | lymphomas |
| PDGFR (platelet derived | Gastrointestinal cancer |
| growth factor receptor) | |
| TEM8 (tumor endothelial | lung, breast, and colorectal cancer |
| marker 8) | |
| EIIIB | Various solid tumors |
| CA-125 | Ovarian cancer |
| CD2 | Adhesion between T cells and other cell types and T-cell activation |
| CD9 | Renal cell carcinoma |
| CD11b | Various tumors |
| CD18 | Various tumors |
| CD19 | lymphomas |
| CD20 | lymphomas |
| CD21 | lymphomas |
| CD22 | endometrial cancer, intestinal cancer, skin cancer, leukemia |
| CD24 | Regulation of B-cell proliferation and differentiation |
| CD30 | lymphomas |
| CD31 | Angiosarcoma, Cell adhesion. Plays a key role in leukocyte trafficking |
| | across endothelium |
| CD32a | Dormant T cells harboring viruses |
| CD38 | Cell adhesion and signal transduction |
| CD41 | Ovarian cancer |
| CD42b | lymphomas |
| CD43 | Breast, colon, gastric, lung cancers |
| CD44 | Cell adhesion and migration |
| CD48 | Cell adhesion and T-cell costimulation |
| CD51 | Colorectal cancer |
| CD54 | Colorectal cancer, Cell adhesion, lymphocyte activation, and migration |
| CD61 | Breast, ovarian cancer |
| CD62P | Platelet activation |
| CD63 | Lung cancer |
| CD66a | Neutrophil activation, homophilic and heterophilic adhesion |
| CD66b | Cell adhesion and neutrophil activation |
| CD66c | Cell adhesion and neutrophil activation |
| CD66e | Homophilic and heterophilic adhesion. May play a role in the metastasis |
| | of cancer cells |
| CD74 | MHC class II antigen processing |
| CD87 | Cell chemotaxis and adhesion |
| CD96 | Involved in adhesive interactions of activated T cells and NK cells |
| CD98 | Cell activation and cell aggregation |
| CD105 | Angiogenesis. Regulatory component of TGF-beta receptor complex. |
| | Modulates cellular response to TGF beta 1 |
| CD106 | Leukocyte adhesion. Transmigration and costimulation of T cells. |
| CD107a | Degranulation marker |
| CD111 | Entry mediator for several herpes viruses (HSV1, HSV2, PRV, BHV1). |
| | Believed to be involved in cell adhesion mechanisms |
| CD112 | Intracellular adhesion, component of adherins junctions. Receptor for |
| | herpes simplex virus |
| CD117 | Crucial for development of gonadal and pigment stem cells |
| CD126 | Receptor for IL-6 |
| CD130 | Transmits activities of IL-6, IL-11, LIF and CNF |
| CD138 | Cell proliferation, cell migration, and cell-matrix interactions |
| CD140b | Cell proliferation, differentiation and survival |
| CD143 | Converts angiotensin I to angiotensin II by release of terminal His-Leu, |
| | which results in an increase of the vasoconstrictor activity of . |
| | angiotensin |
| | Cell adhesion. |
| CD144 | Cell adhesion. |
| CD146 | Cell adhesion. Involved in heterophilic cell to cell interactions |
| CD147 | Cell adhesion and T-cell activation |
| CD166 | Mediates cell adhesion by binding to CD6 for intrathymic T-cell |
| | development |
| CD171 | Multidomain cell adhesion molecule required for normal |
| | neurohistogenesis |
| CD202b | Signaling, migration and adhesion |
| CD208 | Dendritic cell maturation marker, involved in cell proliferation |
| CD221 | Receptor for IGF-I and IGF-II. Mediates mitogenic and anti-apoptotic |
| | signals |
| CD227 | Cell adhesion and signaling |
| CD239 | Laminin alpha-5 receptor |
| CD309 | Activation of this receptor by VEGF plays a role in angiogenesis, |
| | development, wound healing, cell proliferation, migration and |
| | differentiation |
| CD321 | Plays a role in epithelial tight junction formation, regulation of |
| | leukocyte erythrocytes. migration, epithelial barrier maintenance and |
| | platelet activation |
| CD324 | Calcium-dependent cell adhesion protein |
| CD326 | A homotypic calcium-independent T-cell adhesion molecule |
| CD333 | Cell-surface receptor for fibroblast growth factors |
| CD340 | Binds tightly to other ligand-bound EGF receptor family members to |
| | form a heterodimer, stabilizing ligand binding and enhancing kinase- |
| | mediated activation of downstream signaling pathways |
| CLEC-2 | Hepatocellular and gastric cancers |
| CD147 | Bladder cancer |
| GPVI | Contributes to thrombosis in cancer |

In embodiments, the BioNV is formed by disrupting the activated cell by one or more of sonication, adaptive focused acoustics technology, French press, extrusion, serial extrusion, enzymatic rupture of cells (e.g., trypsinization), cell lysis by detergent, and/or electroporation. In embodiments, the cell disruption is by serial extrusion.

In embodiments, BioNVs can be analyzed for homogeneity of size by dynamic light scattering (DLS), flow cytometry, mass photometry, among other methods of determining particle size. In embodiments, BioNVs can be filtered for a particle size, or range of sizes, to optimize renal clearance and other clinically-relevant NV properties. In embodiments, BioNVs are about 20 nm to 1200 nm in size. In embodiments, BioNVs can be about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. In embodiments, BioNVs range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size, about 200 nm to 400 nm in size, about 400 nm to 600 nm in size, about 600 nm to 800 nm in size, about 800 nm to 1000 nm in size, or about 1000 nm to 1200 nm in size.

### Methods of Treating Mammalian Diseases

In embodiments, hypoimmunogenic cells can be used to treat mammalian diseases. In embodiments, the BioNVs derived from the hypoimmunogenic cells can be used to treat mammalian diseases. In embodiments, the mammalian disease is a cancer, infectious disease, hereditary disorder, and/or orphan disease.

In embodiments, methods of treating mammalian diseases herein include co-administering a second whole cell therapy (e.g., T cell, NK cell, TIL, macrophage therapy). In embodiments, supplementing a whole cell therapy with the hypoimmunogenic cell and/or BioNV can be used to decrease the effective dosage of the whole cell therapy needed, reducing CRS, off-target effects, teratoma potential, and the like.

In embodiments, methods of treating mammalian diseases include administering an additional therapeutic agent. In embodiments, the additional therapeutic agent can be any additional anti-cancer agent, anti-infective agent, analgesic, and/or non-steroidal inflammatory agent (NSAID).

In embodiments, the hypoimmunogenic cell can be frozen at about −80 °C or is suitable for storage at −80 °C. In embodiments, the BioNVs can be frozen at about −80 °C or is suitable for storage at −80 °C and/or lyophilized (e.g., for reconstitution in buffer). In embodiments, the hypoimmunogenic cell and/or BioNV can be stable at about ambient temperature, at about −20 °C, at about 4 °C, at about 25 °C, or at about 37 °C for at least about 1 hour, at least about 2 hours, at least about 4 hours, at least about 6 hours, at least about 12 hours, at least about 24 hours, at least about 2 days, at least about one week, or at least about one month or longer.

In embodiments, treating mammalian disease can be achieved within about 2 weeks, within about 4 weeks, within about 6 weeks, within about 12 weeks, within about 18 weeks, within about 24 weeks, within about 6 months, within about 1 year, or within about 2 or more years from administration of the composition and methods with such compositions.

### Dosing and Administration

The dosage of any hypoimmunogenic cell (or BioNV) disclosed herein as well as the dosing schedule can depend on various parameters and factors, including, but not limited to, the specific hypoimmunogenic cell, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the disease being treated, the severity of the disorder, and the anatomical location of the disorder. Some variations in the dosage can be expected.

In embodiments, delivery of a BioNV can be like that of a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

Methods of treating mammalian diseases using hypoimmunogenic cell described herein, in embodiments, include dosage ranges in concentration of number of hypoimmunogenic cell per kilogram (kg) subject body weight. In embodiments, suitable dosage ranges for methods described herein can include from about 10³ cells/kg (or BioNVs/kg) to about 10⁹ cells/kg (or BioNVs/kg). In embodiments, the hypoimmunogenic cells are present in the composition at a concentration of about 10³ cells/mL (or BioNVs/mL) to about 10⁹ cells/mL (or BioNVs/mL). Alternatively, in embodiments, hypoimmunogenic cell (or BioNV) compositions are present in compositions as weight/volume in the range of about 5 ng/ml to about 500 mg/mL. In embodiments, the BioNV dosages are based on the size of the BioNV used for the treatment, for example, BioNVs at 1000 nm are provided in approximately 5-fold to 10-fold fewer amounts than 100 nm BioNVs for a comparable dose.

In embodiments, BioNVs (or hypoimmunogenic cells) disclosed herein are administered by a controlled-release or a sustained-release means or by delivery of a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled or sustained-release of one or more active ingredients using, for example, hydropropylmethyl cellulose, other polymer matrices, gels, permeable membranes, osmotic systems, multilayer coatings, microparticles, microspheres, or a combination thereof, to provide the desired release profile in varying proportions. Controlled-or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials are used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In embodiments, a controlled-release system is placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249: 1527-1533 may be used.

In embodiments, the methods using hypoimmunogenic cells (or BioNVs) include applying hypoimmunogenic cell to a surface of a device (e.g., a catheter) or contained within a pump, patch, or other drug delivery device. The excipient or carrier can be selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, the hypoimmunogenic cell (or BioNV) can be administered at doses that are congruent to dosages of whole cells, for example, based on CAR concentration. In embodiments, the typical concentration range of CAR protein per microgram of T cells is between 0.20 ng-0.70 ng, whereas a single BioNV may have a total number of CARs that is 5 times to 10,000 times less than the whole cell, resulting in a conversion of BioNV mass to CAR concentration, where the CAR concentration can be assumed equivalent (such as the case in exosomes) or increased (such as the case in BioNVs) to the cell from which it originated (e.g., the T cell). In embodiments, the concentration and/or surface density of the targeting agent (e.g., CAR) is increased on the BioNV compared to the whole cell from which is it derived. In embodiments, the concentration and/or surface density of the targeting agent (e.g., CAR) is enriched by serial extrusion processing of the whole cell. In embodiments, the concentration and/or surface density of the targeting agent (e.g., CAR), among other cell surface molecules, on the BioNV is 2-fold to 100-fold increased relative to the whole cell. In embodiments, due to exosomes being naturally shed, their concentration and/or surface density of the targeting agent (e.g., CAR), among other cell surface molecules, is substantially the same as the whole cell.

The dosage regimen utilizing any hypoimmunogenic cell (or BioNV) disclosed herein can be selected in accordance with a variety of factors including cancer type, species, age, weight, sex, and medical condition of the subject; the severity of the condition to be treated; the route of administration; the renal or hepatic function of the subject; the pharmacogenomic makeup of the individual; and the specific composition of the disclosure employed. Any hypoimmunogenic cell (or BioNV) disclosed herein can be administered in a single daily dose, or the total daily dosage can be administered in divided doses of two, three, or four times daily. Furthermore, any hypoimmunogenic cell (or BioNV) disclosed herein can be administered continuously rather than intermittently throughout the dosage regimen.

In embodiments, hypoimmunogenic cells (or BioNVs) are administered in consecutive doses about every hour, about every 2 hours, about every 6 hours, about every 12 hours, about every 24 hours, about every 2 days, about every 4 days, about every 7 days, about every 2 weeks, about every 4 weeks, or about every month.

### Additional Therapeutic Agents

In embodiments, the compositions or methods described herein further comprise a therapeutically effective amount of one or more additional therapeutic agents. In embodiments, the therapeutically effective amount of one or more additional therapeutic agents may be in solution with a BioNV, adsorbed onto the surface of the NV, or a payload encapsulated within a BioNV. In embodiments, the additional therapeutic agent is one or more of a checkpoint inhibitor, an analgesic, and/or an anti-infective agent.

In embodiments, the present compositions or methods contemplate other additional therapeutic agents, for example, an analgesic, to aid in treating inflammation or pain at the site of the administration, or an anti-infective agent to prevent infection of the site of treatment with the composition. Non-limiting examples of additional therapeutic agents include analgesics, such as nonsteroidal anti-inflammatory drugs, opiate agonists and salicylates; anti-infective agents, such as anthelmintics, antianaerobics, antibiotics, aminoglycoside antibiotics, antifungal antibiotics, cephalosporin antibiotics, macrolide antibiotics, miscellaneous B-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens; immunobiological agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents; minerals; vitamins, such as water soluble or fat soluble vitamins, vitamin A, vitamin B, vitamin C, vitamin D, vitamin E, and/or vitamin K; and radionuclides such as Yttrium-90, lodine-131, Samarium-153, Lutetium-177, Astatine-211, Lead-212/bismuth-212, Radium-223, Actinium-225, and Thorium-227.

Additional non-limiting examples of useful therapeutic agents from the above categories include: (1) analgesics in general, such as lidocaine or derivatives thereof, and NSAID analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) Hi-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous B-lactam antibiotic anti-infectives, such as aztreonam and imipenem; (10) penicillin antibiotic anti-infectives, such a s nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, remdesivir, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H2-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B12) and niacin (vitamin B3); (38) vitamin C compounds, such as ascorbic acid; and (39) vitamin D compounds, such as calcitriol.

Compositions of Hypoimmunogenic Cells and/or BioNVs

In aspects, the present disclosure relates to compositions that can be used for the treatment of mammalian diseases comprising an allogeneic, hypoimmunogenic cell and/or BioNV. In embodiments, the hypoimmunogenic cell and/or BioNV comprising one or more therapeutically relevant biomolecules.

In embodiments, compositions include hypoimmunogenic cells. In embodiments, compositions can include BioNVs. In embodiments, compositions include hypoimmunogenic cells and BioNVs. In embodiments, compositions include at least one or more of an anti-cancer therapeutic, anti-infective therapeutic, or gene editing payload. In embodiments, composition include a hypoimmunogenic cell (or BioNV) which can adsorb therapeutic molecules onto the surface of and/or encapsulate a therapeutic payload. In embodiments, the composition comprises a therapeutically effective amount of the hypoimmunogenic cells and/or BioNVs.

In embodiments, the composition is allogeneic and/or hypoimmunogenic. In embodiments, the composition is derived from iPSCs (among other cell types) which have been modified to reduce expression of immunogenic molecules and/or increase expression of immunoprotective molecules.

In embodiments, the composition of the hypoimmunogenic cell and/or BioNV is hypoimmunogenic. For example, in embodiments, the composition does not result in an inflammatory reaction and/or an immune response upon administration. In embodiments, upon administration to a subject, the composition, optionally the hypoimmunogenic cells therein, elicits less than about 50%, about 45%, about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% of an inflammatory or immune response measured as a function of cytokine, chemokine, or immunomodulatory enzyme concentration, such as IL-1, IL-2, IL-3, IL-4, IL-6, IL-7, IL-9, IL-10, IL-11, IL-12, IL-13, IL-14, IL-15, IL-16, IL-17, IL-20, IFN-α/β/γ, TNFα/β, IDO, HLA-G, HGF, PGE2, among others, or any combination thereof, in comparison, e.g. to a cognate whole cell therapy counterpart.

In embodiments, the hypoimmunogenic cells are present in the composition at a concentration of about 10³ cells/mL (or BioNVs/mL) to about 10⁹ cells/mL (or BioNVs/mL). Alternatively, in embodiments, cells and/or BioNV compositions are present in compositions as weight/volume in the range of about 5 ng/ml to about 500 mg/mL.

In embodiments, the composition is substantially free of one or more bacteria, virus, fungus, spore, mycoplasma, pyrogen, and in more particular embodiments, is substantially free of all the foregoing. In embodiments, the BioNV composition is substantially free of whole cells and intracellular cell components including organelles such as nuclei, mitochondria, Golgi, etc., and/or substantially free of non-CAR-expressing NVs and/or substantially free of ruptured, damaged NVs. In embodiments, the composition is substantially free of extracellular chromatin, nucleosomes, and other genetic material and non-therapeutic nucleic acids. In embodiments, BioNV compositions are substantial free of cellular genomic DNA.

In embodiments, hypoimmunogenic cells are modular and allogeneic (off-the-shelf) due to the lack of immunogenicity from engineered iPSCs. In embodiments, the lack of whole cell signaling components in BioNVs allows them to be easily tunable for target specificity and resistance to immunosuppressive signals. In embodiments, hypoimmunogenic cells lack the genetic elements that contribute to runaway cytokine storms, minimizing patient risk of CRS. In embodiments, the amounts of active cytokine, perforin, granzymes, interferon, interleukins, etc., encapsulated within the BioNV is regulated during upstream (pre-BioNV derivation) cellular processes. In embodiments, hypoimmunogenic cells are derived from cells capable of crossing biological barriers and/or viral receptors known for facilitation crossing.

Without wishing to be bound by theory, hypoimmunogenic cells generated from iPSC engineered allogeneic base cell lines represent immune invisible cells, meaning that hypoimmunogenic cell has the potential for multi-dosing, and that antibody-mediated neutralization is minimized, and immune cell-mediated clearance is evaded (T cell and macrophage). In embodiments, BioNVs derived from hypoimmunogenic cells do not contain viable genetic material from the cells they were derived to cause CRS or teratoma. In embodiments, increased expression of certain cytokines in the hypoimmunogenic cell is encapsulated within a BioNV which can recruit natural T cells. In embodiments, BioNVs can be derived from modified cell types with or without barrier penetrating ligands to further control activity post-infusion.

Pharmaceutical Compositions and Formulations of Hypoimmunogenic Cells and/or BioNVs

In aspects, the composition is a pharmaceutical composition. In embodiments, the pharmaceutical compositions of the present disclosure are formulated to provide a therapeutically effective amount of hypoimmunogenic cell as the active ingredient. In embodiments, the pharmaceutical compositions of the present disclosure are formulated to provide a therapeutically effective amount of one or more anticancer therapeutics as a payload within a BioNV as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea, and the like. In addition, auxiliary, stabilizing, thickening, lubricating, and coloring agents can be used. Pharmaceutically acceptable excipients are generally sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Any composition disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the composition comprises an excipient or carrier. In embodiments, the diluent can be a pharmaceutically acceptable excipient or carrier.

In embodiments, the pharmaceutical composition comprises a pharmaceutically acceptable diluent. Non-limiting example of diluents include liquid diluents such as water, ethanol, propylene glycol, glycerin, and various combinations thereof, and inert solid diluents such as calcium carbonate, calcium phosphate or kaolin. In embodiments, the diluent comprises one or more of saline, phosphate buffered saline, Dulbecco's Modified Eagle Medium (DMEM), alpha modified Minimal Essential Medium (alpha MEM), Roswell Park Memorial Institute Media 1640 (RPMI Media 1640), HBSS, human albumin, Ringer's solution, and the like, or any combination thereof.

In embodiments, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. In embodiments, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. In embodiments, the resulting compositions can include additional agents, such as preservatives, cryopreservatives (e.g., DMSO), and/or lyoprotectants (e.g., polyols, salts). In embodiments, the carrier can be, or can include a lipid-based or polymer-based colloid. In embodiments, the carrier material can be a colloid formulated as a liposome, a hydrogel, a microparticle, a nanoparticle, or a block copolymer micelle. In embodiments, the carrier material can form a capsule, and that material may be a polymer-based colloid.

In embodiments, the pharmaceutical compositions comprising the hypoimmunogenic cell include a solubilizing agent. In embodiments, the pharmaceutical compositions comprising the hypoimmunogenic cell include a cryoprotective agent or an agent to improve thermal stability, such as DMSO or glycerol. The pharmaceutical compositions, in embodiments, can be delivered with a suitable vehicle or delivery device as known in the art.

In embodiments, the composition comprises a scaffold. In embodiments, the scaffold comprises biomaterials. In a non-limiting example, the three-dimensional biomaterials include hypoimmunogenic cell embedded in an extracellular matrix attached to, or dispersed within, or trapped within the scaffold. In embodiments, the biomaterials are biodegradable and/or synthetic.

In embodiments, the scaffold comprises biodegradable biomaterials. Non-limiting examples of biodegradable biomaterials include fibrin, collagen, elastin, gelatin, vitronectin, fibronectin, laminin, reconstituted basement membrane matrix, starch, dextran, alginate, hyaluron, chitin, chitosan, agarose, sugars, hyaluronic acid, poly (lactic acid), poly(glycolic acid), polyethylene glycol, decellularized tissue, self-assembling peptides, polypeptides, glycosaminoglycans, derivatives and mixtures thereof. Other useful biodegradable polymers or polymer species include, but are not limited to, polydioxanone, polycarbonate, polyoxalate, poly (α-ester), polyanhydride, polyacetate, polycaprolactone, poly(ortho Esters), polyamino acids, polyamides, and mixtures and copolymers thereof, L-lactic acid and D-lactic acid stereopolymers, copolymers of bis(para-carboxyphenoxy) propanoic acid and sebacic acid, sebacic acid copolymers, caprolactone Copolymer, poly (lactic acid)/poly (glycolic acid)/polyethylene glycol copolymer, polyurethane and poly(lactic acid) copolymer, polyurethane and poly(lactic acid) copolymer, α-amino acid copolymer, α-amino acid and caproic acid copolymer, A-benzylglutamate and polyethylene glycol copolymers, succinate and poly(glycol) copolymers, polyphosphazenes, polyhydroxy-alkanoates and mixtures thereof. Binary and ternary systems are also contemplated. In embodiments, the scaffold comprises one or more of collagen, various proteoglycans, alginate-based substrates, and chitosan. In embodiments, the scaffold comprises one or more of a hydrogel, silk, Matrigel, acellular and/or decellarized scaffolds, poly-s-caprolactone scaffolds, resorbable scaffolds, and nanofiber-hydrogel composite.

In embodiments, the scaffold comprises synthetic biomaterials. Non-limiting examples of synthetic biomaterials include lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters.

In embodiments, the compositions can be prepared in any manner well known in the pharmaceutical arts, and can be administered by a variety of routes (e.g., subcutaneous, intravenous, etc.) depending upon whether local or systemic treatment is desired and upon the area to be treated. In embodiments, administration can be topical (including ophthalmic and to mucous membranes including intranasal, vaginal, and rectal delivery), pulmonary (e.g., by inhalation or insufflation of powders or aerosols, including by nebulizer; intratracheal, intranasal, epidermal and transdermal), ocular, oral, or parenteral. In embodiments, methods can include ocular delivery, topical administration (eye drops), subconjunctival, periocular or intravitreal injection or introduction by balloon catheter or ophthalmic inserts surgically placed in the conjunctival sac. In embodiments, parenteral administration includes intravenous, intra-arterial, subcutaneous, intraperitoneal or intramuscular injection or infusion; or intracranial, e.g., intrathecal or intraventricular administration. In embodiments, parenteral administration can be in the form of a single bolus dose, or may be, for example, by a continuous perfusion pump.

In embodiments, pharmaceutical compositions and formulations for topical administration may include transdermal patches, ointments, lotions, creams, gels, drops, suppositories, sprays, liquids, powders, and the like. In embodiments, methods of treating and/or preventing cancer include the use of pharmaceutical carriers, aqueous, powder or oily bases, thickeners, and the like.

In embodiments, the pharmaceutical compositions contain, as the active ingredient, nucleic acids and vectors described herein in combination with one or more pharmaceutically acceptable carriers. In embodiments, the terms “pharmaceutically acceptable” (or “pharmacologically acceptable”) refer to molecular entities and compositions that do not produce an adverse, allergic or other untoward reaction, when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. In embodiments, the term “pharmaceutically acceptable carrier,” includes any and all solvents, dispersion media, coatings, antibacterial, isotonic and absorption delaying agents, buffers, excipients, binders, lubricants, gels, surfactants and the like, that may be used as media for a pharmaceutically acceptable substance.

In embodiments, the compositions can be applied to a surface of a device (e.g., a catheter) or contained within a pump, patch, or other drug delivery device. In embodiments, the compositions can be administered alone, or in a mixture, in the presence of a pharmaceutically acceptable excipient or carrier (e.g., physiological saline). The excipient or carrier is selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, the compositions, e.g., pharmaceutical compositions, disclosed herein are suspended in a saline buffer (including, without limitation, TBS, PBS, and the like).

The present technology includes the disclosed hypoimmunogenic cell in various formulations of pharmaceutical compositions. Hypoimmunogenic cell (or BioNVs) disclosed herein, in embodiments, can take the form of solutions, suspensions, emulsion, drops, tablets, pills, pellets, capsules, capsules containing liquids, powders, sustained-release formulations, emulsions, aerosols, sprays, suspensions, or any other form suitable for use.

Pharmaceutical compositions comprising the hypoimmunogenic cell described herein may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any hypoimmunogenic cell disclosed herein are formulated in accordance with routine procedures as a pharmaceutical composition adapted for a mode of administration disclosed herein.

Subjects and/or Animals

In embodiments, the subject and/or animal intended for use with hypoimmunogenic cells and/or BioNVs is a mammal, e.g., a human, mouse, rat, guinea pig, dog, cat, horse, cow, pig, rabbit, sheep, or non-human primate. In embodiments, the subject and/or animal is a non-mammal, for example, a zebrafish. In embodiments, the subject and/or animal is a transgenic animal comprising a fluorescent cell, such as, for example, an RPE cell and/or an immune cell with GFP. In embodiments, the subject and/or animal is a human. In embodiments, the hypoimmunogenic cells originate from fluorescently tagged cells and/or are packaged with fluorescently-tagged proteins or tags (with e.g., GFP). In embodiments, the human is a pediatric human, human adult, geriatric human, an infant or child. In other embodiments, the human is referred to as a patient or subject.

In embodiments, the method of treatment includes administering to a human who has an age in a range of from about 0 months to about 6 months old, from about 6 months to about 12 months old, from about 12 months to about 18 months old, from about 18 months to about 36 months old, from about 1 year to about 5 years old, from about 5 years to about 10 years old, from about 10 years to about 15 years old, from about 15 years to about 20 years old, from about 20 years to about 25 years old, from about 25 years to about 30 years old, from about 30 years to about 35 years old, from about 35 years to about 40 years old, from about 40 years to about 45 years old, from about 45 years to about 50 years old, from about 50 years to about 55 years old, from about 55 years to about 60 years old, from about 60 years to about 65 years old, from about 65 years to about 70 years old, from about 70 years to about 75 years old, from about 75 years to about 80 years old, from about 80 years to about 85 years old, from about 85 years to about 90 years old, from about 90 years to about 95 years old or from about 95 years to about 100 years old.

In embodiments, the subject is a non-human animal, and therefore the disclosure pertains to veterinary use. In embodiments, the non-human animal is a household pet. In embodiments, the non-human animal is a livestock animal.

In embodiments, sera and/or immune cells and/or tumor cells are evaluated and/or effected. In embodiments, immune cells include cells of a subject's and/or animal's innate immune system. In embodiments, such cells include, but are not limited to NK cell, monocyte, DC, B cell, macrophage, CD4+ T cell, and CD8+ T cell. In various embodiments, the disclosure provides for detecting a presence, detecting an absence, or measuring an amount of tumor volume, tumor cells, metastasis, cDNA, or RNA in a sample originating from a subject.

### Kits

The disclosure, in embodiments, provides kits that can simplify the administration of any agent described herein. An exemplary kit of the disclosure comprises any agent/composition described herein in unit dosage form. In embodiments, the unit dosage form is a container, such as a pre-filled syringe, which can be sterile, containing any agent described herein and a pharmaceutically acceptable carrier, diluent, excipient, or vehicle. In embodiments, the kit further comprises a label or printed instructions instructing the use of any agent described herein. In embodiments, the kit also includes a lid speculum, topical anesthetic, and a cleaning agent for the injection surface. In embodiments, the kit further comprises one or more additional agents described herein.

In aspects, the present disclosure includes a syringe comprising one or more compositions of the present disclosure. In embodiments, the syringe is prefilled with a volume of the composition. In embodiments, the syringe is prefilled in a volume of about 1 mL to about 10 mL. In embodiments, the syringe is prefilled in a volume of about 10 mL, about 9 mL, about 8 mL, about 7 mL, about 6 mL, about 5 mL, about 4 mL, about 3 mL, about 2 mL, about 1.9 mL, about 1.8 mL, about 1.7 mL, about 1.6 mL, about 1.5 mL, about 1.4 mL, about 1.3 mL, about 1.2 mL, about 1.1 mL, or about 1.0 mL or less of the composition.

In embodiments, the syringe comprises a composition having a shelf stability ranging from about 1 hour to about 1 week. In embodiments, the syringe comprises a composition having a shelf stability of at least about 12 hours, about 24 hours, about 36 hours, about 48 hours, or about 72 hours when stored at a temperature ranging from about −85 °C to about 25 °C. In embodiments, the syringe comprises a composition having a shelf stability of at least about 12 hours, about 24 hours, about 36 hours, about 48 hours, or about 72 hours when stored at a temperature ranging from about 15 °C to about 25 °C.

In embodiments, the storage temperature is about −80 °C. In embodiments, the storage temperature is about −20 °C. In embodiments, the storage temperature is about 4 °C. In embodiments, the storage temperature is about 21 °C. In embodiments, the kit includes lyophilized BioNVs.

In one embodiment, the kit comprises a container containing a composition comprising CDV/exosomes of the present disclosure, a therapeutically effective amount of an additional therapeutic agent, such those described herein, and instructions for use.

### EXAMPLES

### Example 1: Constructing a Hypoimmunogenic Cell Line for Generating BioNVs

Hypoimmunogenic cells will be made from human fibroblast induced pluripotent stem cells (iPSCs). Genetic amendments to human fibroblast iPSCs will be done using lentiviral transduction with a CRISPR Cas9 system. Design of each gRNA with minimal off-targets effects for each target loci will be engineered. Lentiviral approaches are suitable for generating BioNVs as the results product is not a cell-based therapy.

The following knock-outs (KOs) will be implemented sequentially:

### Option 1:

HLA-A, HLA-B, HLA-C, HLA-E/G (choose the gene that corresponds with the best fit gRNAs by design for CRISPR Cas9), and HLA-F;

### Option 2:

HLA A, HLA B, HLA C, HLA E/G, HLA F with each HLA knocked-out simultaneously (or in two steps) using current CRISPR Cas9 shotgun-based approaches. Knocking-out the HLA's simultaneously to ensure permanent silencing of the B2M loci will have no lethal or phenotypic effect (outside the expression of MHC I);

### MHC Class II NO:

CIITA; and

### CRS-Related Cytokines:

IL-6, IL-4, IL-10, and/or IL-16. Cytokines will be sequentially knocked-out, starting with IL-6. CRS effect will be evaluated with the IL-6 KO, and more cytokines will be eliminated if the IL-6 KO resulted in CRS or CRS-related phenotypes.

The following knock-ins (KIs) will be implemented sequentially:
CCL2, CTLA-4, H2-M3, MFG-E8;
CD24, CD200, and/or CD47 anti-phagocytic transmembrane domains; and
IL-2p GFP construct (as a reporter for diagnostics and preclinical experiments). Single integration with drug/reporter selection markers will be used for each KI.

Table 7 below details the steps with which cell line banks and the engineering order will be performed.

TABLE 7
Cell line banking and the order of engineering.

| Step No. | Cell Line Genetic Amendment(s) |
|---|---|
| 1 | HLA A, HLA B, HLA C, HLA E/G, HLA F Double KO for each |
| 2 | HLA A, HLA B, HLA C, HLA E/G, HLA F Double KO for each, CIITA Double KO |
| 3 | HLA A, HLA B, HLA C, HLA E/G, HLA F Double KO for each, CIITA Double KO, |
| | CCL2 KI |
| 4 | HLA A, HLA B, HLA C, HLA E/G, HLA F Double KO for each, CIITA Double KO, |
| | CCL2 KI, CTLA-4 KI |
| 5 | HLA A, HLA B, HLA C, HLA E/G, HLA F Double KO for each, CIITA Double KO, |
| | CCL2 KI, CTLA-4 KI, H2-M3 KI |
| 6 | HLA A, HLA B, HLA C, HLA E/G, HLA F Double KO for each, CIITA Double KO, |
| | CCL2 KI, CTLA-4 KI ,H2-M3 KI, CD24 KI |
| 7 | HLA A, HLA B, HLA C, HLA E/G, HLA F Double KO for each, CIITA Double KO, |
| | CCL2 KI, CTLA-4 KI, H2-M3 KI, CD24 KI, MFG-E8 KI |
| 8 | HLA A, HLA B, HLA C, HLA E/G, HLA F Double KO for each, CIITA Double KO, |
| | CCL2 KI, CTLA-4 KI, H2-M3 KI, CD24 KI, MFG-E8 KI, IL-2p GFP KI |
| 9 | After 7 and 8, the CAR cassettes will be designed and integrated |
| 10 | IL-6 Double KOs after the CAR cassettes are integrated |

KO = knock-out; KI = knock-in.

All cell lines will be tested during engineering for Mycoplasma, HBV, HIV, and other standardized contaminant screening. Clonal evaluation of cell line suitability for gene editing will be performed between genetic amendments, including the suitability of the types of growth media, conditions such as splitting time, cell viability, etc.

CRISPR and/or TUNR (AMSBIO) design, assembly, and validation will first be determination to evaluate if any of the HLA and/or CIITA gene KOs are lethal. TUNR is a product that integrates a segment of DNA into the gene of interest to reduce its expression to levels just above lethality, if necessary for cell line development.

Transfection/nucleofection and transduction with lentiviral systems to be used for knock-in and knockout engineering respectively. The KO's and KIs will be conducted in pools at each step, followed by single cell dilution, cloning, drug selection/genetic testing, and maintenance of single cell derived clones. The clones at each step will be expanded for the subsequent steps.

Single cell derived clonal pools will be screened by deep sequencing to prepare a genetic library to enable deep sequencing runs and analyses for additional clonal analyses. Data will be collected and provided at each step. For KOs using CRISPR, the cells with the lowest indel incidence will be selected for further steps in the engineering process.

Final positive clonal expansion (after KOs and KIs, prior to CAR platform intersecting line) will be performed to undergo final genotype confirmation, clonal cryopreservation.

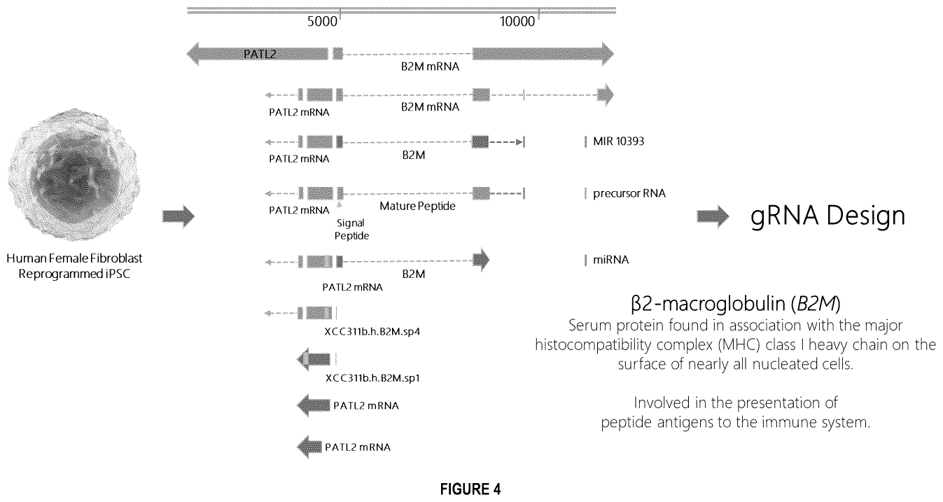

Examples of genetic amendments for generating the human fibroblast reprogrammed iPSC cell lines are illustrated in FIGS. 4-26, which collectively illustrate CRISPR/Cas9 gRNA design, knock-outs efficacy, and off-target analyses, for sequential B2M and CIITA KO, human CD47 (hCD47) isoform 2 knock-in, TRAC and TRBC1 KO in human fibroblast reprogrammed iPSCs. Collectively, clonal sequencing data demonstrates that each of B2M, CIITA, TRAC, and TRBC1 (one of the TRBC genes) were successfully targeted by the gRNA, and that CD47 was successfully knocked-in as evidenced by RNA extraction.

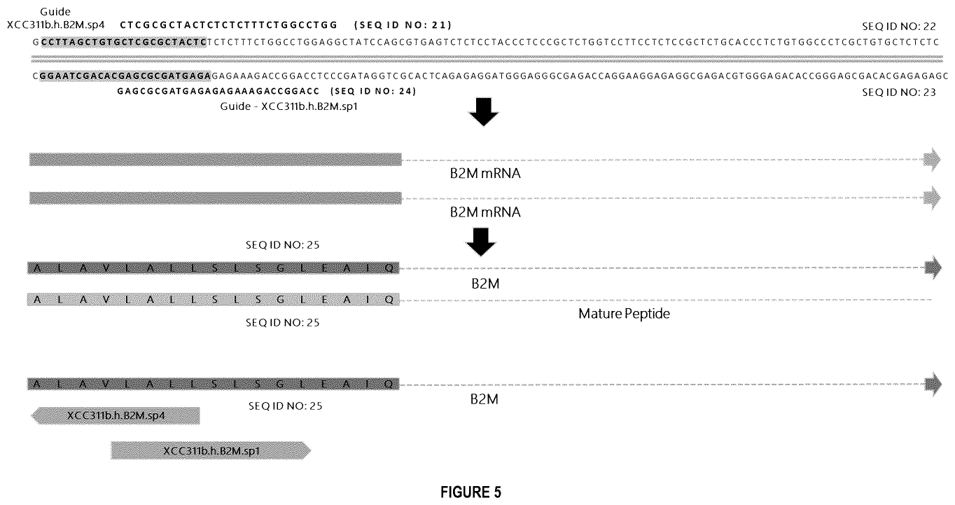

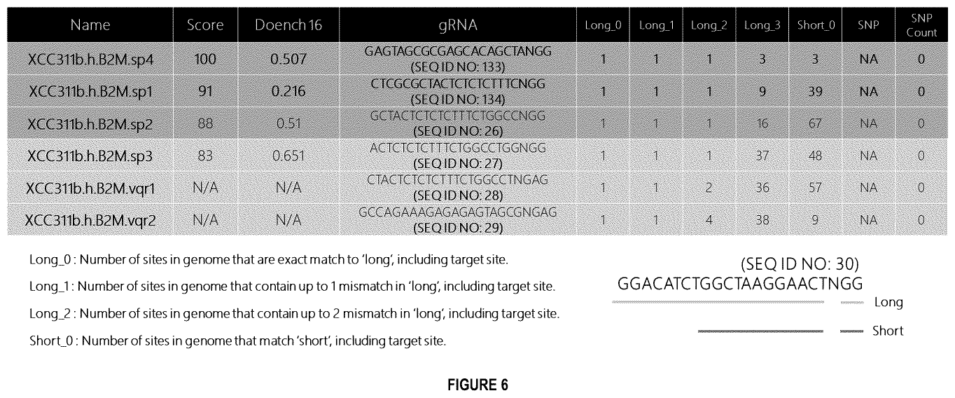

A strategy for B2M knockout is illustrated, e.g., in FIG. 4, which depicts a graphical representation of a B2M genetic knock-out strategy for hypoimmunogenic cell line development from human fibroblast reprogrammed iPSCs. B2-macroglobulin (B2M) is a serum protein found in association with the major histocompatibility complex (MHC) class I heavy chain on the surface of nearly all nucleated cells involved in the presentation of peptide antigens to the immune system. FIG. 5 depicts a graphical representation of a B2M gRNA design for hypoimmunogenic cell line development. The strategy, in embodiments, involves using CRISPR/Cas9 and a specifically designed gRNA to target relevant regions of the B2M gene (e.g., SEQ ID NOs: 22-23), for example gRNAs of SEQ ID NOs: 21, 24, 26-29, and 133-134. FIG. 6 depicts a tabular representation of a B2M gRNA off-target analysis for hypoimmunogenic cell line development.

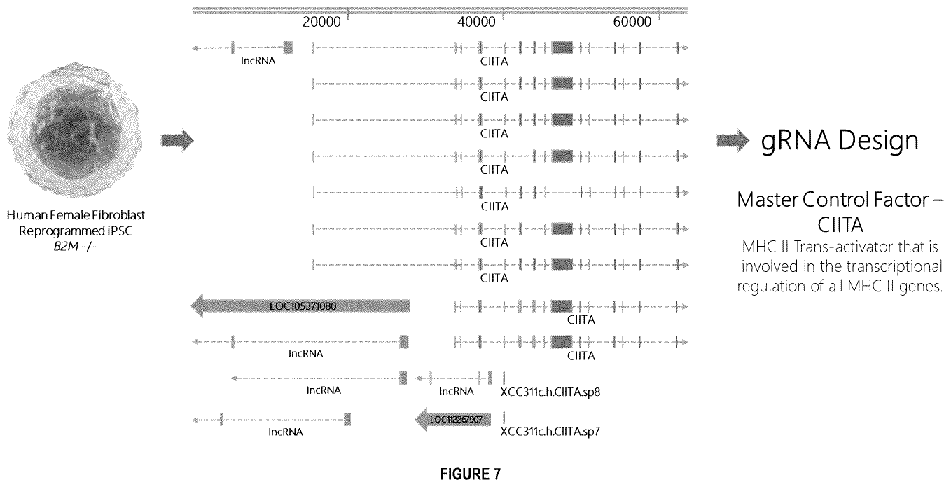

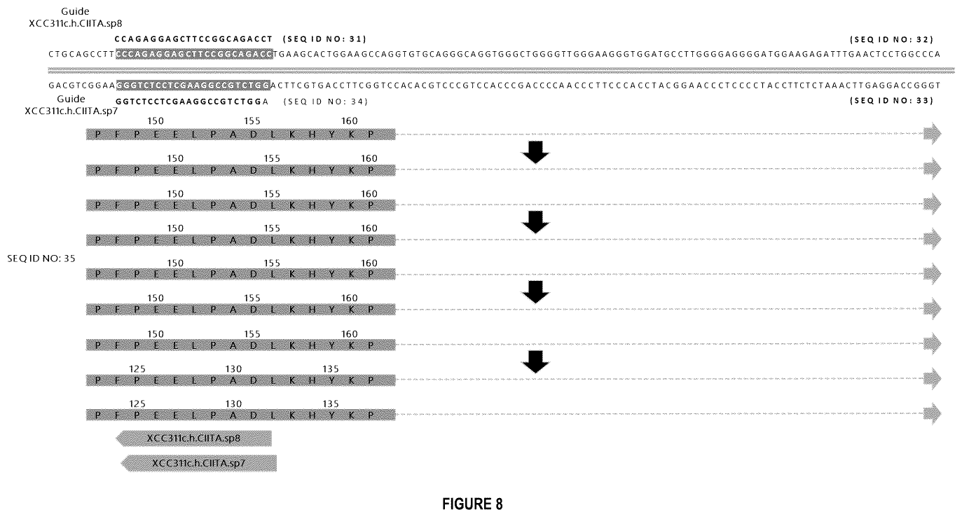

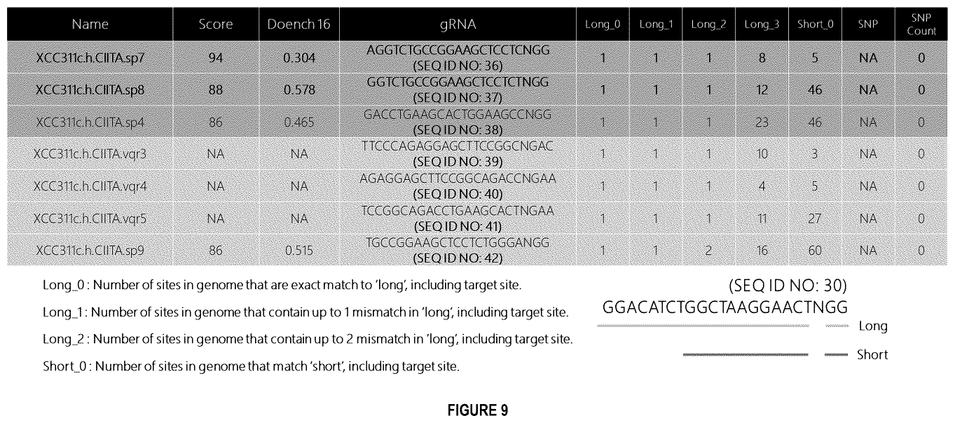

A strategy for CIITA knockout is illustrated, e.g., in FIG. 7, which depicts a graphical representation of a B2M genetic knock-out strategy for hypoimmunogenic cell line development from human fibroblast reprogrammed iPSCs. Master Control Factor CIITA is an MHC class II Trans-activator that is involved in the transcriptional regulation of all MHC II genes. FIG. 8 depicts a graphical representation of a CIITA gRNA design for hypoimmunogenic cell line development. The strategy, in embodiments, involves using CRISPR/Cas9 and a specifically designed gRNA to target relevant regions of the CIITA gene (e.g., SEQ ID NOs: 32-33), for example gRNAs of SEQ ID NOs: 31, 34, and 36-42. FIG. 9 depicts a tabular representation of a CIITA gRNA off-target analysis for hypoimmunogenic cell line development.

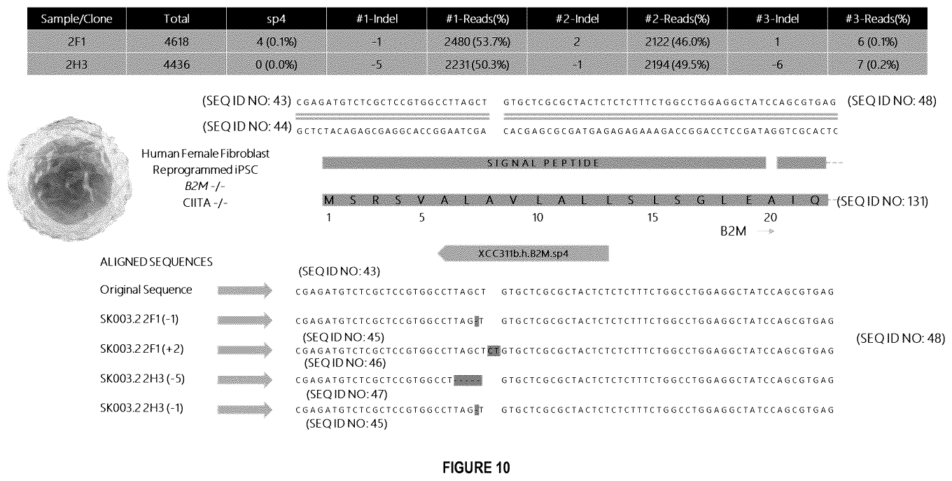

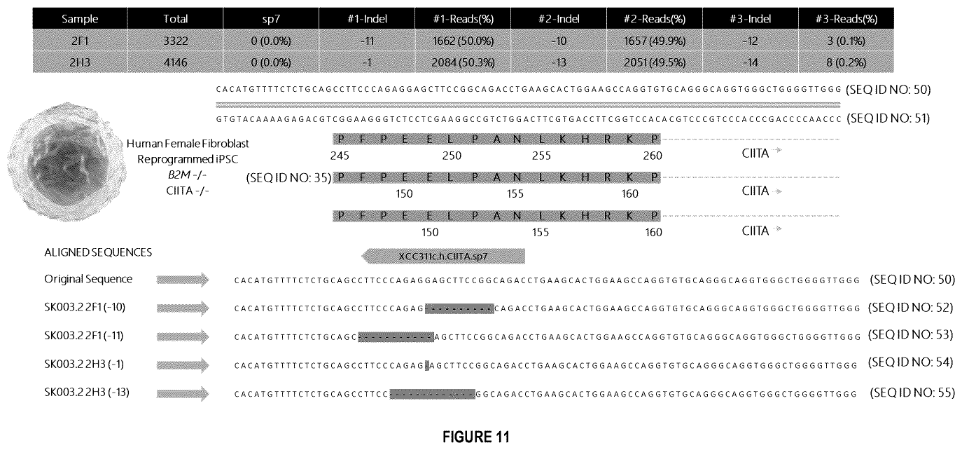

FIG. 10 depicts a graphical representation of a B2M knock-out clonal sequence analysis for hypoimmunogenic cell line development from human fibroblast reprogrammed iPSCs. The results for two selected B2M/CIITA double knock-out clones are shown. Sequencing data illustrate successful B2M KO in the double KO clones, with resultant genomic sequences of SEQ ID NOs: 43-44. FIG. 11 depicts a graphical representation of a CIITA knock-out clonal sequence analysis for hypoimmunogenic cell line development from human fibroblast reprogrammed iPSCs. The results for two selected B2M/CIITA double knock-out clones are shown. Sequencing data illustrate successful CIITA KO in the double KO clones, with resultant genomic sequences of SEQ ID NOs: 50-55.

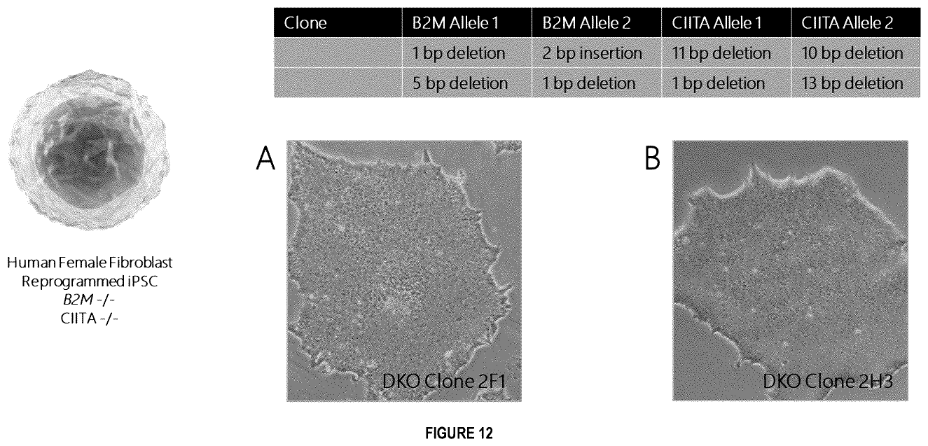

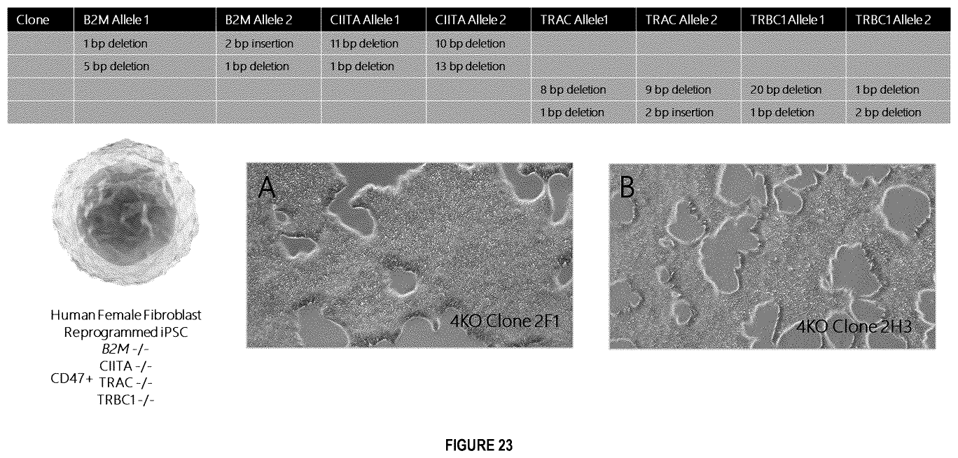

Morphological analyses by light microscopy (e.g., FIGS. 12 and 23) demonstrate that the clonal cell populations appear to have normal morphology and are capable of replication. FIG. 12 depicts a graphical representation of the B2M/CIITA double knock-out clonal sequence summary and clonal morphology in vitro human fibroblast reprogrammed iPSCs hypoimmunogenic cells. The data illustrate that the clonal populations resulted in different overall knock-outs. FIG. 23 depicts a graphical representation of the B2M/CIITA double knock-out, TRAC/TRBC1 double knock-out, hCD47 KI human fibroblast reprogrammed iPSCs hypoimmunogenic cells. The data illustrate the clonal in vitro morphology analysis.

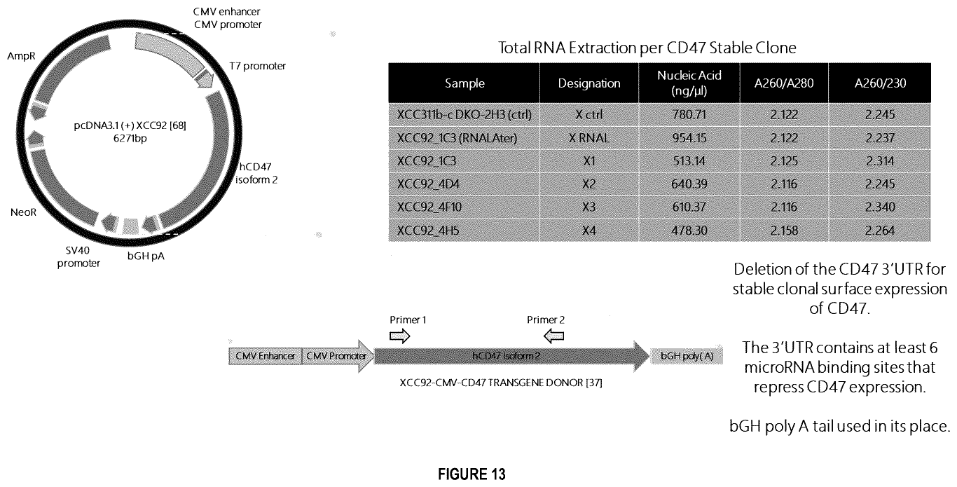

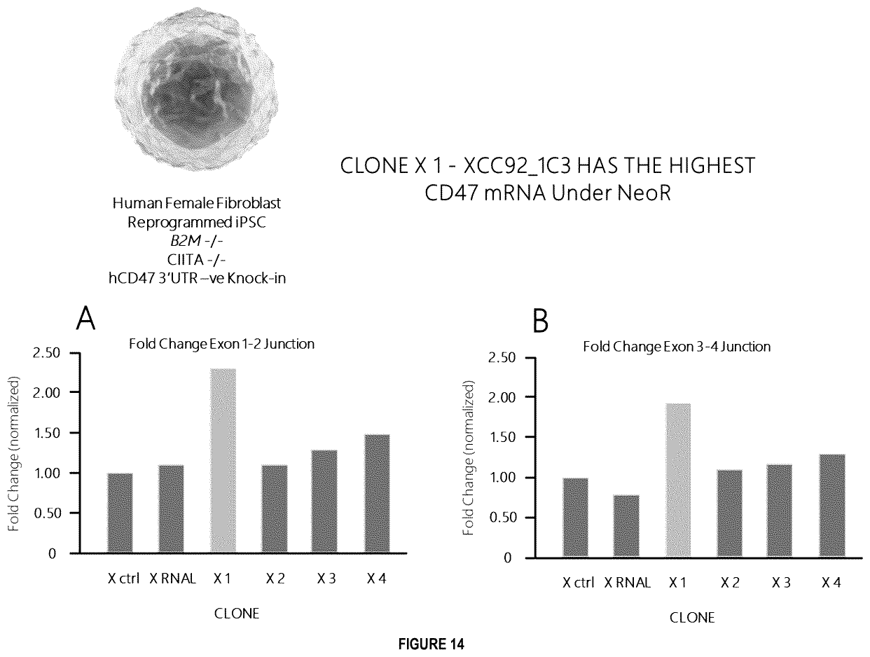

FIG. 13 depicts a graphical representation of a human CD47 (hCD47) isoform 2 knock-in for hypoimmunogenic cell line development from human fibroblast reprogrammed iPSCs. A pcDNA3.1 (+) XCC92 mammalian vector (6271 bp length) is used with selection markers. Deletion of the CD47 3′UTR is performed for stable clonal surface expression of CD47. The 3′UTR contains at least 6 microRNA binding sites that repress CD47 expression, where a bGH poly A tail used in its place. FIG. 14 depicts a graphical representation of hCD47 isoform 2 knock-in clonal selection for hypoimmunogenic cell line development in B2M/CIITA double KO human fibroblast reprogrammed iPSCs. Data indicate successful hCD47 KI in the double KO cell line. The data demonstrate that approximately a two-fold increase in hCD47 at exon 1-2 and 3-4 junctions.

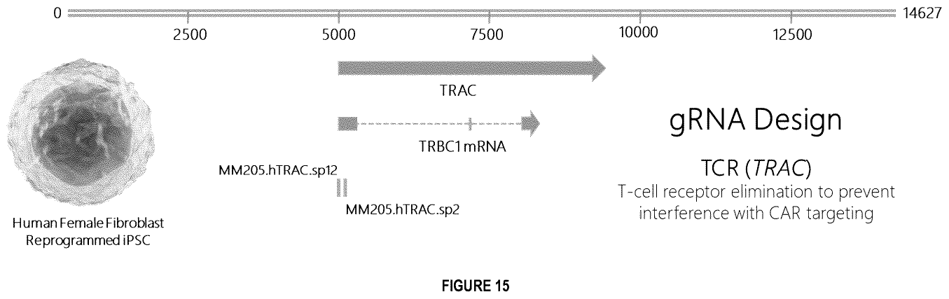

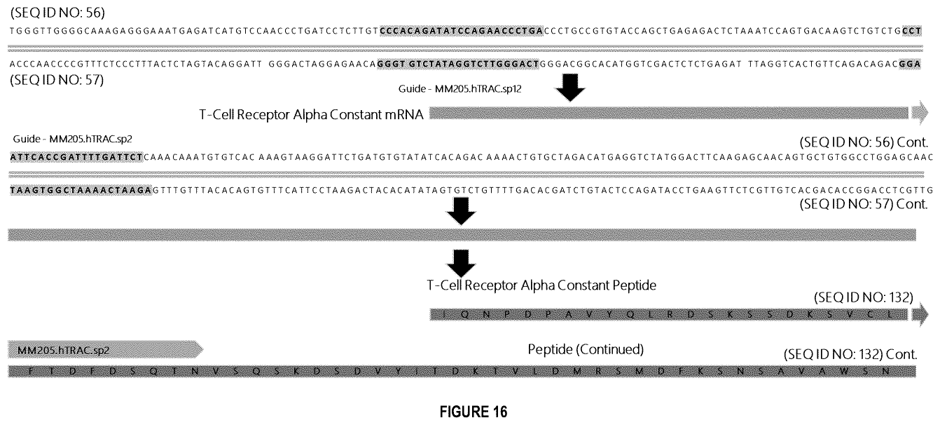

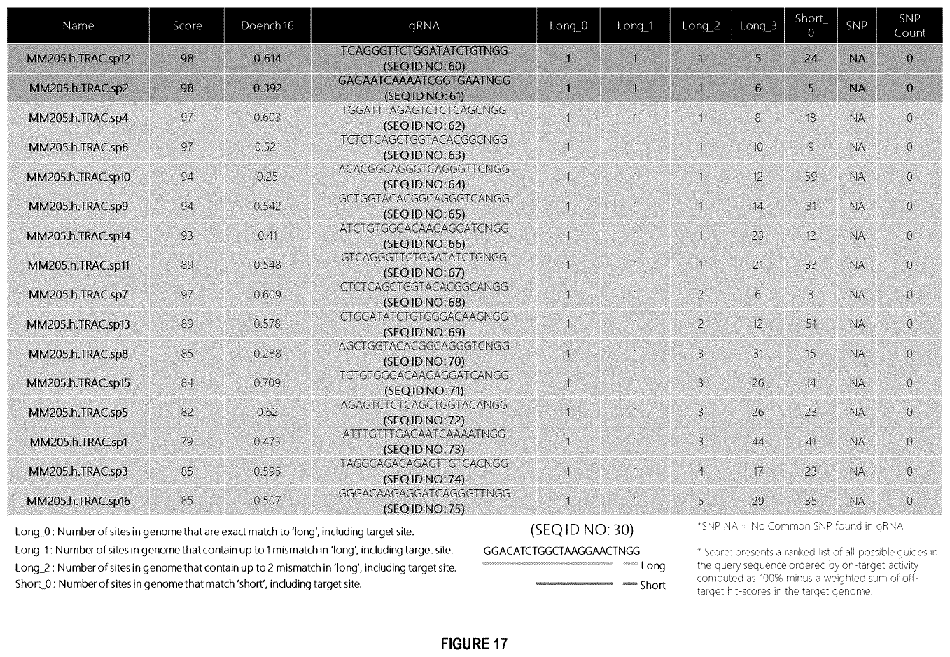

FIG. 15 depicts a graphical representation of a TRAC genetic knock-out strategy for hypoimmunogenic cell line development from human fibroblast reprogrammed iPSCs. In embodiments, the T cell receptor alpha chain (TRAC) elimination is done to prevent interference with CAR targeting and off-target effects. FIG. 16 depicts a graphical representation of a TRAC gRNA design for hypoimmunogenic cell line development. The strategy, in embodiments, involves using CRISPR/Cas9 and a specifically designed gRNA to target relevant regions of the TRAC gene (e.g., SEQ ID NOs: 56-57), for example gRNAs of SEQ ID NOs: 60-75. FIG. 17 depicts a tabular representation of a TRAC gRNA off-target analysis for hypoimmunogenic cell line development.

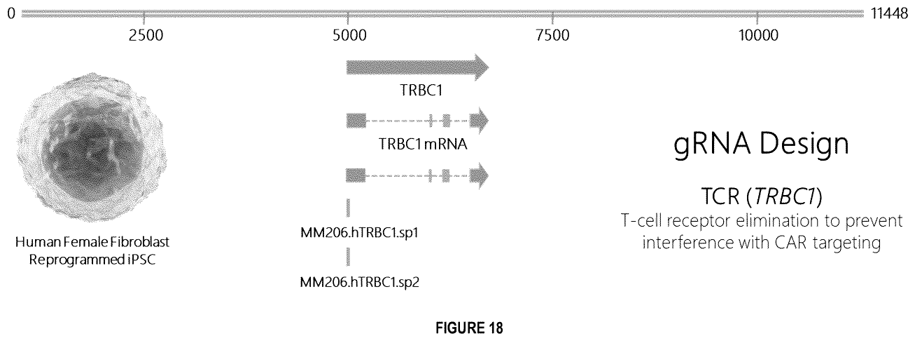

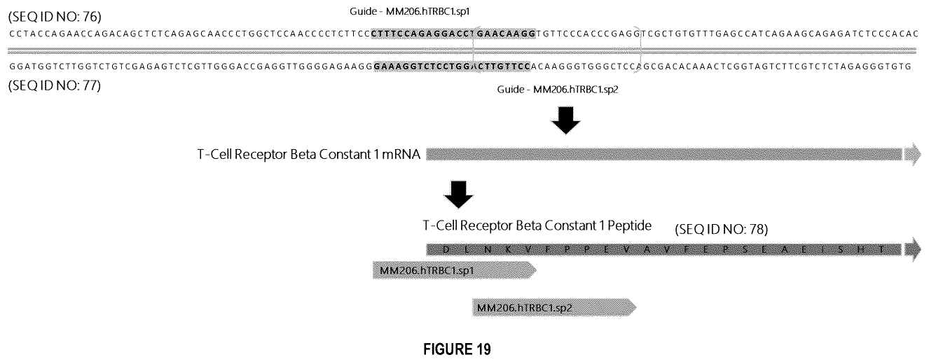

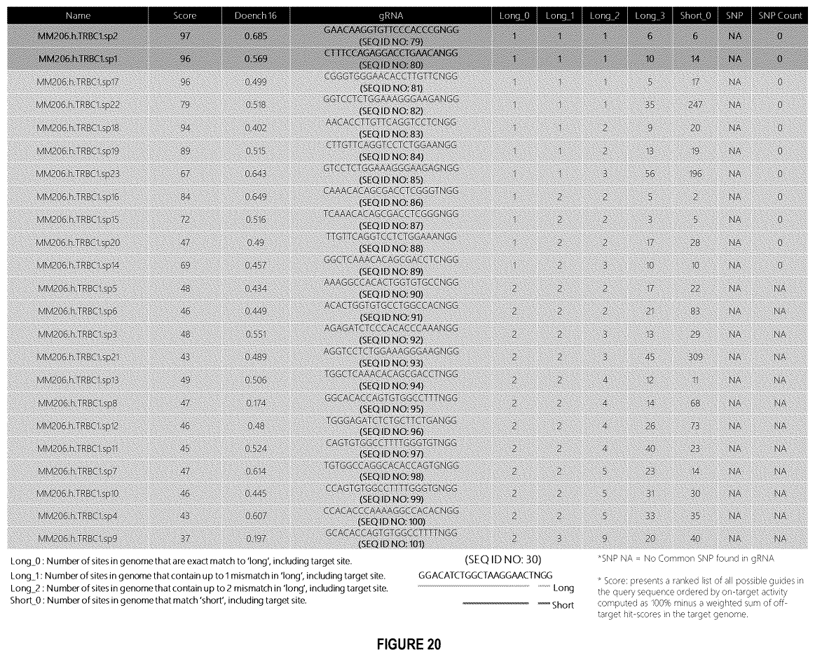

FIG. 18 depicts a graphical representation of a TRBC1 genetic knock-out strategy for hypoimmunogenic cell line development from human fibroblast reprogrammed iPSCs. In embodiments, the T cell receptor beta chain 1 (TRBC1) elimination is done to prevent interference with CAR targeting and off-target effects. FIG. 19 depicts a graphical representation of a TRAC gRNA design for hypoimmunogenic cell line development. The strategy, in embodiments, involves using CRISPR/Cas9 and a specifically designed gRNA to target relevant regions of the TRAC gene (e.g., SEQ ID NOs: 76-77), for example gRNAs of SEQ ID NOs: 79-101. FIG. 20 depicts a tabular representation of a TRAC gRNA off-target analysis for hypoimmunogenic cell line development.

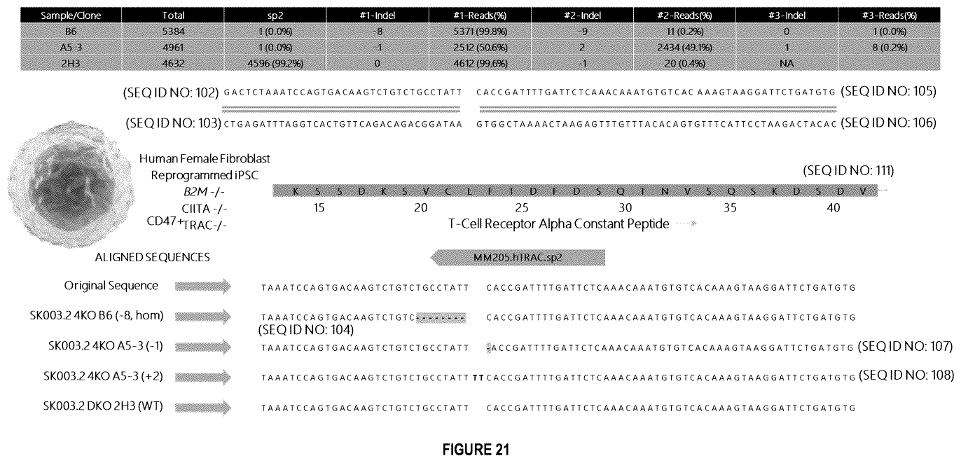

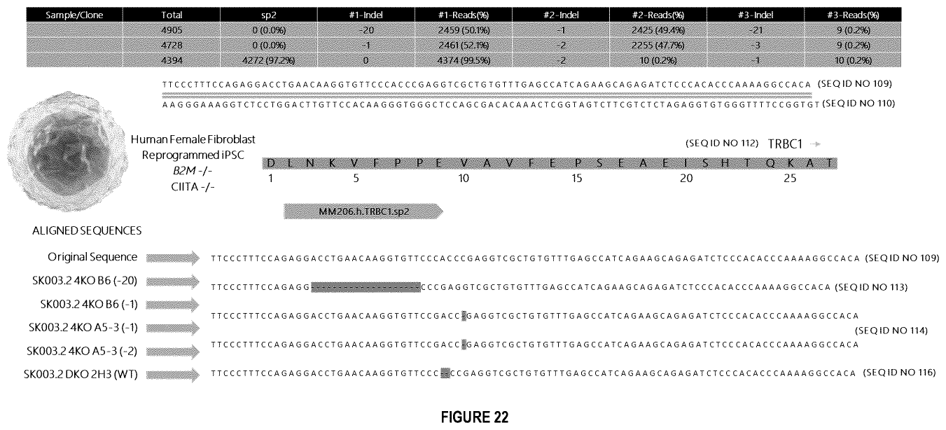

FIG. 21 depicts a graphical representation of a TRAC knock-out clonal sequence analysis for hypoimmunogenic cell line development for the B2M/CIITA double knock-out hCD47 knock-in human fibroblast reprogrammed iPSCs. The results for two selected clones are shown and the resultant sequences, e.g., SEQ ID NOs: 102-108. Sequence data illustrate successful TRAC knock-out in the double KO/CD47 KI clones. FIG. 22 depicts a graphical representation of a TRBC1 knock-out clonal sequence analysis for hypoimmunogenic cell line development for the B2M/CIITA double knock-out, hCD47 knock-in, and TRAC knock-out in human fibroblast reprogrammed iPSCs. The results for two selected clones are shown and the resultant sequences, e.g., SEQ ID NOs: 109-110 and 112-116.

FIG. 23 demonstrates successful biallelic disruption of B2M, CIITA, TRAC, and TRBC1 as evidenced by sequencing data. In addition, normal cellular morphology of the clones is maintained.

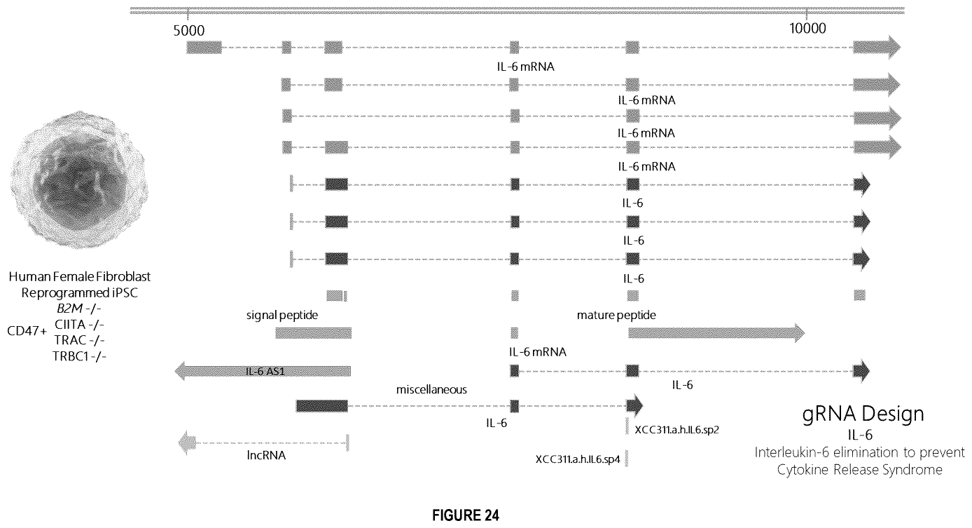

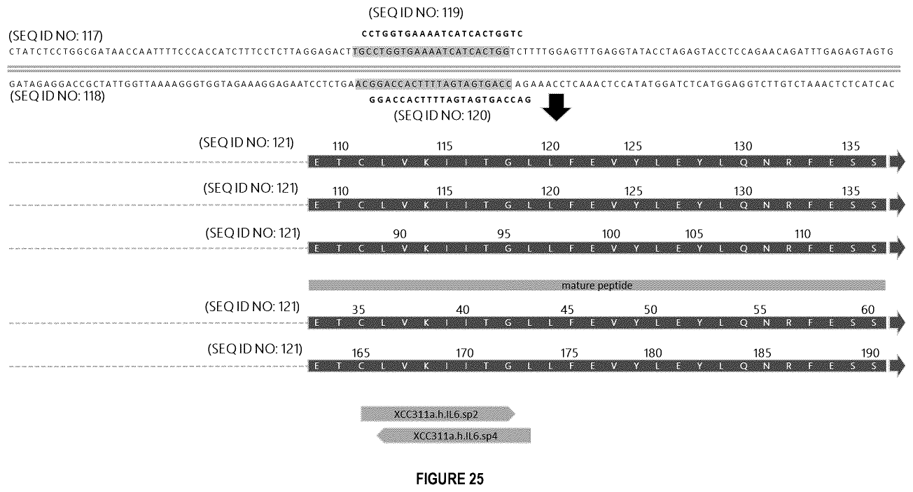

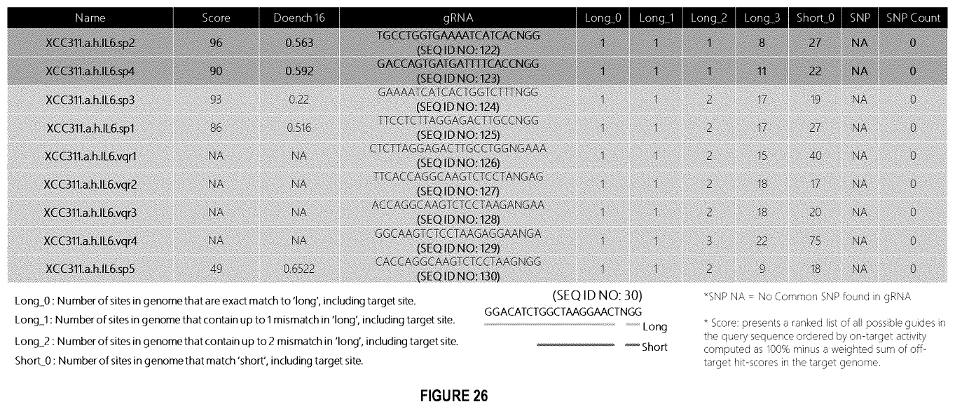

FIG. 24 depicts a graphical representation of an IL-6 genetic knock-out strategy for hypoimmunogenic cell line development from human fibroblast reprogrammed iPSCs. Interleukin-6 (IL-6) elimination is to precent Cytokine Release Syndrome (CRS). FIG. 25 depicts a graphical representation of an IL-6 gRNA design for hypoimmunogenic cell line development. The strategy, in embodiments, involves using CRISPR/Cas9 and a specifically designed gRNA to target relevant regions of the IL-6 gene (e.g., SEQ ID NOs: 117-118), for example gRNAs of SEQ ID NOs: 119-120 and 122-130. FIG. 26 depicts a tabular representation of an IL-6 gRNA off-target analysis for hypoimmunogenic cell line development.

TABLE 8
Sequences used for generating one or more of B2M KO, CIITA KO, TRAC KO, TRAC KO, TRBC1 KO,
IL-6 KO, and hCD47 isoform 2 KI to iPSCs.

| | |
|---|---|
| SEQ ID NO | Sequences |

B2M KO sequences

| | |
|---|---|
| SEQ ID NO: 21 | CTCGCGCTACTCTCTCTTTCTGGCCTGG |
| SEQ ID NO: 22 | GCCTTAGCTGTGCTCGCGCTACTCTCTCTTTCTGGCCTGGAGGCTATCCAGCGTGAGTCTC |
| | TCCTACCCTCCCGCTCTGGTCCTTCCTCTCCGCTCTGCACCCTCTGTGGCCCTCGCTGTGC |
| | TCTCTCG |
| SEQ ID NO: 23 | CGGAATCGACACGAGCGCGATGAGAGAGAAAGACCGGACCTCCCGATAGGTCGCACTCAG |
| | AGAGGATGGGAGGGCGAGACCAGGAAGGAGAGGCGAGACGTGGGAGACACCGGGAGCG |
| | ACACGAGAGAGC |
| SEQ ID NO: 131 | MSRSVALAVLALLSLSGLEAIQ |
| SEQ ID NO: 24 | GAGCGCGATGAGAGAGAAAGACCGGACC |
| SEQ ID NO: 25 | ALAVLALLSLSGLEAIQ |
| SEQ ID NO: 133 | GAGTAGCGCGAGCACAGCTANGG |
| SEQ ID NO: 134 | CTCGCGCTACTCTCTCTTTCNGG |
| SEQ ID NO: 26 | GCTACTCTCTCTTTCTGGCCNGG |
| SEQ ID NO: 27 | ACTCTCTCTTTCTGGCCTGGNGG |
| SEQ ID NO: 28 | CTACTCTCTCTTTCTGGCCTNGAG |
| SEQ ID NO: 29 | GCCAGAAAGAGAGAGTAGCGNGAG |
| SEQ ID NO: 30 | GGACATCTGGCTAAGGAACTNGG |

CIITA KO Sequences

| | |
|---|---|
| SEQ ID NO: 31 | CCAGAGGAGCTTCCGGCAGACCT |
| SEQ ID NO: 32 | CTGCAGCCTTCCCAGAGGAGCTTCCGGCAGACCTGAAGCACTGGAAGCCAGGTGTGCAGG |
| | GCAGGTGGGCTGGGGTTGGGAAGGGTGGATGCCTTGGGGAGGGGATGGAAGAGATTTGA |
| | ACTCCTGGCCCA |
| SEQ ID NO: 33 | GACGTCGGAAGGGTCTCCTCGAAGGCCGTCTGGACTTCGTGACCTTCGGTCCACACGTCC |
| | CGTCCACCCGACCCCAACCCTTCCCACCTACGGAACCCTCCCCTACCTTCTCTAAACTTGA |
| | GGACCGGGT |
| SEQ ID NO: 34 | GGTCTCCTCGAAGGCCGTCTGGA |
| SEQ ID NO: 35 | PFPEELPADLKHYKP |
| SEQ ID NO: 36 | CCAGAGGAGCTTCCGGCAGACCT |
| SEQ ID NO: 37 | GGTCTGCCGGAAGCTCCTCTNGG |
| SEQ ID NO: 38 | GACCTGAAGCACTGGAAGCCNGG |
| SEQ ID NO: 39 | TTCCCAGAGGAGCTTCCGGCNGAC |
| SEQ ID NO: 40 | AGAGGAGCTTCCGGCAGACCNGAA |
| SEQ ID NO: 41 | TCCGGCAGACCTGAAGCACTNGAA |
| SEQ ID NO: 42 | TGCCGGAAGCTCCTCTGGGANGG |

B2M Clonal Sequences

| | |
|---|---|
| SEQ ID NO: 43 | CGAGATGTCTCGCTCCGTGGCCTTAGCT |
| SEQ ID NO: 44 | GCTCTACAGAGCGAGGCACCGGAATCGA |
| SEQ ID NO: 45 | CGAGATGTCTCGCTCCGTGGCCTTAG |
| SEQ ID NO: 48 | GTGCTCGCGCTACTCTCTCTTTCTGGCCTGGAGGCTATCCAGCGTGAG |
| SEQ ID NO: 49 | CACGAGCGCGATGAGAGAGAAAGACCGGACCTCCGATAGGTCGCACTC |

CIITA Clonal Sequences

| | |
|---|---|
| SEQ ID NO: 50 | CACATGTTTTCTCTGCAGCCTTCCCAGAGGAGCTTCCGGCAGACCTGAAGCACTGGAAGCC |
| | AGGTGTGCAGGGCAGGTGGGCTGGGGTTGGG |
| SEQ ID NO: 51 | GTGTACAAAAGAGACGTCGGAAGGGTCTCCTCGAAGGCCGTCTGGACTTCGTGACCTTCG |
| | GTCCACACGTCCCGTCCCACCCGACCCCAACCC |
| SEQ ID NO: 52 | CACATGTTTTCTCTGCAGCCTTCCCAGAGCAGACCTGAAGCACTGGAAGCCAGGTGTGCAG |
| | GGCAGGTGGGCTGGGGTTGGG |
| SEQ ID NO: 53 | CACATGTTTTCTCTGCAGCAGCTTCCGGCAGACCTGAAGCACTGGAAGCCAGGTGTGCAG |
| | GGCAGGTGGGCTGGGGTTGGG |
| SEQ ID NO: 54 | CACATGTTTTCTCTGCAGCCTTCCCAGAGAGCTTCCGGCAGACCTGAAGCACTGGAAGCCA |
| | GGTGTGCAGGGCAGGTGGGCTGGGGTTGGG |
| SEQ ID NO: 55 | CACATGTTTTCTCTGCAGCCTTCCGGCAGACCTGAAGCACTGGAAGCCAGGTGTGCAGGG |
| | CAGGTGGGCTGGGGTTGGG |

TRAC KO Sequences

| | |
|---|---|
| SEQ ID NO: 56 | TGGGTTGGGGCAAAGAGGGAAATGAGATCATGTCCAACCCTGATCCTCTTGTCCCACAGAT |
| | ATCCAGAACCCTGACCCTGCCGTGTACCAGCTGAGAGACTCTAAATCCAGTGACAAGTCTG |
| | TCTGCCTATTCACCGATTTTGATTCTCAAACAAATGTGTCACAAAGTAAGGATTCTGATGTGT |
| | ATATCACAGACAAAACTGTGCTAGACATGAGGTCTATGGACTTCAAGAGCAACAGTGCTGT |
| | GGCCTGGAGCAAC |
| SEQ ID NO: 57 | ACCCAACCCCGTTTCTCCCTTTACTCTAGTACAGGATTGGGACTAGGAGAACAGGGTGTCT |
| | ATAGGTCTTGGGACTGGGACGGCACATGGTCGACTCTCTGAGATTTAGGTCACTGTTCAGA |
| | CAGACGGATAAGTGGCTAAAACTAAGAGTTTGTTTACACAGTGTTTCATTCCTAAGACTACA |
| | CATATAGTGTCTGTTTTGACACGATCTGTACTCCAGATACCTGAAGTTCTCGTTGTCACGAC |
| | ACCGGACCTCGTTG |
| SEQ ID NO: 132 | IQNPDPAVYQLRDSKSSDKSVCLFTDFDSQTNVSQSKDSDVYITDKTVLDMRSMDFKSNSAVA |
| | WSN |
| SEQ ID NO: 60 | TCAGGGTTCTGGATATCTGTNGG |
| SEQ ID NO: 61 | GAGAATCAAAATCGGTGAATNGG |
| SEQ ID NO: 62 | TGGATTTAGAGTCTCTCAGCNGG |
| SEQ ID NO: 63 | TCTCTCAGCTGGTACACGGCNGG |
| SEQ ID NO: 64 | ACACGGCAGGGTCAGGGTTCNGG |
| SEQ ID NO: 65 | GCTGGTACACGGCAGGGTCANGG |
| SEQ ID NO: 66 | ATCTGTGGGACAAGAGGATCNGG |
| SEQ ID NO: 67 | GTCAGGGTTCTGGATATCTGNGG |
| SEQ ID NO: 68 | CTCTCAGCTGGTACACGGCANGG |
| SEQ ID NO: 69 | CTGGATATCTGTGGGACAAGNGG |
| SEQ ID NO: 70 | AGCTGGTACACGGCAGGGTCNGG |
| SEQ ID NO: 71 | TCTGTGGGACAAGAGGATCANGG |
| SEQ ID NO: 72 | AGAGTCTCTCAGCTGGTACANGG |
| SEQ ID NO: 73 | ATTTGTTTGAGAATCAAAATNGG |
| SEQ ID NO: 74 | TAGGCAGACAGACTTGTCACNGG |
| SEQ ID NO: 75 | GGGACAAGAGGATCAGGGTTNGG |

TRBC1 KO Sequences

| | |
|---|---|
| SEQ ID NO: 46 | CGAGATGTCTCGCTCCGTGGCCTTAGCTCT |
| SEQ ID NO: 47 | CGAGATGTCTCGCTCCGTGGCCT |
| SEQ ID NO: 76 | CCTACCAGAACCAGACAGCTCTCAGAGCAACCCTGGCTCCAACCCCTCTTCCCTTTCCAGA |
| | GGACCTGAACAAGGTGTTCCCACCCGAGGTCGCTGTGTTTGAGCCATCAGAAGCAGAGAT |
| | CTCCCACAC |
| SEQ ID NO: 77 | GGATGGTCTTGGTCTGTCGAGAGTCTCGTTGGGACCGAGGTTGGGGAGAAGGGAAAGGTC |
| | TCCTGGACTTGTTCCACAAGGGTGGGCTCCAGCGACACAAACTCGGTAGTCTTCGTCTCTA |
| | GAGGGTGTG |
| SEQ ID NO: 78 | DLNKVFPPEVAVFEPSEAEISHT |
| SEQ ID NO: 79 | GAACAAGGTGTTCCCACCCGNGG |
| SEQ ID NO: 80 | CTTTCCAGAGGACCTGAACANGG |
| SEQ ID NO: 81 | CGGGTGGGAACACCTTGTTCNGG |
| SEQ ID NO: 82 | GGTCCTCTGGAAAGGGAAGANGG |
| SEQ ID NO: 83 | AACACCTTGTTCAGGTCCTCNGG |
| SEQ ID NO: 84 | CTTGTTCAGGTCCTCTGGAANGG |
| SEQ ID NO: 85 | GTCCTCTGGAAAGGGAAGAGNGG |
| SEQ ID NO: 86 | CAAACACAGCGACCTCGGGTNGG |
| SEQ ID NO: 87 | TCAAACACAGCGACCTCGGGNGG |
| SEQ ID NO: 88 | TTGTTCAGGTCCTCTGGAAANGG |
| SEQ ID NO: 89 | GGCTCAAACACAGCGACCTCNGG |
| SEQ ID NO: 90 | AAAGGCCACACTGGTGTGCCNGG |
| SEQ ID NO: 91 | ACACTGGTGTGCCTGGCCACNGG |
| SEQ ID NO: 92 | AGAGATCTCCCACACCCAAANGG |
| SEQ ID NO: 93 | AGGTCCTCTGGAAAGGGAAGNGG |
| SEQ ID NO: 94 | TGGCTCAAACACAGCGACCTNGG |
| SEQ ID NO: 95 | GGCACACCAGTGTGGCCTTTNGG |
| SEQ ID NO: 96 | TGGGAGATCTCTGCTTCTGANGG |
| SEQ ID NO: 97 | CAGTGTGGCCTTTTGGGTGTNGG |
| SEQ ID NO: 98 | TGTGGCCAGGCACACCAGTGNGG |
| SEQ ID NO: 99 | CCAGTGTGGCCTTTTGGGTGNGG |
| SEQ ID NO: 100 | CCACACCCAAAAGGCCACACNGG |
| SEQ ID NO: 101 | GCACACCAGTGTGGCCTTTTNGG |

TRAC Clonal Sequences

| | |
|---|---|
| SEQ ID NO: 102 | GACTCTAAATCCAGTGACAAGTCTGTCTGCCTATT 102 |
| SEQ ID NO: 103 | CTGAGATTTAGGTCACTGTTCAGACAGACGGATAA 103 |
| SEQ ID NO: 104 | TAAATCCAGTGACAAGTCTGTC 104 |
| SEQ ID NO: 105 | CACCGATTTTGATTCTCAAACAAATGTGTCACAAAGTAAGGATTCTGATGTG 105 |
| SEQ ID NO: 106 | GTGGCTAAAACTAAGAGTTTGTTTACACAGTGTTTCATTCCTAAGACTACAC 106 |
| SEQ ID NO: 107 | ACCGATTTTGATTCTCAAACAAATGTGTCACAAAGTAAGGATTCTGATGTG 107 |
| SEQ ID NO: 108 | TTCACCGATTTTGATTCTCAAACAAATGTGTCACAAAGTAAGGATTCTGATGTG 108 |
| SEQ ID NO: 111 | KSSDKSVCLFTDFDSQTNVSQSKDSDV |

TRBC1 Clonal Sequences

| | |
|---|---|
| SEQ ID NO: 109 | TTCCCTTTCCAGAGGACCTGAACAAGGTGTTCCCACCCGAGGTCGCTGTGTTTGAGCCATC |
| | AGAAGCAGAGATCTCCCACACCCAAAAGGCCACA |
| SEQ ID NO: 110 | AAGGGAAAGGTCTCCTGGACTTGTTCCACAAGGGTGGGCTCCAGCGACACAAACTCGGTA |
| | GTCTTCGTCTCTAGAGGTGTGGGTTTTCCGGTGT |
| SEQ ID NO: 112 | DLNKVFPPEVAVFEPSEAEISHTQKAT |
| SEQ ID NO: 113 | TTCCCTTTCCAGAGGCCCGAGGTCGCTGTGTTTGAGCCATCAGAAGCAGAGATCTCCCACA |
| | CCCAAAAGGCCACA |
| SEQ ID NO: 114 | TTCCCTTTCCAGAGGACCTGAACAAGGTGTTCCGACCGAGGTCGCTGTGTTTGAGCCATCA |
| | GAAGCAGAGATCTCCCACACCCAAAAGGCCACA |
| SEQ ID NO: 116 | TTCCCTTTCCAGAGGACCTGAACAAGGTGTTCCCCCGAGGTCGCTGTGTTTGAGCCATCAG |
| | AAGCAGAGATCTCCCACACCCAAAAGGCCACA |

IL-6 KO Sequences

| | |
|---|---|
| SEQ ID NO: 117 | CTATCTCCTGGCGATAACCAATTTTCCCACCATCTTTCCTCTTAGGAGACTTGCCTGGTGAA |
| | AATCATCACTGGTCTTTTGGAGTTTGAGGTATACCTAGAGTACCTCCAGAACAGATTTGAGA |
| | GTAGTG |
| SEQ ID NO: 118 | GATAGAGGACCGCTATTGGTTAAAAGGGTGGTAGAAAGGAGAATCCTCTGAACGGACCACT |
| | TTTAGTAGTGACC |
| SEQ ID NO: 119 | CCTGGTGAAAATCATCACTGGTC |
| SEQ ID NO: 120 | GGACCACTTTTAGTAGTGACCAG |
| SEQ ID NO: 121 | ETCLVKIITGLLFEVYLEYLQNRFESS |
| SEQ ID NO: 122 | TGCCTGGTGAAAATCATCACNGG |
| SEQ ID NO: 123 | GACCAGTGATGATTTTCACCNGG |
| SEQ ID NO: 124 | GAAAATCATCACTGGTCTTTNGG |
| SEQ ID NO: 125 | TTCCTCTTAGGAGACTTGCCNGG |
| SEQ ID NO: 126 | CTCTTAGGAGACTTGCCTGGNGAAA |
| SEQ ID NO: 127 | TTCACCAGGCAAGTCTCCTANGAG |
| SEQ ID NO: 128 | ACCAGGCAAGTCTCCTAAGANGAA |
| SEQ ID NO: 129 | GGCAAGTCTCCTAAGAGGAANGA |
| SEQ ID NO: 130 | CACCAGGCAAGTCTCCTAAGNGG |

### Example 2: Generating BioNVs Via Serial Extrusion

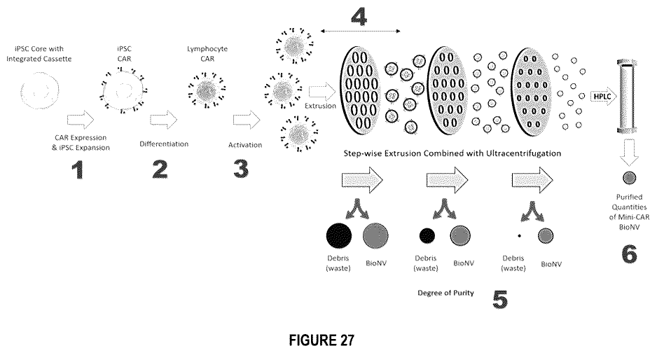

Biomimetic Nanovesicles (BioNVs) can be produced from the above hypoimmunogenic cell lines as illustrated in the scheme depicted in FIG. 27.

The level of CAR expression can be measured in the hypoimmunogenic cell line using a combination of flow cytometry and iodixanol density gradient (e.g., STEP 1 of FIG. 27).

The differentiation of the iPSC-expressing surface CAR into CAR-lymphocytes can be analyzed by lymphocyte marker identification including, for example CD4/CD8 (T-cells) or CD56/CD16 (Natural Killers cells), among other cell surface markers (e.g., STEP 2 of FIG. 27). The expression profile can be determined via flow cytometry, RT-PCR, and/or CRISPR-based analytics.

Next, the activation of the CAR lymphocytes can be achieved using biomarker antigen-coated beads in low, pre-determined concentrations over the course of two weeks in two stages (e.g., STEP 3 of FIG. 27). This process can also analyze the quality of the Immunological Synapse (IS) between the CAR and the antigen-coated beads, using well-established protocols to measure i) the quantification of F-Actin accumulation at the site of synapse formation, ii) the distribution of pZeta at synapse, iii) the clustering of an antigen through the IS location, and/or iv) the polarization of lytic granules (LGs) that contain perforin and granzymes.

After the activation of the lymphocytes, the cells are expanded using established protocols (e.g., STEP 4 of FIG. 27). After expansion, the levels of perforin and granzyme (or other lumen payloads if applicable) are analyzed per cell population to ensure consistent concentration levels on a per-batch basis. This is accomplished using a series of qPCR, immunoblotting, flow cytometry, and/or mass spectrometry. The expansion step may not be necessary if a large enough cell population from Step 3 can be achieved.

Once the cells are activated to produce the desired therapeutic protein(s), they are expanded, harvested, washed several times, and then placed into a buffered extrusion medium. The cells are then wholly processed via serial extrusion through each step of the polycarbonate filter system that consists of diminishing pore size (e.g., STEP 5 of FIG. 27). In the initial extrusion step of the serial extrusion process, the nucleus (along with nuclear components including nuclear pores, genomic material, and transcription factors) and mitochondria are eliminated. The sample is then treated with endonuclease, e.g., BENZONASE. BENZONASE is a non-specific, recombinant endonuclease that cleaves all types of DNA and RNA variants into non-functional fragments <8 soluble base pairs. This leads to the highest reduction of nucleic acid load on a per sample and scalable basis and does not interfere with BioNV membrane chemistry. The cleavage process also eliminates nucleic viscosity, allowing for subsequent loading and passage of materials through the next set of extrusion filters. There is an FDA regulatory guideline in place for the use of BENZONASE in the manufacturing of vaccines that is applicable to the extrusion process of BioNVs.

The serial extrusion process will avoid the elimination of other organelles such as the Golgi Apparatus or the ER. The membrane system of these organelles is highly evolved to traffic vesicles (release and uptake) between folded membranes. For example, the cis and trans face of the Golgi Apparatus contain unique lipid compositions that facilitate low energy barrier absorption and release in the trafficking of vesicles. These components are relatively low in the cytoplasmic membrane. Therefore, isolating the cytoplasmic membrane for BioNV derivation is not as favorable. As the BioNVs are passed through the polycarbonate filters in the serial extrusion process, they undergo destruction and spontaneous formation based on the pore size. This process results in BioNVs containing membranes with a homogenous mixture of cytoplasm, Golgi, and ER lipid content and protein components that can considerably increase their affinity for cellular and tissue delivery uptake in comparison to BioNVs processed to eliminate these organelles. These features could translate to better and more consistent uptake of BioNVs into targeted cells at much lower doses than systems that do not incorporate these properties.

After the extrusion step, the BioNVs are passed through an α-CD3 HPLC (FPLC in scale-up) column to remove the low percent (approximately 0.05%) of inverted BioNVs that spontaneously form during the serial extrusion process (e.g., STEP 6 of FIG. 27). This is done to ensure the resultant BioNVs have homogenous directionality with respect to the membranes. Low loss of yield occurs during this step, as it is a flow-through process to capture impurities. Once the BioNVs have been collected after the HPLC/FPLC step, they are tested through a standardization process.

The standardization process includes one or more the following assays:
BioNV homogeneity: the use of Nanoparticle Flow Cytometry (NanoFCM) can confirm BioNV concentration, homogeneity of size, the density of the BioNVs, and/or the homogeneity of the BioNV lumen constituents.
Concentration of lumen payload: NanoFM technology can be used to determine the type and concentration of the nucleic acids/proteins that are packaged into the lumen of the BioNVs. These data can be confirmed in parallel with one or more methods including immunoblot, mass spectrometry, and BCA analyses to determine the nucleic acid and protein content of BioNVs.
BioNV Stability: a combination of nanoparticle tracking analysis (NTA), dynamic light scattering (DLS), and electron microscopy (EM) can be used in combination with immunoblot and mass spectrometry analyses to determine the physical and biochemical features of the BioNVs over 8 to 10 months. Data from these assays can include the protein expression profile, the degree of intact BioNV membranes/packaging, and/or the degree of aggregation.
Membrane Integrity: the integrity of the BioNV membranes is evaluated using calcein release assays combined with NanoFCM to assess membrane permeability. The results can provide insight into the leakage properties of the BioNVs against standardized BioNV panels.
Quality of lumen payload: the quality of the lumen-packaged payloads can be determined using multiple analytic assays, depending on the nature of the payload. In instances where the deliverable is a nucleic acid, qPCR and/or sequencing over 8 to 10 months can be used to check the integrity and quantity of the nucleic acid payloads. For proteins, an analysis of the BioNV constituents using one or more of NanoFCM, mass spectrometry, and immunoblot analyses can be used to analyze the protein payload.
CAR Quality and Surface Density: CAR surface density can be determined using NanoFCM, mass spectrometry, and/or immunoblot analyses. CAR surface density is expected to be at least about 5-fold to at least about 10-fold higher in BioNVs compared to whole cell surface densities. This could considerably enhance targeting to the antigen in comparison to a whole cell. CAR quality can be determined at the cellular stage, as described above (e.g., as in STEP 3). A mathematical model can be used to extrapolate cellular quality data and apply it to the BioNVs in relation to efficacy study data outcomes.
BioNV Functionality: BioNVs can be tested for basic functionality, including multiple and defined standardization assays, such as in vitro cellular uptake into targeted cells with and without expressed antigen, as well their ability to cross dense tissues such as those in human retinal models. Following these basic functionality assays, which can be performed immediately after the serial extrusion process, pre-clinical studies will address the remainder of the quality and functionality properties of the BioNVs.

### DEFINITIONS

The following definitions are used in connection with the disclosure herein. Unless defined otherwise, all technical and scientific terms used herein have the same meaning as commonly understood to one of skill in the art to which this disclosure belongs.

An “effective amount,” or “therapeutically effective amount,” is an amount that is effective for treating, preventing, or ameliorating a mammalian disease.

As used herein, “a,” “an,” or “the” can mean one or more than one.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosed methods and compositions, the present disclosure, or embodiments thereof, and may alternatively be described using alternative terms such as “consisting of” or “consisting essentially of.”

In embodiments, a “BioNV” refers to biomimetic nanovesicles (NVs) which encapsulate an aqueous compartment. In embodiments, BioNVs are allogeneic and/or hypoimmune. In embodiments, BioNV can refer to NVs made by rupturing/disrupting the cell (e.g., cell-derived vesicles), or nanovesicles which are naturally shed from the cell (e.g., exosomes). In embodiments, BioNVs comprise at least one surface-oriented, membrane-embedded CARs. In embodiments, “nanovesicles (NVs),” as referred to herein, are lipid-bound vesicles on the order of about 10 nm to about 1200 nm in size which encapsulate an aqueous core. In embodiments, lipid bound NVs can form using lipid monolayers, lipid bilayers, or maintain multilamellar forms. In embodiments, BioNV refers to biologically derived nano-sized vesicles that can have designed biological functionalization. In embodiments, BioNVs are “biomimetic” in that they are derived from endogenous cellular material, more specifically, they substantially recapitulate plasma membrane material found in cells. In embodiments, the cells from which BioNVs originate can include stem cells of any kind, including cell types differentiated from said stem cells. In embodiments, BioNVs are substantially free of encapsulated cellular debris including nucleic acid, organelles, or organelle parts. In embodiments, BioNVs are characterized as having one or more, 2 or more, 3 or more, 4 or more, 5 or more, or 6 or more of the following:
a. being about 10 nm to about 1200 nm in size;
b. having a total volume of about 500 nm³ to about 5 μm³ (assuming spherical shape);
c. having a content of at least one phospholipid and cholesterol;
d. having a surface membrane having one or more of CD34, CCL21, PD-L1 (in BioNVs derived from non-activated cell sources), FasL, SerpinB9, H2-M3, CD47, CTLA-4, CD24, CD200, MFG-E8, NCAM, α-phagocytic integrin, and/or anti-6R antibody or antibody format, or a chimera of any one or more thereof; having a surface membrane substantially lacking T cell receptor components (TRAC and/or TRBC), MHC class I components, and/or MHC class II components, lacking proteins of one or more of HLA-A, HLA-B, HLA-C, HLA-E or HLA-G (but not both of HLA-E and HLA-G), HLA-F, and/or CIITA, SerpinB9, substantially lacking proteins inside the vesicle of one or more of IL-4, IL-6, IL-10, and/or IL-16;
e. encapsulating one or more therapeutically relevant biomolecules, including for example, a cytokine including chemokines, interferons (IFNα/β/γ), interleukins, alarmins, lymphokines, tumor necrosis factors (TNFs), colony-stimulating factors, bone morphogenic protein (BMP), erythropoietin (EPO), granulocyte-stimulating factor (G-CSF), granulocyte macrophage colony-stimulating factor (GM-CSF), pro-inflammatory cytokines, anti-antigen cytokines, perforin granzyme (e.g., granzyme A, B, H, K, and M), gene editing payloads, fusion proteins, antibodies or antibody format constructs, or a combination thereof.
f. having a membrane-embedded targeting agent comprising a target-binding moiety which can include an antibody or antibody format selected from one or more of a CAR, monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, fusion protein comprising the antigen-binding portion of an antibody, Bispecific T cell Engagers (BiTE), viral epitope recognition receptor (VERR) or viral ligand, a variable heavy chain IgG fragment V_{H}H or V_{NAR} or through a T-Cell Receptor (TCR), wherein the CAR can target a single biomarker or multiple biomarkers, or multiple parts of a single biomarker, the one or more targeting agents can include a ligand for a receptor or a receptor for a ligand;
   g. being capable of adsorbing and/or encapsulating a payload of one or more perforins, granzymes, cytokines, cytotoxic proteins, non-naturally occurring cellular agents, checkpoint inhibiting agents, recombinant gene editing payloads, antibodies or antibody fragments, small molecule inhibitors, biologics, radionuclides, tracing agents, dyes, fluorescent proteins, among other therapeutic payloads, and/or any combination thereof; and
   h. being capable of not causing a deleterious immune reaction in subjects.

In embodiments, “induced pluripotent stem cells,” or “iPSCs” refers to stem cells that can be generated directly from adult cells. iPSCs can originate from differentiated cells that are reprogrammed back into an embryonic-like pluripotent state. iPSCs can generally propagate indefinitely and become any cell type of the organism they originate.

In embodiments, “allogeneic,” as used herein, refers to biological material, tissues, or cells, which are genetically dissimilar and originally immunological incompatible, despite originating from the same species. Allogeneic CDV/exosomes, for example, are material that originates from a first subject (iPSC donor) and can be provided to any number of distinct subjects who are not genetically identical.

In embodiments, “hypoimmunogenic” or “hypoimmune,” as used herein in reference to a cell and/or BioNV, refers to a reduced capacity to generate an immunological response. In embodiments, cells and BioNVs can be hypoimmunogenic due to reduced or ablated expression of one or more specific cell surface proteins and/or secreted proteins, such as T cell receptor (TCR) proteins, cytokine response syndrome proteins, MHC class I or II proteins, etc. In embodiments, cells and BioNVs can be hypoimmunogenic due to increased immunoprotective cell surface proteins, such as CD47, CD34, CD24, CD200, α-phagocytic integrins, etc. In embodiments, BioNVs and/or cells can be hypoimmunogenic due to not triggering CRS in a subject and/or not inducing HLA incompatibility.

In embodiments, “knocking-out,” “silencing,” “inactivating,” “disrupting,” or “blocking,” and their equivalencies, with respect to transcription, gene expression, or protein expression, refers to an amount of transcription, gene or protein expression which is reduced from a normal state or less than the wild-type state in a particular cell subset. The reduction can be significant so that no gene expression occurs, or a negligible amount of expression occurs.

In embodiments, “overexpression,” as used herein, refers to an amount of transcription, gene or protein expression which is increased from a normal state or more than the wild-type state in a particular cell subset.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

### INCORPORATION BY REFERENCE

All patents and publications referenced herein are hereby incorporated by reference in their entireties, including published PCT application, WO 2020/227369, filed May 6, 2020, titled “Tailored Hypoimmune Nanovesicle Delivery Systems for Cancer Tumors,” and published U.S. non-provisional application, US20220040106 A1, filed Aug. 3, 2021, titled “Tailored Hypoimmune Nanovesicular Delivery Systems for Cancer Tumors, Hereditary and Infectious Diseases.”

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this disclosure pertains.

The disclosure has been described in an illustrative manner, and it is to be understood that the terminology which has been used is intended to be in the nature of words of description rather than of limitation.

Obviously, many modifications and variations of the present disclosure are possible in light of the above teachings. It is, therefore, to be understood that within the scope of the appended claims, the disclosure can be practiced otherwise than as specifically described.

What is claimed is:

1. A method of generating a hypoimmunogenic cell comprising:
(a) reducing or ablating the expression and/or activity of one or more immunogenic proteins in a cell; and
(b) expressing or increasing expression and/or activity of one or more immunoprotective proteins in the cell, thereby generating the hypoimmunogenic cell.

2. The method of claim 1, wherein the cell is a stem cell, an induced pluripotent stem cell (iPSC), a reprogrammed pluripotent or multipotent cell, an embryonic stem cell, a mesenchymal stem cell, or a differentiated cell which originates from any stem cell thereof.

3. The method of claim 2, wherein the differentiated cell is a T cell, helper T cell, T-memory cell, or NK cell.

4. The method of claim 2, wherein the differentiated cell is a macrophage.

5. The method of claim 2, wherein the differentiated cell is a monocyte.

6. The method of claim 2, wherein the differentiated cell is a hepatocyte, cardiomyocyte, neuron, endothelial cell, pancreatic cell, or retinal pigmented epithelium (RPE) cell.

7. The method of any one of the preceding claims, wherein the hypoimmunogenic cell substantially lacks one or more MHC class I proteins, MHC class II proteins, T cell receptor (TCR) proteins, and/or cytokine release syndrome (CRS) proteins.

8. The method of any one of the preceding claims, wherein reducing or ablating the expression and/or activity of one or more immunogenic proteins comprises a β2-macroglobulin (B2M) gene disruption and/or a disruption that reduces or ablates MHC class I protein expression and/or activity.

9. The method of any one of the preceding claims, wherein reducing or ablating the expression and/or activity of one or more immunogenic proteins comprises a CIITA gene disruption and/or a disruption that reduces or ablates MHC class II protein expression and/or activity.

10. The method of any one of the preceding claims, wherein reducing or ablating the expression and/or activity of one or more immunogenic proteins comprises an HLA-A gene disruption and/or a disruption that reduces or ablates HLA-A protein expression and/or activity.

11. The method of any one of the preceding claims, wherein reducing or ablating the expression and/or activity of one or more immunogenic proteins comprises an HLA-B gene disruption and/or a disruption that reduces or ablates HLA-B protein expression and/or activity.

12. The method of any one of the preceding claims, wherein reducing or ablating the expression and/or activity of one or more immunogenic proteins comprises an HLA-C gene disruption and/or a disruption that reduces or ablates HLA-C protein expression and/or activity.

13. The method of any one of the preceding claims, wherein reducing or ablating the expression and/or activity of one or more immunogenic proteins comprises an HLA-E gene disruption or an HLA-G gene disruption and/or a disruption that reduces or ablates HLA-E or HLA-G protein expression and/or activity.

14. The method of any one of the preceding claims, wherein reducing or ablating the expression and/or activity of one or more immunogenic proteins comprises an HLA-F gene disruption and/or a disruption that reduces or ablates HLA-F protein expression and/or activity.

15. The method of any one of the preceding claims, wherein reducing or ablating the expression and/or activity of one or more immunogenic proteins comprises a T cell alpha constant (TRAC) gene disruption and/or a disruption that reduces or ablates TRAC protein expression and/or activity.

16. The method of any one of the preceding claims, wherein reducing or ablating the expression and/or activity of one or more immunogenic proteins comprises a T cell beta constant (TRBC) gene disruption and/or a disruption that reduces or ablates TRBC protein expression and/or activity.

17. The method of any one of the preceding claims, wherein reducing or ablating the expression and/or activity of one or more immunogenic proteins comprises a PD-1 gene disruption and/or a disruption that reduces or ablates PD-1 protein expression and/or activity.

18. The method of any one of the preceding claims, wherein reducing or ablating the expression and/or activity of one or more immunogenic proteins comprises an IL-4 gene disruption and/or a disruption that reduces or ablates IL-4 protein expression and/or activity.

19. The method of any one of the preceding claims, wherein reducing or ablating the expression and/or activity of one or more immunogenic proteins comprises an IL-6 gene disruption and/or a disruption that reduces or ablates IL-6 protein expression and/or activity.

20. The method of any one of the preceding claims, wherein reducing or ablating the expression and/or activity of one or more immunogenic proteins comprises an IL-10 gene disruption and/or a disruption that reduces or ablates IL-10 protein expression and/or activity.

21. The method of any one of the preceding claims, wherein reducing or ablating the expression and/or activity of one or more immunogenic proteins comprises an IL-16 gene disruption and/or a disruption that reduces or ablates IL-16 protein expression and/or activity.

22. The method of any one of the preceding claims, wherein reducing or ablating the expression and/or activity of one or more immunogenic proteins comprises a SerpinB9 gene disruption and/or a disruption that reduces or ablates SerpinB9 protein expression activity.

23. The method of any one of the preceding claims, wherein reducing or ablating the expression and/or activity of one or more immunogenic proteins is at the DNA level by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, small RNA, or a combination thereof.

24. The method of claim 23, wherein the small RNA is or comprises one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

25. The method of any one of the preceding claims, wherein reducing or ablating the expression and/or activity of one or more immunogenic proteins is at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

26. The method of any one of the preceding claims, wherein expressing or increasing expression and/or activity of one or more immunoprotective proteins comprises expressing or increasing expression of a CD34 gene and/or gene product.

27. The method of any one of the preceding claims, wherein expressing or increasing expression and/or activity of one or more immunoprotective proteins comprises expressing or increasing expression of a CCL2 gene and/or gene product.

28. The method of any one of the preceding claims, wherein expressing or increasing expression and/or activity of one or more immunoprotective proteins comprises expressing or increasing expression of a PD-L1 gene and/or gene product, and wherein the cell is not activated.

29. The method of any one of the preceding claims, wherein expressing or increasing expression and/or activity of one or more immunoprotective proteins comprises expressing or increasing expression of a H2-M3 gene and/or gene product.

30. The method of any one of the preceding claims, wherein expressing or increasing expression and/or activity of one or more immunoprotective proteins comprises expressing or increasing expression of a CD47 gene and/or gene product.

31. The method of any one of the preceding claims, wherein expressing or increasing expression and/or activity of one or more immunoprotective proteins comprises expressing or increasing expression of a CD24 gene and/or gene product.

32. The method of any one of the preceding claims, wherein expressing or increasing expression and/or activity of one or more immunoprotective proteins comprises expressing or increasing expression of a chimeric CD24/CD47 gene and/or gene product.

33. The method of any one of the preceding claims, wherein expressing or increasing expression and/or activity of one or more immunoprotective proteins comprises expressing or increasing expression of a CTLA-4 gene and/or gene product.

34. The method of any one of the preceding claims, wherein expressing or increasing expression and/or activity of one or more immunoprotective proteins comprises expressing or increasing expression of a CD200 gene and/or gene product.

35. The method of any one of the preceding claims, wherein expressing or increasing expression and/or activity of one or more immunoprotective proteins comprises expressing or increasing expression of a chimeric CD24/CD200 gene and/or gene product or a chimeric CD47/CD200 gene and/or gene product.

36. The method of any one of the preceding claims, wherein expressing or increasing expression and/or activity of one or more immunoprotective proteins comprises expressing or increasing expression of an MFG-E8 gene and/or gene product.

37. The method of any one of the preceding claims, wherein expressing or increasing expression and/or activity of one or more immunoprotective proteins comprises expressing or increasing expression of a NCAM gene and/or gene product.

38. The method of any one of the preceding claims, wherein expressing or increasing expression and/or activity of one or more immunoprotective proteins comprises expressing or increasing expression of an α-phagocytic integrin gene and/or gene product.

39. The method of any one of the preceding claims, wherein expressing or increasing expression and/or activity of one or more immunoprotective proteins comprises expressing or increasing expression of an antibody or antibody format molecule which targets an IL-6 surface receptor (anti-IL-6R).

40. The method of any one of the preceding claims, wherein expressing or increasing expression and/or activity of one or more immunoprotective proteins comprises expression of a FasL gene and/or gene product.

41. The method of any one of the preceding claims, wherein expressing or increasing expression and/or activity of one or more immunoprotective proteins does not comprise overexpression of a FasL gene and/or gene product.

42. The method of any one of the preceding claims, wherein the hypoimmunogenic cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

43. The method of any one of claims 1-41, wherein the hypoimmunogenic cell has expression or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

44. The method of any one of claims 1-41, wherein the hypoimmunogenic cel has reduced or ablated expression and/or activity of a gene and/or gene product of HLA-A, HLA-B, HLA-C, HLA-F, CIITA, IL-6, TRAC, TRBC, and one of either HLA-E or HLA-G.

45. The method of any one of claims 1-41, wherein the hypoimmunogenic cell has reduced or ablated expression and/or activity of a gene and/or gene product of HLA-A, HLA-B, HLA-C, HLA-F, CIITA, IL-6, TRAC, TRBC, SerpinB9, and one of either HLA-E or HLA-G.

46. The method of any one of claims 1-41, wherein the hypoimmunogenic cell has reduced or ablated expression and/or activity of a gene and/or gene product of HLA-A, HLA-B, HLA-C, HLA-F, CIITA, IL-6, TRAC, TRBC, SerpinB9, one of either HLA-E or HLA-G, and one or more of IL-4, IL-10, and IL-16.

47. The method of any one of claims 1-41, wherein the hypoimmunogenic cell expresses or has increased expression of α-phagocytic integrin, CCL2, H2-M3, FasL, MFG-E8, and PD-L1 and/or CTLA-4, wherein the hypoimmunogenic cell does not overexpress FasL, and wherein the hypoimmunogenic cell is not activated with the expression of PD-L1; and
expresses or has increased expression of one of either CD24, CD47, CD200, chimeric CD24/CD47, chimeric CD24/CD200, and chimeric CD47/CD200, or two of either CD24, CD47, and CD200.

48. The method of any one of claims 1-41, wherein the hypoimmunogenic cell expresses or has increased expression of α-phagocytic integrin, CCL2, H2-M3, FasL, MFG-E8, SerpinB9, and PD-L1 and/or CTLA-4, wherein the hypoimmunogenic cell does not overexpress FasL, and wherein the hypoimmunogenic cell is not activated with the expression of PD-L1; and
expresses or has increased expression of one of either CD24, CD47, CD200, chimeric CD24/CD47, chimeric CD24/CD200, and chimeric CD47/CD200, or two of either CD24, CD47, and CD200.

49. The method of any one of claims 1-41, wherein the hypoimmunogenic cell expresses or has increased expression of a CD200 gene and/or gene product and does not express and/or substantially lacks either a CD24 or a CD47 gene and/or gene product.

50. The method of any one of claims 1-41, wherein the hypoimmunogenic cell has no expression and/or activity of a SerpinB9 gene and/or gene product and a CD200 gene and/or gene product.

51. The method of any one of claims 26-40, wherein expressing or increasing expression of the one or more immunoprotective proteins is by introduction of an exogenous genetic element.

52. The method of claim 51, wherein the introduction of the exogenous genetic element is by stable integration into the cell genome.

53. The method of claim 52, wherein the stable integration is by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, or a combination thereof.

54. The method of claim 53, wherein the stable integration is by a viral vector.

55. The method of claim 51, wherein the introduction of the exogenous genetic element is by transient transfection.

56. The method of any one of claims 26-40, wherein expressing or increasing expression of the one or more immunoprotective proteins is by an exogenous promoter and/or enhancer, and/or an endogenous promoter and/or enhancer, or a combination thereof.

57. The method of any one of claims 26-40, wherein expressing or increasing expression of the one or more immunoprotective proteins is under the control of a constitutively active promoter.

58. The method of any one of claims 26-40, wherein expressing or increasing expression of the one or more immunoprotective proteins is at the DNA level by one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

59. The method of any one of the preceding claims, wherein expressing or increasing expression of the one or more immunoprotective proteins is at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

60. The method of any one of claims 26-40, wherein expressing or overexpressing the one or more immunoprotective proteins is by one or more of a small regulatory RNA, miRNA, IRES element, transcription factor, or a combination thereof.

61. The method of any one of the preceding claims, wherein the hypoimmunogenic cell is allogenic.

62. The method of any one of the preceding claims, wherein the hypoimmunogenic cell does not cause an immune reaction in patients to which it or a biological composition derived therefrom is administered.

63. The method of any one of the preceding claims, wherein the hypoimmunogenic cell comprises one or more targeting agents.

64. The method of claim 63, wherein the one or more targeting agents comprises a chimeric antigen receptor (CAR), an immune-incompetent HLA complex, and/or a T cell receptor (TCR).

65. The method of claim 64, wherein the CAR is bispecific.

66. The method of claim 64, wherein the CAR lacks an intracellular portion.

67. The method of claim 64, wherein the CAR comprises a targeting agent, a transmembrane domain, and an intracellular domain, which comprises a costimulatory domain and/or a signaling domain.

68. The method of claim 67, wherein the transmembrane domain is derived from CD28, CD3ζ, CD4, CD8α, or ICOS, or a fragment thereof.

69. The method of claim 67, wherein the intracellular domain comprises an intracellular signaling domain of a CD3ζ-chain and/or one or more co-stimulatory molecules, optionally selected from CD28, 4-1BB, ICOS, CD27, and OX40.

70. The method of claim 63, wherein the one or more targeting agents comprises an antibody or antibody format.

71. The method of claim 70, wherein the antibody or antibody format is selected from one or more of a monoclonal antibody, polyclonal antibody, antibody fragment, Fab, Fab′, Fab′-SH, F(ab′)2, Fv, single chain Fv (scFv), VERR, V_{NAR}, V_{H}H, afflilin, diabody, nanobody, linear antibody, bispecific antibody, multi-specific antibody, chimeric antibody, humanized antibody, human antibody, and fusion protein comprising the antigen-binding portion of an antibody.

72. The method of claim 71, wherein the antibody format is a scFv.

73. The method of claim 63, wherein the one or more targeting agents comprises a viral epitope recognition receptor (VERR) or viral ligand.

74. The method of claim 63, wherein the one or more targeting agents comprises a ligand for a receptor.

75. The method of claim 63, wherein the one or more targeting agents comprises a receptor for a ligand.

76. The method of claims 63-75, wherein the one or more targeting agents is operably linked to a regulatable expression element.

77. A hypoimmunogenic cell produced by the method of any one of claims 1-76.

78. A pharmaceutical composition comprising a hypoimmunogenic cell of claim 77 and one or more excipients.

79. A hypoimmunogenic cell comprising:
(a) one or more membrane-embedded targeting agents targeted against one or more cellular biomarkers;
(b) expression or increased expression of one or more immunoprotective proteins selected from:
(i) α-phagocytic integrin, CCL2, H2-M3, MFG-E8, and FasL, wherein FasL is not overexpressed;
(ii) PD-L1 and/or CTLA-4, wherein the hypoimmunogenic cell is not activated with the expression of PD-L1; and
(iii) one of either CD24, CD47, CD200, chimeric CD24/CD47, chimeric CD24/CD200, and chimeric CD47/CD200, or two of either CD24, CD47, and CD200; and
(c) substantially lacking expression and/or activity of one or more immunogenic proteins selected from:
(i) HLA-A, HLA-B, HLA-C, HLA-F, CIITA, PD-1, IL-6, T cell alpha chain (TRAC), and T cell beta chain (TRBC); and
(ii) HLA-E or HLA-G.

80. A hypoimmunogenic cell comprising:
(a) one or more membrane-embedded targeting agents targeted against one or more cellular biomarkers;
(b) expression or increased expression of one or more immunoprotective proteins selected from:
(i) α-phagocytic integrin, CCL2, H2-M3, MFG-E8, and FasL, wherein FasL is not overexpressed;
(ii) PD-L1 and/or CTLA-4, wherein the hypoimmunogenic cell is not activated with the expression of PD-L1; and
(iii) either CD24 and CD47, or a chimeric CD24/CD47; and
(c) substantially lacking expression and/or activity of one or more immunogenic proteins selected from:
(i) HLA-A, HLA-B, HLA-C, HLA-F, CIITA, PD-1, SerpinB9, IL-6, T cell alpha chain (TRAC), and/or T cell beta chain (TRBC); and
(ii) HLA-E or HLA-G.

81. The hypoimmunogenic cell of claim 79 or 80, wherein the hypoimmunogenic cell is substantially lacking expression and/or activity of one or more of IL-4, IL-10, and/or IL-16.

82. The hypoimmunogenic cell of any one of claims 79-81, further comprising expression or increased expression of an antibody or antibody format molecule which targets an IL-6 surface receptor (anti-IL-6R).

83. The hypoimmunogenic cell of any one of claims 79-82, further comprising expression or increased expression of NCAM.

84. The hypoimmunogenic cell of any one of claims 79-83, wherein the one or more targeting agents comprises a chimeric antigen receptor (CAR).

85. The hypoimmunogenic cell of any one of claims 79-84, wherein the one or more targeting agents are an antibody or antibody format.

86. The hypoimmunogenic cell of claim 85, wherein the antibody or antibody format is selected from one or more of a monoclonal antibody, polyclonal antibody, antibody fragment, Fab, Fab′, Fab′-SH, F(ab′)2, Fv, single chain Fv (scFv), V_{NAR}, V_{H}H, afflilin, diabody, nanobody, linear antibody, bispecific antibody, multi-specific antibody, chimeric antibody, humanized antibody, human antibody, and fusion protein comprising the antigen-binding portion of an antibody.

87. The hypoimmunogenic cell of any one of claims 79-83, wherein the one or more targeting agents is a viral epitope recognition receptor (VERR) or viral ligand.

88. The hypoimmunogenic cell of any one of claims 79-83, wherein the one or more targeting agents is a ligand for a receptor or a receptor for a ligand.

89. The hypoimmunogenic cell of any one of claims 79-88, wherein the hypoimmunogenic cell is allogenic.

90. The hypoimmunogenic cell of any one of claims 79-89, wherein the hypoimmunogenic cell does not cause an immune reaction in patients to which it or a biological composition derived therefrom is administered.

91. A pharmaceutical composition comprising a hypoimmunogenic cell of any one of claims 79-90 and one or more excipients.
